(19) United States
(12) Patent Application Publication
(10) Pub. No.: US 2007/0009484 A1
(43) Pub. Date: Jan. 11, 2007

(54) Compositions and methods involving MDA-7 for the treatment of cancer

(75) Inventors: Kelly K. Hunt, Fresno, TX (US); Young-Jin Suh, Seoul (KR); Stephen G. Swisher, Fresno, TX (US); Abujiang Pataer, Missouri City, TX (US); Rajagopal Ramesh, Sugar Land, TX (US); Manish Shanker, Baytown, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 11/349,727

(22) Filed: Feb. 8, 2006

Related U.S. Application Data
(60) Provisional application No. 60/650,807, filed on Feb. 8, 2005.
(60) Provisional application No. 60/661,679, filed on Mar. 14, 2005.
(60) Provisional application No. 60/676,096, filed on Apr. 29, 2005.
(60) Provisional application No. 60/749,372, filed on Dec. 12, 2005.

(51) Int. Cl. A61K 48/00; A61K 31/415; A61K 31/365

(52) U.S. Cl. 424/93.2; 514/44; 514/406; 514/471; 424/450

(57) ABSTRACT

The present invention concerns methods and compositions involving MDA-7 protein or an MDA-7-encoding nucleic acid in combination with either 1) a COX-2 selective inhibitor, such as celecoxib, 2) an Hsp90 inhibitor, such as geldanamycin, or a geldanamycin derivative or analog, 3) a vitamin E compound, for the treatment of cancer, 4) a TNF, such as TNF-alpha, 5) a VEGF inhibitor, or 6) an inhibitor of IL-10. In certain examples, a treatment for breast cancer is provided. In other examples a treatment for lung cancer is provided. Such examples involve, in some cases, an adenovirus vector that expresses MDA-7 protein.

The present application is related to U.S. Provisional Patent Application 60/650,807, filed on Feb. 8, 2005, U.S. Provisional Patent Application 60/661,679, filed on Mar. 14, 2005, U.S. Provisional Patent Application 60/676,096, filed Apr. 29, 2005, and U.S. Provisional Patent Application 60/749,372, filed Dec. 12, 2005, each of which are hereby incorporated by reference in their entirety.

The government may own rights in the present invention pursuant to grant numbers CA16672, CA78778, and CA102716 from the National Institutes of Health

### BACKGROUND OF THE INVENTION

A. Field of the Invention

The present invention relates generally to the fields of molecular biology and oncology. More particularly, it concerns methods and compositions for treating cancer involving a tumor suppressor, such as MDA-7, and one or more COX-2 inhibitors. This combination of treatment is more effective than each component alone and is greater than their predicted additive effects. In other certain embodiments, the invention concerns methods and compositions for treating cancer with a tumor suppressor such as MDA-7 and an Hsp90 inhibitor, such as geldanamycin and its analogs and derivatives. In further embodiments, the invention relates to methods and compositions for treating cancer involving MDA-7 and a vitamin E compound. The present invention also concerns methods and compositions for treating cancer involving a tumor suppressor, such as MDA-7, and a tumor necrosis factor (TNF). In further embodiments, the present invention concerns methods and compositions for treating cancer involving MDA-7 and a VEGF inhibitor. The present invention also pertains to methods and compositions for treating cancer involving MDA-7 and an IL-10 inhibitor.

B. Description of Related Art

1. MDA-7

Melanoma differentiation-associated gene 7 (mda-7) encodes a 24 kDa protein and is a recently described tumor suppressor gene that induces cell death and apoptosis selectively in cancer cells, while sparing normal cells (Mhashilkar et al., 2001; Mhashilkar et al., 2003; Pataer et al., 2002).

Adenoviral overexpression of MDA-7 leads to tumor selective growth suppression and apoptosis induction in various tumor types including colorectal (Sarkar et al., 2002), breast (Mhashilkar et al., 2003), prostate (Mhashilkar et al., 2001), and lung carcinoma (Chada et al., 2004).

It was recently shown that the combination of adenoviral mediated delivery of mda-7 (Ad-mda7) and trastuzumab increased the anti-tumor activity in HER-2/neu (c-erbB2)-overexpressing breast cancer cells by decreasing phosphorylation of Akt and β-catenin (McKenzie et al., 2004).

It was also demonstrated that adenoviral-mediated overexpression of mda-7 leads to the rapid induction of PKR with subsequent phosphorylation of eIF-2alpha, other PKR target substrates and apoptosis induction in human lung cancer cells (Pataer et al., 2002).

PKR is an interferon induced and double-stranded RNA activated protein kinase. Although best characterized for its function in mediating the antiviral and antiproliferative effects of interferon (IFN), PKR is also implicated in transcriptional regulation, cell differentiation, signal transduction and tumor suppression (Taylor et al., 1999). It is clear that PKR is involved in the regulation of apoptosis, cell-proliferation, signal transduction, and differentiation (Williams, 2001; Barber, 2001; Jagus et al., 1999). It also shown that PKR is regulated by the heat shock protein 90 (Hsp90) molecular chaperone complex (Donze et al., 2001). Hsp90 and its co-chaperone p23 bind to PKR through its N-terminal double-stranded (ds) RNA binding region as well as through its kinase domain (Donze et al, 2001). Both dsRNA and geldanamycin (hereafter referred to as GA) induce the rapid dissociation of Hsp90 and p23 from mature PKR; activate PKR both in vivo and in vitro (Donze et al., 2001). Hsp90 is a chaperone required for the refolding of proteins in cells exposed to environmental stress and for the conformational maturation of several key regulatory proteins (Maloney and Workman, 2002).

2. NSAIDS

There is an increasing body of experimental and epidemiological data suggesting that aspirin, and some other non-steroidal anti-inflammatory drugs (NSAID), exert a chemopreventive action on colorectal cancers and maybe also on stomach, esophagus (Thun et al., 1991) and even bladder (Earnest et al., 1992) cancers. Aspirin, ibuprofen, piroxicam (Reddy et al., 1990; Singh et al., 1994), indomethacin (Narisawa, 1981), and sulindac (Piazza et al., 1997; Rao et al., 1995), effectively inhibit colon carcinogenesis in the AOM-treated rat model and flurbiprofen has demonstrated anti-tumor effects in the APC(Min)+mouse model (Wechter et al., 1997). NSAIDs also inhibit the development of tumors harboring an activated Ki-ras (Singh and Reddy, 1995).

NSAIDs appear to inhibit carcinogenesis via the induction of apoptosis in tumor cells (Bedi et al., 1995; Lupulescu, 1996; Piazza et al., 1995; Piazza et al., 1997b). A number of studies suggest that the chemopreventive properties of the NSAIDs, including the induction of apoptosis, is a function of their ability to inhibit prostaglandin synthesis (reviewed in DuBois et al., 1996; Lupulescu, 1996; Vane and Botting, 1997). It is hypothesized that this may be effected by the inhibition of cyclooxygenase (COX) activity, which suppresses the synthesis of proinflammatory prostaglandins (Hinz et al., 1999). Epidemiological and laboratory studies suggest that colon carcinogenesis is, at least in part, mediated through modulation of prostaglandin production by COX isozymes (COX-1 and -2) (Kawamori et al., 1998). Recent studies, however, indicate that NSAIDs may inhibit carcinogenesis through both prostaglandin-dependent and -independent mechanisms (Alberts et al., 1995; Piazza et al., 1997a; Thompson et al., 1995; Hanif, 1996). Sulindac sulfone, a metabolite of the NSAID sulindac, lacks COX-inhibitory activity yet induces apoptosis in tumor cells (Piazza et al., 1995; Piazza et al., 1997b) and inhibits tumor development in several rodent models of carcinogenesis (Thompson et al., 1995; Piazza et al., 1995, 1997a). It is hypothesized that a potential mechanism of sulindac activity may be the direct or indirect inhibition of tyrosine kinase (Winde et al., 1998), rather than the COX inhibition of the other NSAID agents.

Several NSAIDs have been examined for their effects in human clinical trials. A phase IIa trial (one month) of ibuprofen was completed and even at the dose of 300 mg/day, a significant decrease in prostoglandin E₂ (PGE₂) levels in flat mucosa was seen. A dose of 300 mg of ibuprofen is very low (therapeutic doses range from 1200-3000 mg/day or more), and toxicity is unlikely to be seen, even over the long-term. However, in animal chemoprevention models, ibuprofen is less effective than other NSAIDs. Studies have suggested a beneficial effect of the NSAID, aspirin, on colon cancer incidence, with effects being evident only at a weekly total dose of 1000 mg or greater (Giovannucci et al., 1994). However, three large cohort studies have produced conflicting reports on the beneficial effect of aspirin (Gann et al., 1993; Giovannucci et al., 1996; Greenberg et al., 1993). One group of investigators has recently shown that PGE₂, can be decreased at a dose between 80 and 160 mg/day. In contrast, another group of investigators have shown no such effect on colon mucosal prostaglandins at these low doses of aspirin, although substantial education of prostaglandins in upper gastrointestinal mucosa was demonstrated. The results of these studies indicate that a dose of aspirin of 80 mg is at the threshold of effect of this agent on colon mucosa. Thus, aspirin is not generally recommended for the primary chemoprevention of colorectal cancer in the general population due to questions regarding its efficacy coupled with significant risks of serious cerebrovascular and gastrointestinal adverse effects associated with long-term aspirin use (Singh, 1998).

The NSAID piroxicam is the most effective chemoprevention agent in animal models (Pollard and Luckert, 1989; Reddy et al., 1987; Ritland and Gendler, 1999), although it demonstrated side effects in a recent IIb trial. A large meta-analysis of the side effects of the NSAIDs also indicates that piroxicam has more side effects than other NSAIDs (Lanza et al., 1995). In addition, it has been suggested in at least one study that while tumors of the upper gastrointestinal tract are susceptible to pyroxicam treatment, those of the duodenum and colon are relatively resistant (Ritland and Gendler, 1999). Sulindac has been shown to produce regression of adenomas in Familial Adenomatous Polyposis (FAP) patients (Muscat et al, 1994), although at least one study in sporadic adenomas has shown no such effect (Ladenheim et al., 1995).

Thanks to the rapid pace of development of novel molecular technologies and the completion of the human genome project, new therapeutic targets are being explored against cancer signaling pathways. A recent rationally designed drug is celecoxib—a selective cyclooxygenase 2 (COX-2) inhibitor, which has shown activity as an anti-inflammatory agent (Garner et al., 2002) and in the chemoprevention setting (Kismet et al., 2004). Cyclooxygenase 2 (previously termed prostaglandin endoperoxide H synthase) is one of the two isoforms of cyclooxygenase. Different from the constitutively expressed cyclooxygenase 1, COX-2 is an inducible enzyme and shows highly increased expression in many tumor types, including colon, breast, lung and gastric cancers, suggesting a causal role for COX-2 in oncogenesis (Koehne and Dubois, 2004; Howe et al., 2001). Recently, celecoxib has been shown to promote growth arrest and induce apoptosis in various cancers, including breast cancer, by down-regulating the prosurvival signaling kinase, protein kinase B (PKB)/Akt (Basu et al., 2004; Kulp et al., 2004; El-Rayes et al., 2004; Leng et al., 2003).

HER-2/neu overexpression is a hallmark of aggressive, invasive breast cancers (Ross et al., 2003). Recent studies have also suggested that COX-2 overexpression may be a prognostic marker for aggressive breast cancers and appears to correlate with poor survival (Denkert et al., 2004). High levels of COX-2 are found in HER-2/neu positive tumors and it has been proposed that a positive feedback loop exists between these markers (Benoit et al., 2004). COX-2 expression increases transcription of the HER-2/neu gene and inhibition of COX-2 results in decreased HER-2/neu levels. Increased expression of HER-2/neu results in constitutive signaling via phosphatidylinositol 3-kinases (PI3K) to Akt/protein kinase B (PKB) (Le et al., 2005). Activation of these serine/threonine kinases results in cell proliferation and survival signaling (Craven et al., 2003). It was previously shown that Ad-mda7 negatively regulates Akt survival pathways in breast cancer cells (McKenzie et al., 2004).

3. Hsp90 Inhibitors

Geldenymicin (GA) and 17-allyl-amino-geldanamycin (17AAG) can bind specifically to the ATP-binding site in the NH₂-terminal portion of Hsp90, and inhibit its function. Inhibition of Hsp90 function leads to the degradation of its client proteins, including steroid receptors, HER2, and the Raf, PDK1, Akt and cdk4 kinases (Sausville et al., 2003). In contrast, previous study has shown that GA can induce the rapid dissociation of Hsp90 and p23 from mature PKR, activate PKR both in vivo and in vitro (Donze et al., 2001). Geldanamycin (GA) is a naturally occurring anasamycin antibiotic that has significant anticancer properties. 17-allylamino, 17-demethoxygeldanamycin (17AAG), a geldanamycin derivative, showed good activity and cancer selectivity in preclinical models and has now progressed to Phase I, Phase II clinical trial in cancer patients with encouraging initial results (Kamal et al., 2003). Combination treatment with 17AAG and acute irradiation produces supra-additive growth suppression in human prostate carcinomas (Enmon et al., 2003). Combination therapy using low levels of 17AAG was found to enhance the effects of Taxol and doxorubicin on HER2-overexpressing breast cancer cell lines (Solit et al., 2003). Previous studies implicated 17AAG disruption of the PI3K/AKT pathway in breast cancer cells, and down-regulation of AKT has been associated with enhanced susceptibility of colon tumor cells to butyrate-induced apoptosis (Rahmani et al., 2003).

4. Vitamin E Compounds

Studies have been published describing anti-tumor activity of vitamin E succinate (VES) (Prasad et al., 1982). Recent studies have indicated that VES administered intraperitoneally has anti-tumor activity in animal xenograft and allograft models (Malafa et al., 2000; Malafa et al., 2002). Investigations have shown that VES induces concentration- and time-dependent inhibition of cancer cell growth by blocking DNA synthesis, inducing cellular differentiation and inducing apoptosis (Kline et al., 1998; Kline et al., 2001; Neuzil et al., 2001; You et al, 2001; You et al., 2002; Yu et al., 2001).

5. Cancer

Almost half of all men and more than a third of all women will be afflicted with cancer over their lifetime. Over a million people are diagnosed with it every year. While cancer death rates are generally on a decline, many people continue to die each year from some form of cancer. Lung, colon/rectal, prostate, and breast are the cancers that cause the most deaths.

Among the various cancers threatening women's health, breast cancer is the second most frequent cause of cancer-related deaths among American women. Approximately 15% of all cancer deaths reported in women area due to breast cancer and it's incidence lags only slightly behind lung cancer (Jel et al., 2002). Furthermore, breast cancer is one of the leading causes of cancer mortality in most of the developed and developing countries throughout the world (Pisani et al., 1999). Although considerable progress has been achieved through the development of new drugs and treatment modalities, there still remains an urgent need to improve therapeutic outcomes while reducing treatment-related toxicities (Peto et al., 2000). This remains true for other cancers as well.

The present invention addresses the need for new and improved treatments for cancer.

### SUMMARY OF THE INVENTION

The present invention generally provides methods and compositions for treating cancer using MDA-7 in combination with one or more additional therapeutic agents. The additional therapeutic agents include a COX-2 inhibitor, an Hsp90 inhibitor, a vitamin E compound, a TNF, a VEGF inhibitor, or an IL-10 inhibitor.

In some embodiments, the present invention provides methods and compositions for treating cancer using the combination of MDA-7 and a COX-2 inhibitor. The present invention also provides methods and compositions for treating cancer using the combination of MDA-7 and an Hsp90 inhibitor. In additional embodiments, the present invention concerns methods and compositions for treating cancer using a combination of MDA-7 and a vitamin E compound. In still further embodiments, the present invention concerns methods and compositions for treating cancer using a combination of MDA-7 and a tumor necrosis factor (TNF). The present invention also pertains to methods and compositions for treating cancer using a combination of MDA-7 and a vascular endothelial growth factor (VEGF) inhibitor. In still further embodiments, the present invention concerns methods and compositions for treating cancer using MDA-7 and an IL-10 inhibitor.

Methods of the invention specifically include methods for treating cancer in a patient comprising providing to the patient MDA-7 in combination with i) a COX-2 inhibitor, ii) an Hsp90 inhibitor; iii) a vitamin E compound; iv) a TNF; v) a VEGF inhibitor; vi) an IL-10 inhibitor; or, vii) a combination of one or more of i), ii), iii), iv), v), and vi). When these compounds i), ii), iii), iv), v), or vi) are used in the context of MDA-7, they may collectively be referred to as “MDA-7 conjunctive agent” and any combination therapy with MDA-7 and i), ii), iii), iv), v), or vi) may be referred to as “MDA-7 conjunctive therapy.” The amount provided may be considered an “effective amount” in certain embodiments.

In certain embodiments, the MDA-7 is provided to the cells by administering to the cells an expression construct encoding MDA-7, which then expresses MDA-7 in the cell. In particular embodiments the expression construct is a viral vector. In further embodiments, the viral vector is an adenovirus vector containing a nucleic acid sequence encoding MDA-7.

In some embodiments, the MDA-7 nucleic acid composition includes one or more lipids. For example, the composition may include DOTAP and cholesterol, or a derivative thereof. In some embodiments, an anti-inflammatory agent is administered before, during, or after administration of the MDA-7.

The MDA-7 may be provided to the patient by administering to the patient a composition that includes purified MDA-7 protein. In certain embodiments, the method includes subjecting the patient to radiotherapy, chemotherapy, and/or surgical resection of premalignant or malignant lesion.

In certain embodiments, it also concerns methods and compositions for treating breast cancer cells by providing MDA-7 and an MDA-7 conjunctive agent to the cells. In a particular embodiment, the MDA-7 conjunctive agent is a COX-2 inhibitor to the cells.

Some embodiments concern methods for radiosensitizing cancer cells in a patient comprising providing MDA-7 and an MDA-7 conjunctive agent to the cells. The term “radiosensitizing” means that the cells are rendered more susceptible to the damaging effects of radiation.

It will be understood that “an effective amount” means that the patient is provided with both 1) MDA-7 and 2) any of i) a COX-2 inhibitor, ii) an Hsp90 inhibitor, iii) a vitamin E compound, iv) a TNF, v) a VEGF inhibitor, or vi) an IL-10 inhibitor or other agent used with MDA-7 therapy in an amount or amounts that leads to a therapeutic benefit. It will be understood that the patient is given an amount of MDA-7 and an amount of i) a COX-2 inhibitor, ii) an Hsp90 inhibitor, iii) a vitamin E compound, iv) a TNF, v) a VEGF inhibitor, or vi) an IL-10 inhibitor, both of which amounts are believed to contribute to the therapeutic benefit. In embodiments, in which more than two different compounds are provided with MDA-7, such as a combination of vitamin E compounds or the combination of a vitamin E compound and a COX-2 inhibitor, it will be understood that the term “effective amount” means that the patient is provided with an amount that provides a therapeutic benefit as a result of the amount of the combination of substances that is provided to the patient.

In certain embodiments, the composition contains an MDA-7 polypeptide and another anti-cancer agent, such as a COX-2 inhibitor, an Hsp90 inhibitor, vitamin E compound, or other MDA-7 conjunctive agent such as a VEGF inhibitor, TNF, or an IL-10 inhibitor. Accordingly, in some methods of the invention, the patient is administered a composition comprising purified MDA-7 protein. The term “purified” means that MDA-7 protein was previously isolated away from other proteins and that the protein is at least about 95% pure prior to being formulated in the composition. In certain embodiments, the purified MDA-7 protein is about or is at least about 95, 96, 97, 98, 99, 99.1, 99.2, 99.3, 99.4, 99.5% pure or more, or any range derivable therein. Moreover, it is contemplated that purified MDA-7 protein is active, meaning it is capable of inducing apoptosis. The purified MDA-7 protein can also be qualified in terms of activity such that it is about, at least about, or at most about 50, 55, 60, 65, 70, 75, 80, 85, 90, 95% or more (or any range derivable therein) as active (measured by apoptotic activity) as an equivalent amount of MDA-7 that is not purified (such as prepared by recombinant means).

In other embodiments, it contains a compound that can lead to an MDA-7 polypeptide a COX-2 inhibitor, an Hsp90 inhibitor, vitamin E, VEGF inhibitor, TNF polypeptide, or IL-10 inhibitor in the patient or in cells of the patient.

A patient is any animal with cancer that undergoes treatment. In many embodiments of the invention, a patient is a mammal, specifically a human.

The cancer can be any type of cancer. For example, the cancer may be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the cancer involves epithelial cancer cells. In specific embodiments, the cancer is breast cancer, lung cancer, or prostate cancer.

The subject can be a subject who is known or suspected of being free of a particular disease or health-related condition at the time the relevant preventive agent is administered. The subject, for example, can be a subject with no known disease or health-related condition (i.e., a healthy subject). In some embodiments, the subject is a subject at risk of developing a particular disease or health-related condition. For example, the subject may have a history of cancer that has been treated in the past, who is at risk of developing a recurrence of the cancer. The subject may be a subject at risk of developing a recurrent cancer because of a genetic predisposition or as a result of past chemotherapy. Alternatively, the subject may be a subject with a history of successfully treated cancer who is currently disease-free, but who is at risk of developing a second primary tumor. For example, the risk may be the result of past radiation therapy or chemotherapy that was applied as treatment of a first primary tumor. In some embodiments, the subject may be a subject with a first disease or health-related condition, who is at risk of development of a second disease or health-related condition.

“Treatment” and “treating” refer to administration or application of an agent, drug, or remedy to a subject or performance of a procedure or modality on a subject for the purpose of obtaining a therapeutic benefit of a disease or health-related condition.

A “disease” or “health-related condition” can be any pathological condition of a body part, an organ, or a system resulting from any cause, such as infection, genetic defect, and/or environmental stress. The cause may or may not be known. Examples of such conditions include, but are not limited to, premalignant states, dysplasias, cancer, and other hyperproliferative diseases. The cancer, for example, may be a recurrent cancer or a cancer that is known or suspected to be resistant to conventional therapeutic regimens and standard therapies.

The term “therapeutic benefit” used throughout this application refers to anything that promotes or enhances the well-being of the subject with respect to the medical treatment of his condition, which includes, but is not limited to, treatment of pre-cancer, dysplasia, cancer, and other hyperproliferative diseases. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell or tumor cell proliferation rate, decrease or delay in progression of neoplastic development from a premalignant condition, and a decrease in pain to the subject that can be attributed to the subject's condition.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. In certain embodiments of the present invention, the methods involving delivery of MDA-7 protein or a nucleic acid encoding the protein to prevent a disease or health-related condition in a subject. An amount of a pharmaceutical composition that is suitable to prevent a disease or condition is an amount that is known or suspected of blocking the onset of the disease or health-related condition. The invention contemplates that MDA-7 may be provided to a subject in addition to at least one other agent, such as a COX-2 inhibitor or any other MDA-7 conjunctive agent.

In additional embodiments of the invention, methods include identifying a patient in need of treatment. A patient may be identified, for example, based on taking a patient history, having one or more tests done to determine that the patient has cancer or a tumor, operating on the patient or taking a biopsy.

Accordingly, in some embodiments, MDA-7 is provided to the patient by administering to the patient a composition comprising a nucleic acid having a sequence encoding MDA-7 polypeptide, wherein the MDA-7 polypeptide is expressed in the patient. It is contemplated that the MDA-7 encoding nucleic acid sequence is under the control of a promoter capable of providing expression in the patient. The promoter can be constitutive, tissue-specific, repressible, or inducible. In certain embodiments, the promoter is the CMV IE promoter. In additional embodiments, an enhancer is included. A vector, including an expression construct, can be employed in methods of the invention to provide MDA-7 to a patient. In certain embodiments, the vector is a viral vector. If a viral vector is used, in some embodiments the vector is formulated with protamine. In certain embodiments, a vector is formulated with one or more lipids. In some embodiments, a lipid formulation is a DOTAP:cholesterol (or derivative thereof) (DOTAP:chol) formulation.

It is contemplated that compositions administered to a patient may be in a pharmaceutically acceptable formulation.

Viral vectors that can be used are adenovirus, adeno-associated virus, herpesvirus, lentivirus, retrovirus, and vaccinia virus. In specific embodiments, the vector is an adenovirus vector, which can be replication-deficient. In this case, it is contemplated that about 10⁹ to about 10¹³ viral particles (cp) or plaque forming units (pfu) are administered to the patient either per administration (patient/administration) or per day (average daily dose). Such doses include about, at least about, or at most about 10⁹, 10¹⁰, 10¹¹, 10¹², or 10¹³ vp or pfu (or any range derivable therein), which may be the amount given per administration or per day or per treatment cycle.

In fact, embodiments of the invention set forth that the combination of MDA-7 and a COX-2 inhibitor provides a synergistic therapeutic effect with respect to promoting apoptosis in cancer cells. Embodiments of the invention also set forth that the combination of MDA-7 and TNF-alpha provides a synergistic therapeutic effect with respect to inhibiting tumor cell proliferation. In other embodiments of the invention, methods concern the combination of MDA-7 and an Hsp90 inhibitor, which provides a synergistic therapeutic effect with respect to promoting apoptosis in cancer cells. Additional embodiments of the invention set forth that the combination of MDA-7 and one or more vitamin E compounds provides a synergistic therapeutic effect with respect to promoting inhibition of cancer cells. Even further embodiments of the invention set forth that the combination of MDA-7 and a VEGF inhibitor provides a synergistic therapeutic effect with respect to tumor growth inhibition. Embodiments of the invention set forth that the combination of MDA-7 and a TNF polypeptide provides a synergistic therapeutic effect with respect cancer therapy. Additionally, in some embodiments, the combination of MDA-7 and an IL-10 inhibitor provides a synergistic therapeutic effect with respect cancer therapy. “Synergistic” indicates that the therapeutic effect is greater than would have been expected based on adding the effects of each agent applied as a monotherapy.

A patient is provided both MDA-7 and a COX-2 inhibitor in methods of the invention. In other methods, a patient is provided with both MDA-7 and an Hsp90 inhibitor. In still further methods, a patient is provided with both MDA-7 and at least one vitamin E compound. In additional embodiments, a patient is provided with MDA-7 and a VEGF inhibitor. In another embodiment, a patient is provided with both MDA-7 and a TNF polypeptide. In other embodiments, a patient is provided with both MDA-7 and an IL-10 inhibitor. The term “provide” is used according to its ordinary and plain meaning: “to supply or furnish for use” (Oxford English Dictionary). It is contemplated that cancer cells of the patient or cells adjacent to cancer cells of the patient are exposed to MDA-7 and the COX-2, Hsp90 inhibitor, vitamin E compound, VEGF inhibitor, and/or TNF in methods of the invention. MDA-7 exerts a bystander effect and consequently, a cell adjacent to a cancer cell may express MDA-7 and provide it to a cancer cell. In embodiments, of the invention a composition is administered to the patient so as to provide MDA-7 and/or a COX-2 inhibitor to the patient. In other embodiments, a composition is administered to the patient so as to provide MDA-7 and/or an Hsp90 inhibitor to the patient. In still further embodiments, a composition is administered to the patient so as to provide MDA-7 and/or one or more vitamin E compounds to the patient. It is specifically contemplated that a esterified form of a vitamin E compound may be included in the composition. Moreover, in some embodiments, a composition is administered to the patient so as to provide MDA-7 and/or a VEGF inhibitor to the patient. In still further embodiments, a composition is administered to the patient so as to provide MDA-7 and/or a TNF polypeptide to the patient. Additionally, a composition is administered to a patient so as to provide MDA-7 and/or an IL-10 inhibitor to the patient.

Compounds and compositions may be administered to a patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, or via a lavage. It is contemplated that a combination of routes of administration may be employed. For instance, MDA-7 may be provided by one route while an MDA-7 conjunctive agent is provided by another route. Alternatively, it is contemplated that one dose of either a) MDA-7 or b) the COX-2, Hsp90 inhibitor, vitamin E compound, VEGF inhibitor, TNF, or IL-10 inhibitor (MDA-7 conjunctive agent) is administered to the patient while another dose is administered to the patient in a different manner.

In certain embodiments, it is contemplated that a compound(s) or composition(s) is directly injected into or to a tumor. Alternatively or additionally, a compound(s) or composition(s) is applied or administered to a residual tumor bed. Furthermore, in specific embodiments, the COX-2 inhibitor is taken orally by the patient or administered intravenously to the patient. In other embodiments, an Hsp90 inhibitor is taken orally or provided to the patient by infusion or injection. In particular embodiments, the vitamin E compound is taken orally or administered intravenously or peritoneally. In certain embodiments, a VEGF inhibitor or an IL-10 inhibitor is administered by infusion, such as intravenously. In particular embodiments, TNF is administered directly into or to a tumor. It is specifically contemplated that any MDA-7 conjunctive agent can be given intratumorally as well.

MDA-7, a COX-2 inhibitor, an Hsp90 inhibitor, a vitamin E compound, VEGF inhibitor, TNF, or an IL-10 inhibitor can be provided to a patient the following number of time or at least the following number of times: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more times as part of a therapy. It is specifically contemplated that a patient is provided with MDA-7 and the COX-2 inhibitor more than once as part of the patient's cancer treatment. It is also specifically contemplated that in other embodiments, a patient is provided with MDA-7 and the Hsp90 inhibitor more than once as part of the patient's cancer treatment. In even further embodiments, a patient is provided with MDA-7 and a vitamin E compound more than once as part of the patient's cancer treatment. Moreover, it is contemplated that there may be a course of therapy prescribed, and that the course may be repeated, if necessary. This applies to therapy with any other MDA-7 conjunctive agent as well.

The MDA-7 may be administered to the patient either prior to, concurrently with, or following administration of the MDA-7 conjunctive agent. One of ordinary skill in the art would be familiar with therapeutic regimens for administration of more than one therapeutic agent. For example, the patient may be provided with MDA-7 within 24 hours of being provided with the MDA-7 conjunctive agent. In some embodiments, the patient is provided with the MDA-7 within 2 hours of being provided with the MDA-7 conjunctive agent. In further embodiments, the patient is provided with MDA-7 prior to being provided with the MDA-7 conjunctive agent. In further embodiments, the patient is provided with the MDA-7 conjunctive agent prior to being provided with MDA-7.

The present invention can be used to induce apoptosis in cells. It is contemplated that this can be employed in methods and compositions for treating cancer. The cancer can be any of the following types of cancer: melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the cancer involves epithelial cancer cells. In specific embodiments, the cancer is breast cancer. In the case of breast cancer, the patient can be HER-2/neu negative or the patient can be HER-2/neu positive. Thus, the treatment can be independent of HER-2/neu status of the patient.

Moreover, the present invention can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like. The progression to cancer or to a more severe form of cancer may be halted, disrupted, or delayed by methods of the invention involving MDA-7 conjunctive therapy as discussed herein.

Moreover, the cancer may involve an unresectable or resectable tumor. In some embodiments, the cancer appears resistant to radiotherapy, chemotherapy, and/or immunotherapy (such as trastuzumab), or to any of the agents discussed herein, but as a monotherapy (compared to an MDA-7 conjunctive therapy). Furthermore, the cancer may involve a metastasized or second tumor, though in some embodiments, it concerns only one or more primary tumors. It is further contemplated that the methods and compositions of the invention can be implemented for inhibiting metastasis of a tumor or preventing the further growth of a tumor, as well as for reducing or eliminating a tumor or cancer.

In specific embodiments, the present invention concerns methods of treating cancer in which a patient with cancer is provided MDA-7 and a COX-2 inhibitor. Other methods of the invention concern treating breast cancer in a patient comprising administering to the patient a i) an adenovirus vector comprising a nucleic acid sequence encoding MDA-7, wherein the nucleic acid sequence is under the control of a promoter capable of being expressed in the patient; and, ii) a COX-2 inhibitor.

It is contemplated that in some embodiments of the invention, a patient is provided with the COX-2 inhibitor by administering a COX-2 inhibitor directly to the patient. In other embodiments, a prodrug of the COX-2 inhibitor is administered to the patient, and once inside the patient's body, it gets converted into the active form of a COX-2 inhibitor. The present invention contemplates that the COX-2 inhibitor is selected from the group consisting of celecoxib, rofecoxib, valdecoxib, lumiracoxib, and etoricoxib. Moreover, more than one COX-2 inhibitor may be employed, such as a combination of 2, 3, or 4 such inhibitors (and/or their relevant prodrugs).

In more particular embodiments, the MDA-7 conjunctive agent is a COX-2 inhibitor. Radiosensitization with MDA-7 and a COX-2 inhibitor occurs by arresting the tumor cells in the radiosensitive G2/M phase of the cell cycle. It is thus contemplated that the amount or amounts of radiation that cancer cells are typically exposed to may be lowered or reduced after being radiosensitized. Alternatively, the number of radiation sessions or the length of sessions may be reduced. In certain embodiments, the reduction in any single amount, in the total amount, in the number of sessions, or in the length of the sessions is by about, at least about, or at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000% or more, or any range derivable therein.

The present invention also concerns methods for treating cancer in which a cancer patient is provided an MDA-7 and an Hsp90 inhibitor. In certain embodiments, methods involve treating cancer by providing i) an adenovirus vector comprising a nucleic acid sequence encoding MDA-7, wherein the nucleic acid sequence is under the control of a promoter capable of being expressed in the patient; and, ii) an Hsp-90 inhibitor. The term “Hsp-90 inhibitor” refers to a substance that specifically and directly inhibits Hsp90 function. In certain embodiments, the Hsp-90 inhibitor binds to the Hsp90 polypeptide.

In certain embodiments, methods involve treating cancer by providing) an adenovirus vector comprising a nucleic acid sequence encoding MDA-7, wherein the nucleic acid sequence is under the control of a promoter capable of being expressed in the patient; and, ii) an Hsp-90 inhibitor.

It is contemplated that in some embodiments of the invention, a patient is provided with the Hsp90 inhibitor by administering an Hsp90 inhibitor directly to the patient. In other embodiments, a prodrug of the Hsp90 inhibitor is administered to the patient, and once inside the patient's body, it gets converted into the active form of an Hsp90 inhibitor. The present invention contemplates that the Hsp90 inhibitor is in some embodiments geldanamycin, or a derivative or analog of geldanamycin. The term “derivative” refers to a substance produced from another substance either directly or by modification or partial substitution. The term “analog” refers to a substance that is structurally similar or shares similar or corresponding attributes with another molecule (e.g., an geldanamycin variant capable of specifically binding Hsp90). Moreover, more than one Hsp90 inhibitor may be employed, such as a combination of 2, 3, or 4 such inhibitors (and/or their relevant prodrugs).

In certain embodiments the invention concerns methods for treating cancer in which a cancer patient is provided MDA-7 and a vitamin E compound. The term “vitamin E compound” refers to natural and synthetic substances that are lipid-soluble, antioxidant compounds in the tocopherol and tocotrienol subfamilies, as well as esterified forms of such substances and conjugated forms. The tocopherol and tocotrienol families each have alpha (α), beta (β), gamma (γ), and delta (δ) vitamers as members. Esterified forms include acetate and succinate forms. In certain embodiments, the vitamin E compound is synthetic, while in others it is the naturally occurring version of a particular vitamer. In particular embodiments, the vitamin E compound is vitamin E succinate (VES), also known as alpha-tocopheryl succinate.

It is contemplated that in some embodiments of the invention, a patient is provided with the vitamin E compound by administering vitamin E directly to the patient. The term “vitamin E” is understood to any of the eight lipid-soluble, antioxidant tocopherol and tocotrienol compounds. In other embodiments, a prodrug of the vitamin E is administered to the patient, and once inside the patient's body, it gets converted into the active form of a vitamin E. In certain embodiments, the prodrug is an esterified form of vitamin E, such as an acetate or succinate form. The present invention contemplates that the vitamin E compound is in some embodiments alpha-tocopherol, or an esterified form of alpha-tocopherol, such as alpha-tocopheryl succinate or alpha-tocopherol acetate. The term “esterified form” refers to a form of the substance with an ester group. In certain other embodiments, an analog of a vitamin E compound is employed in methods and compositions of the invention instead of the vitamin E compound. The term “analog” refers to a substance that is structurally similar or shares similar or corresponding attributes with another molecule (e.g., Trolox C). In further embodiments, a vitamin E conjugate is employed in methods and compositions of the invention. Moreover, more than one vitamin E compound may be employed, such as a combination of 2, 3, or 4 such vitamers and/or esterified forms.

The present invention also concerns methods for treating cancer in a patient that involve providing MDA-7 and a TNF to the patient. The TNF may be any TNF, such as TNF-alpha, TNF-beta, TNF-gamma, TNF-delta, or TNF-epsilon. In particular embodiments of the present invention, the TNF is TNF-alpha. The TNF may include either the full-length amino acid sequence, or a partial length sequence, so long as the partial length sequence is capable of functioning as a TNF. Also included in the definition of TNF are amino acid sequence variants of the full-length and partial length sequence of TNF, so long as these sequence variants are capable of functioning as a TNF.

In addition, the present invention concerns methods for treating cancer in a patient that involve providing MDA-7 and a VEGF inhibitor to the patient. In the context of cancer treatment, anti-angiogenic factors may rely on the inhibition of the interaction between vascular endothelial growth factor (VEGF) and its receptors. Tumor VEGF expression has been clinically correlated with disease progression in a range of malignancies. Such correlation is thought to be attributed to the ability of VEGF to induce tumor angiogenesis by stimulating the chemotaxis and mitogenesis of endothelial cells, as well as increasing endothelial cell-associated protease activity, and elevating integrin expression in microvascular cells to augment extracellular matrix interactions. Two high-affinity receptors for VEGF with associated tyrosine kinase activity have been identified on human vascular endothelium: Flt-1 and KDR. VEGF binding to these receptors is thought to cause dimerization and a subsequent activation of the receptor tyrosine kinase domain. The VEGF inhibitor may be any VEGF inhibitor known to those of ordinary skill in the art. For example, the inhibitor may be a DNA, RNA, an oligonucleotide, a ribozyme, a protein, a polypeptide, a peptide, an antibody, an oligosaccharide, or small molecule. In particular embodiments, the VEGF inhibitor is an antibody, such as an antibody directed against VEGF or a VEGF receptor. In more particular embodiments, the antibody is a monoclonal antibody, such as a monoclonal antibody that specifically binds VEGF or a VEGF receptor. In a more particular embodiment, the VEGF inhibitor is Bevacizumab (Avastin). In some embodiments, the VEGF inhibitor is a small molecule. Examples of such small molecules include small molecule tyrosine kinase inhibitors of a VEGF receptor, In particular embodiments, the VEGF inhibitor is a ribozyme, such as a ribozyme which specifically targets VEGF mRNA or VEGF receptor mRNA. In further particular embodiments, the VEGF inhibitor is a soluble VEGF receptor.

In some embodiments of the present invention, molecules that inhibit VEGF signaling are contemplated. In certain embodiments inhibition of VEGF signaling may be through receptor tyrosine kinase inhibitors. Receptor tyrosine kinase inhibitors that are contemplated include, but are not limited to: ZD4190, ZD6474, and AZD2171 (Astra-Zeneca, Wilmington, Del.), CEP-7055 (Cephalon, Frazer, Pa.), PTK787 (Novartis, Basel, Switzerland) and SU5416 (Sugen, South San Francisco, Calif.).

The present invention also concerns methods of treating cancer in a patient by providing MDA-7 and an IL-10 inhibitor to the patient. The inhibitor of IL-10 may be any IL-10 inhibitor known to those of ordinary skill in the art. For example, the inhibitor may be a DNA, RNA, ribozyme, oligonucleotide, protein, polypeptide, peptide, antibody, or small molecule. In particular embodiments, the IL-10 inhibitor is an antibody, such as an antibody directed against IL-10. In some embodiments, the antibody is a monoclonal antibody.

As set forth above, the MDA-7 may be provided to the patient by administering to the patient a composition that includes a nucleic acid having a sequence encoding MDA-7 polypeptide, wherein the MDA-7 polypeptide is expressed in the patient. In particular embodiments, the composition is a pharmaceutically acceptable composition. Alternatively, the MDA-7 may be provided to the patient by administering to the patient a purified MDA-7 protein composition, as discussed above. In certain particular embodiments, the composition is a pharmaceutically acceptable composition.

In methods of the present invention that pertain to the providing MDA-7 and TNF, the TNF may be provided to the patient by administering to the patient a composition that includes a nucleic acid having a sequence encoding a TNF, wherein the TNF polypeptide is expressed in the patient. In methods of the present invention that pertain to providing MDA-7 and a VEGF inhibitor, the VEGF inhibitor may be provided to the patient by administering to the patient a composition that includes a nucleic acid having a sequence encoding a VEGF inhibitor, wherein the VEGF inhibitor is expressed in the patient, In methods of the present invention that involve administering MDA-7 and an IL-10 inhibitor, the IL-10 inhibitor may be provided to the patient by administering to the patient a composition that includes a nucleic acid having a sequence encoding a VEGF inhibitor.

It is contemplated that a patient is provided with MDA-7 and a COX-2 inhibitor with a single composition in some embodiments. Compositions to be administered to a patient include compositions of the present invention, which are disclosed herein. Furthermore, in some embodiments, the patient is provided with a composition comprising the COX-2 inhibitor and either i) purified MDA-7 protein or ii) a nucleic acid having a sequence encoding MDA-7. Alternatively, instead of the COX-2 inhibitor, the composition may include a COX-2 inhibitor prodrug.

In other methods of the invention, a patient is provided with MDA-7 and an Hsp90 inhibitor with the administration of a single composition in some embodiments. Compositions to be administered to a patient include compositions of the present invention, which are disclosed herein. Furthermore, in some embodiments, the patient is provided with a composition comprising the Hsp90 inhibitor and either i) purified MDA-7 protein or ii) a nucleic acid having a sequence encoding MDA-7. Alternatively, instead of the Hsp90 inhibitor, the composition may include an Hsp90 inhibitor prodrug.

It is contemplated that a patient is provided with MDA-7 and a vitamin E compound with a single composition in some embodiments. Compositions to be administered to a patient include compositions of the present invention, which are disclosed herein. Furthermore, in some embodiments, the patient is provided with a composition comprising the COX-2 inhibitor and either i) purified MDA-7 protein or ii) a nucleic acid having a sequence encoding MDA-7. Alternatively, instead of vitamin E, the composition may include an esterified form of vitamin E, a vitamin E analog, or a vitamin E conjugate.

It is also contemplated that the embodiments discussed above regarding single compositions apply to other MDA-7 conjunctive agents as well, such as VEGF inhibitors, TNF, or IL-10 inhibitors.

In other embodiments, MDA-7 and a COX-2 inhibitor or other MDA-7 conjunctive agent are provided separately to the patient. Similarly, MDA-7 and an Hsp90 inhibitor are provided separately to the patient in certain embodiments. Furthermore, in additional embodiments, MDA-7 and one or more vitamin E compounds is provided separately to the patient. In those cases, it is contemplated that the patient is provided with one agent and the other agent is provided or administered within 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 day and/or 1, 2, 3, 4, 5, 6, 7, 8, 9, 10, 11, 12 weeks, or any range derivable therein. In certain embodiments, it is contemplated that the patient is provided with MDA-7 within 24 hours of being provided with the COX-2 inhibitor, Hsp90 inhibitor, or vitamin E compound or other MDA-7 conjunctive agent. In other embodiments, the patient is provided with the MDA-7 within 2 hours of being provided with the COX-2 inhibitor, Hsp90 inhibitor, vitamin E compound, or other MDA-7 conjunctive agent. In some embodiments, the patient is provided with the MDA-7 prior to being provided with the COX-2 inhibitor, Hsp90 inhibitor, or vitamin E compound or other MDA-7 conjunctive agent, while in others the patient is provided with the agent prior to being provided with the MDA-7. Furthermore, it is contemplated that a patient may take or be administered a n MDA-7 conjunctive agent throughout the course of treatment with MDA-7. For example, it is contemplated that a patient may undergo MDA-7 therapy for a six week period. During that time, the patient may take, for example, a COX-2 inhibitor, Hsp90 inhibitor, or vitamin E compound throughout the six-week period, such as at least on a daily or weekly basis. Therefore, it is contemplated that a patient may take or be provided with a COX-2 inhibitor, Hsp90 inhibitor, vitamin E compound, or other MDA-7 conjunctive agent within 24 hours (or any time period specified above) of being provided MDA-7 and that MDA-7 may be provided more than once. Accordingly, the patient will have taken or be provided with a COX-2 inhibitor, Hsp90 inhibitor, vitamin E compound, VEGF inhibitor, TNF, or IL-10 inhibitor within 24 hours of each time that MDA-7 is provided to the patient (either as a protein or a nucleic acid encoding the protein). It is furthermore contemplated that within any time period specified above, the COX-2 inhibitor, Hsp90 inhibitor, or vitamin E compound or or other MDA-7 conjunctive agent may be taken or be provided multiple times. For example, a patient may take three doses of a COX-2 inhibitor within 24 hours of being provided with MDA-7. Consequently, a patient may take or be provided an MDA-7 conjunctive agent 1, 2, 3, 4, 5, 6, 7, 8, 9, 10, 11, 12, 13, 14, 15, 16, 17, 18, 19, 20 or more individual times, or any range derivable therein, within a specified time period of being provided the MDA-7. Alternatively, the COX-2 inhibitor, Hsp90 inhibitor, vitamin E compound, or other MDA-7 conjunctive agent may be provided systemically during or throughout treatment with MDA-7.

In some embodiments the patient is subjected to radiotherapy after being provided MDA-7 and a COX-2 inhibitor (or other MDA-7 conjunctive agent) each at least once. In further embodiments a patient is subjected to a sub-lethal dose of radiotherapy. The term “sub-lethal dose” refers to an amount of radiation given to a patient in a single session that is less than a lethal amount (i.e., amount that causes cell to die) for cells of the patient exposed to the radiation. It is contemplated that a sub-lethal dose is lower than the dose currently given to a cancer patient with similar characteristics (referring to, e.g., stage of cancer, size of tumor, prognosis, etc.) who are not first provided with radiosensitization treatment. It is contemplated that the radiosensitization treatment may preceed exposure to radiation by about, at least about, or at most about 5, 10, 15, 20, 25, 30, 35, 40, 45, 50, 55, 60 minutes, and/or 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days, or more, or any range derivable therein.

In certain embodiments of the invention, methods also include subjecting the patient to radiotherapy and/or chemotherapy. In other embodiments, the patient is subjected to immunotherapy. In other particular embodiments, methods also involve resecting all or part of a tumor from the patient. It is contemplated that multiple tumors may be removed (whole or part). In each of these cases, MDA-7 and/or a COX-2 inhibitor, Hsp90 inhibitor, a vitamin E compound, a VEGF inhibitor, a TNF, and/or an IL-10 inhibitor can be provided, before, during or after the other cancer therapy. In certain embodiments, MDA-7 and/or an MDA-7 conjunctive agent is provided to the patient after tumor resection, such as by administering a composition with one or multiple agents to at least the resulting tumor bed.

In other embodiments, the patient is subjected to resection of all or part of a tumor from a patient. The MDA-7 and MDA-7 conjunctive agent may be administered before, during, or after resection of all or part of the tumor from the patient. In some embodiments, the MDA-7 and MDA-7 conjunctive agent is provided after resection of all or part of the tumor from the patient. In some embodiments, the patient is provided the MDA-7 and MDA-7 conjunctive agent at least by administering a composition to the resulting tumor bed.

The MDA-7 and MDA-7 conjunctive agent may be administered once, or more than once. In particular embodiments wherein either the MDA-7 or MDA-7 is an adenovirus vector, the patient may be administered the adenovirus vector once or more than once.

Other embodiments of the invention include providing a different tumor suppressor in place of MDA-7 in embodiments of the invention. Other tumor suppressors include, but are not limited to p53, FUS1, C-CAM, FHIT, DCC, Rb, and PTEN. As such, the protein or a nucleic acid encoding the utmor suppressor may be employed as discussed above.

The present invention also concerns pharmaceutical compositions. In some embodiments, there is a pharmaceutical composition that includes a) a COX-2 inhibitor or COX-2 inhibitor prodrug; and b) purified and active MDA-7 protein or a nucleic acid having a sequence encoding MDA-7 polypeptide. It is contemplated that in embodiments involving a MDA-7 encoding nucleic acid, the nucleic acid may be an adenovirus vector. Pharmaceutical compositions may contain one or more of celecoxib, rofecoxib, valdecoxib, lumiracoxib, and etoricoxib. In certain embodiments, it contains celecoxib.

Other pharmaceutical compositions include a) an Hsp90 inhibitor or an Hsp90 inhibitor prodrug; and b) purified and active MDA-7 protein or a nucleic acid having a sequence encoding MDA-7 polypeptide. It is contemplated that in embodiments involving a MDA-7 encoding nucleic acid, the nucleic acid may be an adenovirus vector. In particular embodiments the composition includes GA or 17-GAA. In even further embodiments, pharmaceutical compositions contain geldanamycin or a geldanamycin derivative or analog, or prodrug thereof.

The present invention also concerns pharmaceutical compositions that include a) at least one vitamin E compound; and b) purified and active MDA-7 protein or a nucleic acid having a sequence encoding MDA-7 polypeptide. It is contemplated that in embodiments involving a MDA-7 encoding nucleic acid, the nucleic acid may be an adenovirus vector. In particular embodiments the composition includes VES.

In some embodiments, the TNF is provided to the patient by administering to the patient a composition that includes purified TNF. As set forth above, this may be a full-length TNF amino acid sequence, or a partial length sequence that maintains biological function as a TNF, or a sequence variant of a full or partial length TNF, so long as that sequence maintains some level of biological activity. Similarly, in embodiments that pertain to administration of MDA-7 and a VEGF inhibitor, the VEGF inhibitor, when a protein, may be administered as a purified protein. Similarly, in embodiments that pertain to administration of MDA-7 and an IL-10 inhibitor, the IL-10 inhibitor, when an amino acid sequence, may be provided to the patient in a composition that includes the purified protein.

In particular embodiments, the patient is provided with a composition that includes a purified MDA-7 protein or a nucleic acid having a sequence encoding MDA-7, and a purified TNF amino acid sequence. In a further particular embodiment, the patient is provided with a composition that includes a TNF and either purified MDA-7 protein or a nucleic acid having a sequence encoding MDA-7. In more particular embodiment, the TNF protein is TNF-alpha protein.

In further embodiments, the patient is provided with at least the following: (a) purified MDA-7 protein or a nucleic acid having a sequence encoding MDA-7 protein; and (b) a monoclonal antibody that specifically binds VEGF. In certain particular embodiments, the monoclonal antibody is Bevacizumab.

In still further embodiments, the patient is provided with a composition that includes: (a) purified MDA-7 protein or a nucleic acid having a sequence encoding MDA-7 protein; and (b) an antibody that specifically binds IL-10 or a nucleic acid having a sequence encoding an antibody that specifically binds IL-10. In certain particular embodiments, the monoclonal antibody is Bevacizumab.

The present invention also generally concerns pharmaceutical compositions that include (a) a purified and active TNF or a nucleic acid having a sequence encoding a TNF; and (b) purified and active MDA-7 protein or a nucleic acid having a sequence encoding MDA-7 polypeptide. In particular embodiments, the purified and active TNF is purified and active TNF-alpha. In further particular embodiments, the nucleic acid having a sequence encoding a TNF is a nucleic acid having a sequence encoding a TNF-alpha polypeptide.

In certain embodiments, the pharmaceutical composition includes a nucleic acid having a sequence encoding MDA-7 polypeptide. In particular embodiments, the nucleic acid encoding MDA-7 polypeptide is an adenovirus vector. The pharmaceutical composition may include a nucleic acid having a sequence encoding a TNF polypeptide, such as a TNF-alpha polypeptide. The nucleic acid encoding the TNF polypeptide may be an adenovirus vector. In a particular embodiment, the pharmaceutical composition includes (a) a first adenovirus vector having a nucleic acid sequence encoding MDA-7, wherein the nucleic acid sequenc is operably coupled to a first promoter sequence; and (b) a second adenovirus vector having a sequence encoding a TNF polypeptide, wherein the nucleic acid sequence encoding the TNF polypeptide is operably connected to a second promoter. The TNF polypeptide may, for example, be a TNF-alpha polypeptide.

In some embodiment, the pharmaceutical composition includes an adenovirus vector having a first nucleic acid sequence encoding MDA-7 and a second nucleic acid sequence encoding a TNF polypeptide. In a more particular embodiment, the second nucleic acid sequence encoding a TNF polypeptide is a nucleic acid sequence encoding a TNF-alpha polypeptide. The first nucleic acid sequence and the second nucleic acid sequence may or may not be operably connected to one or more common promoters.

The present invention also pertains to methods of treating or preventing cancer in a patient, that include administering to the patient a pharmaceutically acceptable composition comprising a polynucleotide encoding an MDA-7 protein and a lipid. The cancer can be any of those cancers set forth above. In certain embodiments, the patient is a patient with lung cancer. For example, the lung cancer may be a non-small cell lung, small-cell lung, or a metastatic lung cancer (cancer that has spead outside the confines of the lung). Treatment of the primary lung cancer may be effected, in addition to treatment of a secondary tumor from the lung cancer. In further embodiments, the method is further defined as a method of treating metastatic lung cancer in a subject.

Any lipid suitable for pharmaceutical administration is contemplated by the present invention. In certain embodiments, the composition is further defined as comprising a liposome. Any liposome suitable for pharmaceutical administration is contemplated for inclusion in the methods of the present invention. In certain embodiments, the liposome is a DOTAP:cholesterol nanoparticle. Liposomes and nanoparticles are discussed in greater detail in the specification below. The method/route of administration can be any method known to those of ordinary skill in the art, such as intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, and/or via a lavage.

Other embodiments of the present invention pertain to methods for treating cancer in a patient that include providing MDA-7 and taxotere (docetaxel) to the patient. The MDA-7 may be provided to the patient by any method known to those of ordinary skill in the art. For example, the MDA-7 may be provided to the patient by administering to the patient a composition comprising a nucleic acid having a sequence encoding MDA-7 polypeptide, wherein the MDA-7 polypeptide is expressed in the patient.

In some embodiments, the MDA-7 is provided to the patient by administering to the patient a composition that includes purified MDA-7 protein. The composition may include one or more additional anti-cancer agents, such as other chemotherapeutic agents or an MDA-7 conjunctive agent. Exemplary chemotherapeutic agents are set forth in the specification below. In further embodiments, the patient is being treated with one or more additional anti-cancer therapies. Examples of such therapies include radiation therapy, additional chemotherapy, immunotherapy, other forms of gene therapy, and surgical therapy.

In certain embodiments, the the patient is provided with a composition that includes taxotere and either i) purified MDA-7 protein or ii) a nucleic acid having a sequence encoding MDA-7. The MDA-7 may be provided prior to, during, or after administration of taxotere. In some embodiments, for example, the patient is provided with MDA-7 within 24 hours of being provided with the taxotere. More particularly, the patient may be provided with the MDA-7 within 2 hours of being provided with the taxotere. The patient may be provided with the MDA-7 prior to being provided with the taxotere, or the patient may be provided with taxotere prior to being provided with MDA-7. In certain embodiments, the patient is provided with taxotere by administering to the patient taxotere.

The cancer can be any of those cancers discussed above. In certain particular embodiments, the cancer is breast cancer. In some embodiments, the method further includes includes subjecting the patient to radiotherapy and/or chemotherapy. For example, the patient may be subjected to radiotherapy after being provided MDA-7 and taxotere each at least once. In some embodiments, the patient is subjected to a sub-lethal dose of radiotherapy. In some embodiments, the patient is subjected to resection of all or part of a tumor from the patient. Taxotere may be provided before, during, or after tumor resection. In some embodiments, the patient is provided MDA-7 and/or taxotere at least by administering a composition to the resulting tumor bed. The taxotere may be administered once, or more than once.

In embodiments wherein the composition includes a nucleic acid, the nucleic acid may be in a vector. For example, the vector may be a viral vector. In particular embodiments, the viral vector is an adenoviral vector. In some embodiments, the adenoviral is formulated with protamine. Any number of viral particles may be administered to the patient per administration. In certain embodiments, about 10⁹ to about 10¹³ viral particles are administered to the patient/administration.

In some of the embodiments of the present invention, wherein a nucleic acid composition is administered, the nucleic acid composition may include one or more lipids. Any of the lipids discussed above may be included in these lipid-nucleic acid compositions. Examples of such lipids include DOTAP and cholesterol, or a derivative thereof.

The present invention also generally concerns methods of predicting the efficacy of MDA-7 cancer therapy in a subject that involve: (a) assaying a biological sample that includes cells from the subject for a level of IL-10 expression, and (b) administering or not administering the MDA-7 cancer therapy depending on whether the level of IL-10 expression correlates with MDA-7 resistant cells or MDA-sensitive cells.

In some embodiments, the subject is a subject who has already been treated with chemotherapy, radiotherapy, or some other form of anti-cancer therapy. Any method known to those of ordinary skill in the art can be used to assay a biological sample for a level of IL-10 expression. For example, in some embodiments, the level of IL-10 expression is assayed using an antibody that specifically recognizes IL-10. In other embodiments, the level of IL-10 expression is assayed using a nucleic acid primer or proble that is complementary or identical to the IL-10 transcript.

The biological sample that includes cancer cells can be any type of biological sample, so long as it contains one or more cancer cells. For example, the biological sample may be a body fluid sample, such as a plasma sample, a serum sample, a blood sample, a cerebrospinal fluid sample, or a urine sample. In particular embodiments, the biological sample is a tissue sample, such as a sample of tumor tissue from the subject.

In further embodiments, the method of predicting the efficacy of MDA-7 cancer therapy in a subject further involves providing to the subject an IL-10 inhibitor if the subject expresses a level of IL-10 that correlates with MDA-7 resistant cells. Any of the methods set forth above can be applied in the administration of IL-10 inhibitor to the subject. Furthermore, based on the teachings set forth herein, one of ordinary skill in the art would be able to determine whether the level of IL-10 expressed by the subject correlates with MDA-7 resistant cells.

The present invention is also generally directed to methods for preventing a disease or health-related condition in a patient that include providing MDA-7 to the patient, wherein the MDA-7 is sufficient to prevent cancer in the subject. The disease or health-related condition, for example, may be a premalignant lesion or a cancer. The cancer may be any of those cancers discussed above. As discussed above, the subject may be a subject at risk of developing a cancer. For example, the subject may have a genetic predisposition, or may have a history of successfully treated cancer.

Methods of providing MDA-7 to a subject for prevention of a disease or health-related condition include any of the methods set forth above. In certain embodiments, MDA-7 is provided to the patient by administering to the patient a composition comprising a nucleic acid having a sequence encoding MDA-7 polypeptide, wherein the MDA-7 polypeptide is expressed in the patient. The composition may be a pharmaceutically acceptable composition. As discussed above, the discussion of which is incorporated into this section, the nucleic acid may be in a vector, such as a viral vector. Administration can be by any method known to those of ordinary skill in the art, such as any of those methods discussed above.

The present invention also concerns methods for preventing cancer in a patient that include administering to the patient an adenovirus vector that includes a nucleic acid sequence encoding MDA-7, wherein the nucleic acid sequence is under the control of a promoter capable of being expressed in the patient. Adenovirus vectors are discussed in detail above, the discussion of which is incorporated into this section. Administration can be by any method known to those of ordinary skill in the art, and include those methods discussed above.

In some embodiments, the patient has a history of cancer that has been successfully treated with chemotherapy, radiotherapy, chemotherapy, immunotherapy, and/or gene therapy. In some more particular embodiments, the patient is subjected to radiotherapy. For example, the patient may be subjected to radiotherapy after being provided with MDA-7 and the MDA-7 conjunctive agent at least once. The dose of radiotherapy may be any dose known to those of ordinary skill in the art. For example, in some embodiments, the dose is a sublethal dose of radiotherapy.

The present invention also generally pertains to methods for treating a premalignant lesion in a patient that include providing MDA-7 to the patient. Providing MDA-7 can be by any method known to those of ordinary skill in the art, such as by administering to the patient a composition that includes a nucleic acid having a sequence encoding MDA-7 polypeptide, wherein the MDA-7 polypeptide is expressed in the patient. The composition may be a pharmaceutically acceptable composition, as discussed above. In certain embodiments, the nucleic acid is in a vector. Vectors are as discussed above, the discussion of which is incorporated into this section.

The composition may be formulated for administration as discussed herein, including oral, intravenous, and direct injection of the composition.

Any embodiment discussed with respect to one aspect of the invention applies to other aspects of the invention as well. For instance, any embodiment discussed in the context of one MDA-7 conjunctive agent may be applied to any other MDA-7 conjunctive agent.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B. Cell viability after celecoxib, Ad-mda7 or Ad-mda7 plus celecoxib treatment for 72 hours. HER2− (MDA-MB-436) (a) and HER2+ (MCF7/Her18) (b) breast cancer cells were treated with PBS (phosphated buffered saline) as control, Ad-luc (luciferase) as reporter, Ad-mda7, Celecoxib and combination of Ad-mda7 and celecoxib (M+C). The combination showed most significantly decreased survival fraction compared to control (PBS). The viability was measured by MTT assay. The absorbance is plotted as percentage viability against control. (*p<0.05)

FIG. 2. Cell death determination by trypan blue exclusion after 72-hour treatment. Her2− (MDA-MB-436) and Her2+ (MCF7/Her18) breast cancer cells were treated with Ad-mda7, Ad-luc, celecoxib or combination of Ad-mda7 and celecoxib (M+C) for 3 days and cell viability was assessed by trypan blue exclusion. Both cell lines showed profoundly increased dead cell population in combined group (M+C) compared to control (PBS). Data are plotted as % cell death versus treatment. (*p<0.05)

FIG. 3A-B. Cell cycle analysis after 72-hour treatment. Her2− (MDA-MB-436) (a) and Her2+ (MCF7/Her18) (b) breast cancer cells were treated with Ad-mda7, Ad-luc, celecoxib or combination of Ad-mda7 and celecoxib for 3 days, and then floating and adherent cells were gathered for the cell cycle analysis. MCF7/Her18 cells showed significantly increased G1 phase of cell cycle in combination (M+C) compared to control (PBS).

FIG. 4A-B. Flow cytometry by Annexin V/FITC and TUNEL assay. MDA-MB-436 (a) and MCF7/Her18 (b) cells were harvested after 72-hr treatment, and then stained according to the manufacturer's protocol. By Annexin V/FITC assay, celecoxib and mda7 treatment showed increased apoptosis (p<0.05), and the combined treatment of Ad-mda7 and celecoxib (M+C) showed the most increased percentage of apoptosis to all groups (p<0.05). By TUNEL assay, combined and celecoxib treatment showed significant increase of apoptosis compared to control (PBS) (p<0.05). (*p<0.05)

FIG. 5A-B. Enhancement of adenoviral mda-7 mediated cell killing by geldanamycin (GA) in human lung cancer cells. (A) Percentage of cell death in A549 and H460 cells following treatment with different doses of geldanamycin. The cells were analyzed by flow cytometry 48 h after treatment. Triplicate experiments were performed for each cell line. (B) Flow cytometric analysis of apoptosis in A549 and H460 cells after Ad-mda7, Ad-luc, GA, Ad-mda7 plus GA and Ad-luc plus GA treatment. Triplicate experiments were performed for each cell line.

FIG. 6A-B. Ad-mda7 and GA inhibit lung cancer cell motility. (A) Flow cytometry analysis of surface E-cadherin levels in A549 and H460 cells after treated with PBS, Ad-luc, Ad-mda7, Ad-mda7 plus GA, Ad-luc plus GA and GA. Triplicate experiments were performed for each cell line. (B) Lung cancer cell motility was determined as described in the “Materials and Methods”. Ad-mda7 plus GA markedly reduced the motility of A549 and H460 lung cancer cells. The data shown are representative of three independent experiments.

FIG. 7. 17AAG enhance adenoviral mda-7 mediated cell killing in human lung cancer cells. Flow cytometric analysis of apoptosis in A549 and H460 cells after Ad-mda7, Ad-luc, 17AAG, Ad-mda7 plus 17AAG and Ad-luc plus 17AAG treatment. Triplicate experiments were performed for each cell line.

FIG. 8A-B. Ad-mda7 treatment in combination with Vitamin E succinate inhibits growth of human ovarian cancer cells but does not inhibit growth of normal cells. (A) Human ovarian cancer cells (MDAH 2774) or (B) normal human fibroblast cells (MRC-9) were treated with Ad-luc (vector control), Tocopherol (Vitamin E succinate, 8 μg/mL), Ad-mda7 (2000 vp/cell) or a combination thereof.

FIG. 9. Western blot analysis using antibodies against Fas was performed on MDAH 2774 cells were treated with Ad-luc (vector control), Tocopherol (Vitamin E succinate, 8 μg/mL), Ad-mda7 (1000 vp/cell) or a combination thereof. The level of Fas protein present under each treatment condition was quantitated plotted on the Y axis as the percentage increase in Fas protein as compared to untreated cells.

FIG. 10A-C. DOTAP:Chol-mda-7 complex suppresses growth of subcutaneous tumors. Subcutaneous tumor-bearing (A549 or UV223m) nude mice and C3H mice were divided into groups and treated daily for a total of six doses (50 μg/dose), as follows: no treatment, PBS, DOTAP:Chol-LacZ complex or DOTAP:Chol-CAT complex, and DOTAP:Chol-mda-7 complex. (A) A549. (B) UV2237m. Each time point represents the mean tumor volume for each group. Bars represent standard errors. (C) Subcutaneous tumors were harvested 48 hours after treatment and analyzed for MDA-7 protein expression. In tumors treated with the DOTAP:Chol-mda-7 complex, 18% of A549 tumor cells and 13% of UV2237m tumor cells produced the MDA-7 protein, while control tumors produced no MDA-7 protein.

FIG. 11. MDA-7 induces apoptotic cell death following treatment with the DOTAP:Chol-mda-7 complex. Subcutaneous tumors (A549, and UV2237m) from animals receiving no treatment, PBS, DOTAP:Chol-LacZ or DOTAP:Chol-CAT complex, or DOTAP:Chol-mda-7 complex were harvested and analyzed for apoptotic cell death by TUNEL staining. The percentages of cells undergoing apoptotic cell death (13% for A549 and 9% for UV2237m) in tumors treated with DOTAP:Chol-mda-7 complex were significantly higher (P=0.001) than in the other treatment groups. Bars denote standard deviation.

FIG. 12. DOTAP:Chol-mda-7 complex inhibits tumor vascularization. Subcutaneous tumors (A549, and UV2237m) that were either untreated or treated with PBS, DOTAP:Chol-LacZ or DOTAP:Chol-CAT complex, or DOTAP:Chol-mda-7 complex were stained for CD31 and subjected to semi-quantitative analysis. CD31-positive endothelial staining was significantly lower (P=0.01) in DOTAP:Chol-mda7-treated tumors than in the tumors of other treatment groups. Bars denote standard deviation.

FIG. 13. DOTAP:Chol-mda-7 complex inhibits experimental lung metastases. Lung tumor (A549, UV2237m)-bearing nu/nu or C3H mice were treated daily for a total of six doses (50 μg/dose) with PBS, DOTAP:Chol-CAT complex or DOTAP:Chol-mda-7 complex. Metastatic tumor growth was significantly inhibited (P=<0.05) in both nude mice and C3H mice that were treated with DOTAP:Chol-mda-7 complex compared with that in the two control groups. Bars denote standard deviation.

FIG. 14. Chemosensitization of ovarian cancer cells. Cells treated with Ad-luc and Taxol or Ad-mda7 and Taxol showed growth inhibition compared to other treatment groups. However, significant growth inhibition that was additive to synergistic was observed only in cells that were treated with Ad-mda7 and Taxol (P=<0.05). Experiments were conducted in triplicate wells and the results represented as the average of two separate experiments. Bars denote standard error.

FIG. 15. Ad-mda7 selectively inhibits growth of breast tumor cells but not normal cells. Summary of cell lines, p53 mutational status (m: mutated; wt: wild type) and IC₅₀ (concentration of vector required for 50% growth inhibition by tritiated thymidine assay) values are shown for Ad-mda7 compared to control vector (*: either Ad-luc or Ad-empty). #.: selectivity index (S.I) assessed as the ratio of IC₅₀ for Ad-mda7 divided by IC₅₀ for Ad-control.

FIG. 16A-E. MDA-7 induces PKR and is cytotoxic to tumor cells. (A) Western blot analysis of MCF-7 and MDA-MB-453 cells treated with Ad-mda7 (mda-7) or Ad-luc (luc) at a MOI of 2000 vp/cell. MDA-7 protein was present in Ad-mda7-treated but not control-treated cells. PKR protein is induced by MDA-7. β-actin is a control to demonstrate equal protein loading. (B) Treatment of breast carcinoma cells with Ad-mda7 inhibits tumor cell growth in vitro. Tritiated thymidine assays were performed in T47D; MDA-MB-361; MCF-7; BT-20; cells were treated with Ad-mda7 or Ad-luc (0-10,000 vp/cell; 0-500 pfu/cell) for 4 days and growth monitored by ³H-thymidine uptake. Data are shown as mean+SD. (C) MDA-7 induces G2/M cell arrest (arrow). Tumor cells transduced with Ad-mda7, Ad-luc, or vehicle control were analyzed for cell cycle using PI and flow cytometry. (D) Ad-mda7 induces tumor cell death in a dose- and time-dependent manner. MDA-MB-453 cells transduced with Ad-mda7 or Ad-luc and analyzed by Trypan Blue staining 24-72 h post transduction. Results are shown as percent cell death versus vector dose and time. Data are plotted as mean+SD. (E) Ad-mda7 does not induce cell death in HMEC cells. The cells were transduced with Ad-mda7 or Ad-luc and analyzed by trypan blue staining after 4 days. The results are presented as percent cell death versus dose (0-10,000 vp/cell). Data are plotted as mean+SD.

FIG. 17A-C. Ad-mda7 induces apoptosis in breast cancer cells. (A) Breast tumor lines treated with Ad-mda7 or Ad-luc for 3 days and apoptosis analyzed using Annexin V. *p<0.01. Data are plotted as mean+SD. (B) MDA-7 induces BAX in T47D cells. T47D cells were treated with 2000 vp/cell od Ad-luc or Ad-mda7 and lysates evaluated for MDA-7 and BAX expression. Treatment with ZVAD reduced apoptosis but not MDA-7 or BAX. (C) MDA-7 induces apoptosis-related proteins in breast carcinoma cells. MDA-MB-468 cells were treated with Ad-mda7, Ad-luc or no treatment (UT). Cell lysates were probed with antibodies against caspase 3, PARP, and MDA-7. Beta-actin (β-actin) is used as an internal control to demonstrate equal protein loading.

FIG. 18A-D. Ad-mda7 inhibits growth of breast tumor xenografts. Breast cancer cells: MCF-7 (A), MDA-MB-468 (B), and MDA-MB-361 (C) were used to induce tumor formation in nude mice. The tumors were then injected with Ad-mda7, Ad-luc or PBS and their growth followed. The results are shown as tumor volume (in mm³) versus time (in days). *p<0.002 for MCF-7 and MB-361; p<0.004 for MB-468. (D) Ad-mda7 induces apoptosis in MDA-MB-468 breast cancer xenografts. Tumors were established in nude mice and injected with PBS, Ad-Luc, or Ad-mda7, harvested 24 hours later, and fixed in formalin. Paraffin embedded sections were subjected to immunohistochemical analysis with antibodies against MDA-7 protein or PKR protein. In the Ad-mda7 treated tumors, significant increase in MDA-7 and PKR expression (blue stained cells) was observed. Ad-mda7 treated tumors also showed high levels of TUNEL signals (arrows). Control tumors did not show MDA-7 expression, PKR induction or apoptosis.

FIG. 19. Ad-mda7 significantly inhibits breast tumor growth in multiple models. Tumor models and p53 status are indicated.

FIG. 20A-B. Ad-mda7 is synergistic when combined with Tamoxifen. (A) Growth inhibition of T47D cells treated with Ad-mda7 or Ad-empty in combination with Tamoxifen. Cells treated with Ad vectors (0-1000 vp/cell) and increasing doses of Tamoxifen (0-2 μg/mL) for 3 days, analyzed for cell proliferation. (B) MCF-7 and T47D cells treated with Ad-mda7 or Ad-luc as monotherapy or in combination with 1 μg/ml Tamoxifen. Data are shown as mean+SD.

FIG. 21A-B. Combination treatment with Taxotere and Ad-mda7 inhibit breast cancer cell growth. Breast cancer cell lines (A) T47D and (B) MCF-7 were treated with Ad-mda7 or Ad-luc (0-2000 vp/cell), and Taxotere (0-2 ng/mL), as indicated. After 3 days, proliferation was assayed using ³H-thymidine uptake. Data are shown as mean+SD.

FIG. 22A-D. Combination treatment of Ad-mda7 with Adriamycin or Herceptin inhibits tumor cell growth. (A) T47D and (B) MCF-7 breast carcinoma cell lines treated with Ad-mda7 or Ad-luc and Adriamycin (0-1 ng/mL) as indicated. After 3 days, proliferation was assayed using ³H-thymidine uptake. Data are shown as mean+SD. (C) Western analysis of lysates from MDA-MB-453 cells treated with Ad-mda7 (M) or Ad-luc (L) as monotherapy (control) or in combination with Taxotere, Adriamycin or Herceptin. Blots were probed using antibodies against p53 and BCL-2 family members. Tubulin staining was used to verify equal loading. (D) Ad-mda7 synergizes with Herceptin in Her2+ cells. Cell death was measured by trypan blue staining in MDA-MB-453 (Her2+) and MCF-7 (Her2−) breast tumor cells after 3 days treatment with control (UT); 1 μg/ml Herceptin (H); 2000 vp/cell Ad-luc (L); Ad-mda7 (M) or combinations as indicated. Data are shown as mean+SD.

FIG. 23. Ad-mda7 modulates different apoptotic regulators when combined with chemotherapy. MDA-MB-453 cells treated with Ad-mda7, Ad-luc (2000 vp/cell) or vectors combined with the indicated therapeutic agents. Cell lysates were immunoblotted using antibodies against p53; BCL-2; BCL-XL; BAX and normalized using tubulin. Signals in Ad-mda7 lysates were compared to the corresponding Ad-luc treatment. ↓: decreased expression compared to Ad-luc control; ↑: increased expression compared to Ad-luc control; -: no change between Ad-mda7 or Ad-luc; n.s.: no signal.

FIG. 24A-B. Combination studies with Ad-mda7 and radiotherapy. (A) The combination of Ad-mda7 plus radiation (RT) decreases cell survival in clonogenic assay. MDA-MB-468 breast cancer cells were treated with RT, Ad-empty or Ad-mda7 at 2000 vp/cell; 48 hours after infection, cells were irradiated (0, 2, or 4 Gy), and evaluated by clonogenic assay. The combination of Ad-mda7 plus radiation therapy synergistically inhibited colony formation in breast cancer cells, as compared to control treatment. (B) The combination of radiation therapy (RT) and Ad-mda7 markedly decreases breast cancer growth in vivo. When MDA-MB-468 breast cancer tumors reached approximately 100 mm³, animals were divided into six treatment groups (n=5 animals in each group); PBS, Ad-luc, Ad-luc+RT, RT, Ad-mda7 and Ad-mda7+RT. Recombinant adenoviruses were delivered by intratumoral injection at a dose of 2×10¹⁰ vp/ml every other day for a total of 3 injections. 24 hours after the third injection, a single dose of 5 Gy was delivered to the hind limb. Tumors were assessed for growth by measurements in two dimensions and tumor volume was recorded. There was a marked difference in tumor size between the treatment groups. Ad-mda7 monotherapy produced greater tumor growth inhibition than RT alone or Ad-luc/RT. The most marked growth suppression was seen in the animals that received the combination of XRT and Ad-mda7. *:p<0.002

FIG. 25. Ad-mda7 vector infected breast cancer cells express IL-24 protein resulting in cell killing. MDA-MB231 and MDA-MB453 were transduced with various amount of Ad-mda7 or Ad-luc as indicated for 72 h. Results of cell counting by trypan blue exclusion assay are plotted as mean+SD of two independent experiments using triplicate samples. **p<0.01 compared to Ad-luc.

FIG. 26. Ad-mda7 induces cell cycle arrest and apoptosis. (A) MDA-MB453 cells were transduced with Ad-mda7 or Ad-luc as indicated. Cells were analyzed by FACS assay following 72 h incubation. Figures showed PI stained cell distribution. Arrows indicate G2/M cell population which was significantly higher than control or Ad-luc treated cells (p<0.05). Results are plotted as mean+SD of two independent experiments. (B) 5000 vp/cell of Ad-mda7 or Ad-luc was added to MDA-MB231 and MDA-MB453 cells for 3 days and apoptotic cell percentages were quantified by Annexin V analysis. * indicates apoptotic cell population which were significant higher than control (p<0.05). Results are plotted as mean+SD of three independent experiments.

FIG. 27. IL-24 protein activates phospho-STAT3 and induced cell killing in breast cancer cells. MDA-MB231 and MDA-MB453 breast cancer cells and MeWo melanoma cells (positive control) were treated with 3000 vp/cell Ad-mda7 plus normal mouse IgG or anti-IL24 monoclonal antibody at indicated concentrations. After three days incubation, cell death was plotted against treatment. Data are plotted as mean+SD of two independent experiments using triplicate samples. *p<0.01 compared to Ad-mda7 mediated killing.

FIG. 28A-B. IL-24 mediated killing of breast cancer cells occurs via IL-20R1. (A) MDA-MB231 and MDA-MB453 cells were treated with 30 ng/ml IL-24 alone or in combination with 500 ng/ml indicated antibodies (anti-MDA7, anti-IL-20R1, anti-IL-22R1 or normal mouse IgG) for 96 h. *p<0.01 compared to IL-24 mediated killing. Data are shown as mean+SD of triplicate samples. (B) IL-24 protein induces apoptosis in MDA-MB 453 cells. Human IL-24 protein at various dilutions was added to MDA-MB453 cell culture medium. Western blot of IL-24 is shown in upper panel. After 96 h treatment, cells were collected and TUNEL staining was used to determine the apoptotic cell population.

FIG. 29. IL-10 blocked the killing activity of IL-24. (A) MDA-MB231 and MDA-MB453 cells were treated with 30 ng/ml IL-10, IL-19, IL-20, IL-22 or IL-24 for 96 h. Results of cell counting by trypan blue exclusion assay are plotted as mean+SD of two independent experiments using triplicate samples. *p<0.01 compared to IL-10. (B) MDA-MB231 and MDA-MB453 cells were treated with 30 ng/ml IL-24, or IL-24 with increasing concentrations of IL-10 (0-300 ng/ml) or with denatured boiled IL-10. *p<0.05 indicates significant inhibition of cell death compared to IL-24 alone. Data are showed as mean+SD of two independent experiments using triplicate samples.

FIG. 30A-B. MDA-7/IL-24 inhibits VEGF in lung tumor cells. Lung tumor cells were treated with PBS, Ad-Luc, or Ad-mda7. Cells and culture supernatant were collected at 72 h after treatment and analyzed for exogenous MDA-7 protein expression and VEGF from cell lysate by western blotting and VEGF in supernatant by ELISA. (A) MDA-7 and VEGF expression PBS-Ad-luc- and Ad-mda7-treated cells. β-actin was used as internal loading control. (B) VEGF expression as determined by ELISA and expressed as percent inhibition over PBS.

FIG. 31A-B. MDA-7-mediated VEGF inhibition is independent of tumor cell killing. Tumor cells were treated with PBS, Ad-Luc, or Ad-mda7 (1000 vp/cell). (A) Cells were harvested at various time points after treatment and cell viability determined. No significant tumor cell inhibition was observed among the three treatment groups from 24 h to 72 h. (B) Analysis of culture supernatant at 48 h and 72 h after treatment showed decreased VEGF levels in Ad-mda7-treated supernatant compared to Ad-luc treatment. Inhibition of VEGF by Ad-mda7 was observed to be independent of cell killing as observed in the cell viability assay.

FIG. 32. MDA-7-mediated VEGF inhibition occurs by inhibiting Src. Tumor cells were treated with PBS, Ad-Luc, or Ad-mda7, harvested, and analyzed for expression of Src kinase activity, as described in Example 8. Inhibition of Src kinase by Ad-mda7 was markedly increased compared to the Src activity in PBS and Ad-luc treated cells.

FIG. 33. MDA-7-mediated VEGF inhibition in tumor cells affects endothelial cell proliferation and cell signaling. (A) Conditioned medium from H1299 cells treated with PBS, Ad-luc, or Ad-mda7 were collected at 48 h after treatment and added to HUVECs in the presence or absence of excess anti-MDA7 neutralizing antibody (10 μg/ml), or recombinant human VEGF₁₆₅ protein (50 ng/ml). Cells were analyzed for cell proliferation by trypan blue assay and for VEGFR2 signaling by western blotting as described in Example 8. Conditioned medium from Ad-mda7-treated H1299 cells significantly inhibited HUVEC proliferation compared to conditioned medium from PBS- and Ad-luc treated cells. (B) Analysis for VEGFR2 and pAKT, a downstream target of VEGF receptor signaling in HUVEC at 5, 10 and 60 min showed activation of VEGFR2 and AKT in HUVECs treated with conditioned medium from PBS- and Ad-luc-treated tumor cells. However VEGFR2 and AKT activation were not observed in HUVECs treated with medium from Ad-mda7-treated tumor cells in the presence or absence of anti-MDA7 neutralizing antibody. VEGFR2 and AKT activation in HUVECs were restored when rhVEGF protein was added to the conditioned medium from Ad-mda7-treated tumor cells.

FIG. 34. Bevicizumab but not MDA-7 inhibits endothelial cell proliferation. Tumor (H1299) cell and endothelial (HUVEC) cells were treated with PBS, Ad-luc, Ad-mda7, Avastin, Ad-luc plus Avastin or Ad-mda7 plus Avastin. Three different concentrations of Avastin was tested in combination with Ad-luc or Ad-mda7. Cells were harvested at 48 h and 72 h after treatment and subjected to cell viability by trypan blue exclusion assay. In tumor cells there was no significant inhibition observed in any of the treatment groups. However, in endothelial cells, a significant inhibition was observed in cells treated with Avastin, Ad-luc plus Avastin and Ad-mda7 plus Avastin. However, the most significant inhibition was observed when endothelila cells were treated with Ad-mda7 and Avastin in a dose-dependent manner.

FIG. 35. VEGF inhibition by a combination of MDA-7 and Avastin in tumor cells affects endothelial cell proliferation. Conditioned medium from H1299 cells treated with PBS, Ad-luc, Ad-mda7, Avastin, Ad-luc plus Avastin or Ad-mda7 plus Avastin were collected at 48 h after treatment and added to HUVECs. Cells were analyzed for cell proliferation by trypan blue assay in “Materials and Methods”. Conditioned medium from Ad-mda7-, Avastin-, Ad-luc plus Avastin- and Ad-mda7 plus Avastin treated H1299 cells significantly inhibited HUVEC proliferation compared to conditioned medium from PBS- and Ad-luc treated cells. However, the inhibitory effect most significant when HUVEC were treated with conditioned medium from Ad-mda7 plus Avastin treated tumor cells.

FIG. 36. Ad-mda7 plus Avastin significantly reduced VEGF in vivo.

FIG. 37. MDA-7 plus Avastin inhibits tumor growth in vivo. Subcutaneous H1299 tumors cells were established in nude mice by injecting H1299 tumor cells (5×10⁶). Animals were divided into groups (n=8/group) and treated as follows: PBS, Ad-luc, Ad-mda7, Avastin, Ad-luc plus Avastin, and Ad-mda7 plus Avastin. Ad-mda7 or Ad-luc (1×10¹⁰ vp/injection) was injected intratumorally and Avastin (5 mg/Kg) was injected intraperitoneally. Treatments were given twice a week for four weeks and tumor size measured three times a week. At the end of the experiment animals were euthanized and tumors isolated and subjected to immunohistochemical analyses and western blotting. A significant tumor growth inhibition was observed in mice that were treated with Ad-mda7 plus Avastin compared to other treatment groups. Significant inhibition of tumor growth was also observed in Ad-mda7-, Avastin- and Ad-luc plus Avastin-treated mice compared to tumors from mice treated with PBS or Ad-luc. No significant reduction in body weight was observed in any of the treatment groups (measured till day 28).

FIG. 37. Ad-mda7 plus TNF-alpha treatment inhibits tumor cell proliferation. Prostate tumor (LNCaP) tumor cells were treated with PBS (P), TNF-alpha (T), Ad-Luc (L), Ad-mda7 (M), Ad-luc plus TNF (L+T) or Ad-mda7 plus TNF (M+T). Viral treatment was at 1500 vp/cell and TNF treatment at 5 ng/ml. At 48 h and 72 h after treatment cells were subjected to XTT assay to determine cell viability. Cells treated with Ad-mda7 plus TNF showed significant growth inhibition compared to other treatment groups.

FIG. 38. TNF-alpha increases the transduction efficiency. Tumor (LNCaP) cells were treated with Ad-GFP at 100, 300, 600 and 1200 vp/cell in the presence or absence of TNF-alpha (10 ng/ml). Cells receiving no treatment served as control. At 24 h after TNF-alpha treatment cells were harvested, washed with PBS three times, resuspended in 500 ul PBS and subjected to FACS analysis. Cells treated with Ad-GFP alone showed a dose-dependent increase in transduction efficiency starting from 73.5% for 100 vp/cell of Ad-GFP. However, in the presence of TNF-alpha, the transduction efficiency was increased and was observed to be 92.8% for 100 vp/cell of Ad-GFP. The increase in transduction appeared to be saturated from 300 vp/cell of Ad-GFP in the presence of TNF-alpha.

FIG. 39. Ad-mda7 plus TNF-alpha treatment results in increased number of cells in SubG0/G1 phase. Tumor (LNCaP) cells were treated with PBS (P), TNF-alpha (T; 10 ng/ml), Ad-Luc (L; 1500 vp/cell), Ad-mda7 (M; 1500 vp/cell), Ad-luc plus TNF (L+T), Ad-mda7 plus TNF (M+T), Ad-luc plus anti-TNF antibody (L+A; 1 ug/ml) or Ad-mda7 plus anti-TNF antibody (M+A). At 48 h after treatment cells were harvested, washed three times with PBS, resuspended in 500 ul of PBS containing propidium iodide (0.5 ug/ml). Cells were subjected to FACS analysis. A significant number of cells treated with Ad-mda7 plus TNF was observed in the SubG0/G1 phase (70%)-indicated apoptotic cells compared to other treatment groups that ranged from 0.45% to 26.3%.

### DESCRIPTION OF ILLUSTRATIVE EMBODIMENTS

### A. MDA-7 Compositions

The compositions and methods of the present invention employ MDA-7 polypeptides and nucleic acids encoding such polypeptides. MDA-7 is a tumor suppressor that has been shown to suppress the growth of cancer cells that are p53-wild-type, p53-null and p53-mutant. Also, the observed upregulation of the apoptosis-related B gene in p53 null cells indicates that MDA-7 is capable of using p53-independent mechanisms to induce the destruction of cancer cells.

### B. MDA-7

Mda-7 mRNA has been identified in human PBMC (Ekmekcioglu et al., 2001), and no cytokine function of human MDA-7 protein was reported. MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine (Denkert et al., 2004). The rat MDA-7 homolog C49a (Mob-5) is 78% homologous to the mda-7 gene and has been linked to wound healing (Soo et al. 1999; Zhang et al., 2000). Mob-5 was also shown to be a secreted protein and a putative cell surface receptor was identified on ras transformed cells (Zhang et al., 2000). Therefore, homologues of the MDA-7 gene and the secreted MDA-7 protein are expressed and secreted in various species. However, no data has emerged to show MDA-7 has cytokine activity. Such activity has ramifications for the treatment of a wide variety of diseases and infections by enhancing immunogenicity of an antigen.

The human mda-7 cDNA (SEQ ID NO:1) encodes an evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. A combination of structural data, homology to known cytokines, chromosomal localization, a predicted N-terminus secretion signal peptide, and evidence of its regulation of cytokine secretion, all support classification of MDA-7/IL-24 as a IL-10 family cytokine (see Chada et al., 2004 review). A 49 amino acid leader sequence identifies it as a secreted protein; recent studies confirm this and report that Ad-mda7 transduced cells release high levels of a 40 kDa form of the MDA-7 protein, which can bind to heterodimeric receptors IL-20R1/IL-20R2 and IL-22R2/IL-20R1. The intracellular form of the protein (23-30 kDa) is cleaved, and extensively modified (primarily by glycosylation) before its release into the extracellular compartment (see Chada et al., 2004 review, which is hereb incorporated by reference).

The expression of MDA-7 is inversely correlated with melanoma progression as demonstrated by increased mRNA levels in normal melanocytes as compared to primary and metastatic melanomas as well as decreased MDA-7 expression in early vertical growth phase melanoma cells selected for enhanced tumor formation in nude mice. Reports indicate that MDA-7 is an IL-10 family cytokine with tumor cell apoptotic activity and that the cytotoxic effects it induces are specific to tumor cells (see Chada et al., 2004 review). Several studies have investigated the signal transduction pathways that mediate the apoptotic activity of mda-7. These appear to be multiple, cell-type specific, and include effects induced by the intracellular form of the protein, and by the secreted form (bystander effect) (see U.S. Ser. No. 10/791,692, which is incorporated by reference). Additional information and data regarding MDA-7 can be found in U.S. patent application Ser. Nos. 09/615,154, 10/017,472, 10/378,590, and 10/791,692, all of which applications are herein incorporated by reference in their entireties.

Additional studies have shown that elevated expression of MDA-7 suppressed cancer cell growth in vitro and selectively induced apoptosis in human breast cancer cells as well as inhibiting tumor growth in nude mice (Jiang et al., 1996 and Su et al., 1998). Jiang et al. (1996) report findings that MDA-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2). MDA-7 overexpression in normal cells (HMECs, HBL-100, and CREF-Trans6) showed limited growth inhibition indicating that mda-7 transgene effects are not manifest in normal cells. Taken together, the data indicates that growth inhibition by elevated expression of MDA-7 is more effective in vitro in cancer cells than in normal cells.

Su et al. (1998) reported investigations into the mechanism by which MDA-7 suppressed cancer cell growth. The studies reported that ectopic expression of MDA-7 in breast cancer cell lines MCF-7 and T47D induced apoptosis as detected by cell cycle analysis and TUNEL assay without an effect on the normal HBL-100 cells. Western blot analysis of cell lysates from cells infected with adenovirus mda-7 (“Ad-mda7”) showed an upregulation of the apoptosis stimulating protein BAX. Ad-mda7 infection elevated levels of BAX protein only in MCF-7 and T47D cells and not normal HBL-100 or HMEC cells. These data lead the investigators to evaluate the effect of ex vivo Ad-mda7 transduction on xenograft tumor formation of MCF-7 tumor cells. Ex vivo transduction resulted in the inhibition of tumor formation and progression in the tumor xenograft model.

The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growing tumor with necessary nutrients. Endostatin and angiostatin are examples of two such therapies (WO 00/05356 and WO 00/26368).

Though not adhering to a particular theory regarding the operability of these constructs, there is a notable amino acid homology of mda-7 to IL-10 and across species in the D-helical region, located at the C-terminus, which is implicated in receptor binding. Thus, molecules preferably containing this 30-35 amino acid region are particularly preferred.

Thus, in one embodiment of the present invention, the treatment of angiogenesis-related disease involves the administration of a therapeutic peptide or polypeptide. In another embodiment, treatment involves administration of a nucleic acid expression construct encoding mda-7 to target, comprising diseased cells or endothelial cells. It is contemplated that the target cells take up the construct, and express the therapeutic polypeptide encoded by nucleic acid, thereby inhibiting differentiation in the target cells. Cells expressing MDA-7 in turn can secrete the protein which may interact with neighboring cells not transduced or infected by an expression construct. In this way the complex interactions needed to establish new vasculature for the tumor is inhibited and treatment of the tumor accomplished.

In another embodiment of the present invention, it is contemplated that an angiogenesis-related disease may be treated with a MDA-7, or constructs expressing the same. Some of the angiogenesis-related diseases contemplated for treatment in the present invention are psoriasis, rheumatoid arthritis (RA), inflammatory bowel disease (IBD), osteoarthritis (OA) and pre-neoplastic lesions in the lung.

In yet another embodiment, the treatment of a wide variety of cancerous states is within the scope of the invention. For example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment. In particular embodiments, the cancer involves a tumor, which may or may not be resectable. Moreover, the cancer may involve metastatic tumor(s) or a tumor possibly capable of metastasis.

Cancer cells that may be treated by methods and compositions of the invention also include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments of the present invention, the mda-7 is provided as a nucleic acid expressing the MDA-7 polypeptide. In specific embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles. In certain embodiments, the viral vector is an adenoviral vector, a retroviral vector, a vaccinia viral vector, an adeno-associated viral vector, a polyoma viral vector, an alphaviral vector, a rhabdoviral vector, or a herpesviral vector. Most preferably, the viral vector is an adenoviral vector. In other specific embodiments, the nucleic acid is a non-viral vector.

In certain embodiments, the nucleic acid expressing the polypeptide is operably linked to a promoter. Non-limiting examples of promoters suitable for the present invention include a CMV IE, dectin-1, dectin-2, human CD11c, F4/80, SM22 or MHC class II promoter, however, any other promoter that is useful to drive expression of the mda-7 gene or the immunogene of the present invention, such as those set forth herein, is believed to be applicable to the practice of the present invention.

Preferably, the nucleic acid of the present invention is administered by injection. Other embodiments include the administering of the nucleic acid by multiple injections. In certain embodiments, the injection is performed local, regional or distal to a disease or tumor site. In some embodiments, the administering of nucleic acid is via continuous infusion, intratumoral injection, intraperitoneal, or intravenous injection. In other embodiments, the nucleic acid is administered to the tumor bed prior to or after; or both prior to and after resection of the tumor. Alternatively, the nucleic acid is administered to the patient before, during, or after chemotherapy, biotherapy, immunotherapy, surgery or radiotherapy. Preferably the patient is a human. In other embodiments the patient is a cancer patient.

### C. Nucleic Acids, Vectors and Regulatory Signals

The present invention concerns polynucleotides or nucleic acid molecules relating to the mda-7 gene and its gene product MDA-7. Additionally, the present invention is directed to polynucleotides or nucleic acid molecules relating to an immunogenic molecule. These polynucleotides or nucleic acid molecules are isolatable and purifiable from mammalian cells. It is contemplated that an isolated and purified MDA-7 nucleic acid molecule, either the secreted or full-length version, that is a nucleic acid molecule related to the mda-7 gene product, may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding MDA-7” refers to a nucleic acid segment that contains MDA-7 coding sequences, yet is isolated away from, or purified and free of, total genomic DNA and proteins. When the present application refers to the function or activity of a MDA-7-encoding polynucleotide or nucleic acid, it is meant that the polynucleotide encodes a molecule that has the ability to induce apoptosis of a cancer cell.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). There may be times when the full or partial genomic sequence is some. Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode an MDA-7 polypeptide. In particular cases, a human MDA-7 polypeptide is a specific embodiment. Consequently, the present invention also encompasses derivatives of MDA-7 with minimal amino acid changes, but that possess the same activity.

The term “gene” is used for simplicity to refer to a functional protein, polypeptide, or peptide-encoding nucleic acid unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding MDA-7 may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO:1 (MDA-7 encoding sequence).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a MDA-7 protein, polypeptide or peptide that includes within its amino acid sequence a contiguous amino acid sequence in accordance with, or essentially as set forth in, SEQ ID NO:2, corresponding to the MDA-7 designated “human MDA-7” or “MDA-7 polypeptide.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2” provided the biological activity of the protein is maintained with respect to inducing apoptosis. In particular embodiments, the biological activity of a MDA-7 protein, polypeptide or peptide, or a biologically functional equivalent, comprises enhancing an immune response. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:2. Again, DNA segments that encode proteins, polypeptide or peptides exhibiting MDA-7 activity will be employed in embodiments of the invention.

In particular embodiments, the invention concerns isolated nucleic acid segments and recombinant vectors incorporating DNA sequences that encode MDA-7 polypeptides or peptides that include within its amino acid sequence a contiguous amino acid sequence in accordance with, or essentially corresponding to MDA-7 polypeptides.

Vectors of the present invention are designed, primarily, to transform cells with a therapeutic mda-7 gene or MDA-7 encoding nucleic acid sequence under the control of a eukaryotic promoter (i.e., constitutive, inducible, repressable, tissue specific). Also, the vectors may contain a selectable marker if, for no other reason, to facilitate their manipulation in vitro. However, selectable markers may play an important role in producing recombinant cells.

Tables 1 and 2, below, list a variety of regulatory signals for use according to the present invention.

TABLE 1
Inducible Elements

| Element | Inducer | References |
|---|---|---|
| MT II | Phorbol Ester (TPA) | Palmiter et al., 1982; Haslinger and |
| | Heavy metals | Karin, 1985; Searle et al., 1985; |
| | | Stuart et al., 1985; Imagawa et al., |
| | | 1987; Karin et al., 1987; Angel et |
| | | al., 1987b; McNeall et al., 1989 |
| MMTV (mouse | Glucocorticoids | Huang et al., 1981; Lee et al., 1981; |
| mammary tumor | | Majors and Varmus, 1983; Lee et |
| virus) | | al., 1984; Ponta et al., 1985 |
| β-Interferon | poly(rI) × poly(rc) | Tavernier et al., 1983 |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | Ela, SV40 Large T | Taylor et al., 1989; Taylor and |
| | Antigen | Kingston, 1990a, b |
| Proliferin | Phorbol Ester-TPA | Mordacq and Linzer, 1989 |
| Tumor Necrosis | MA | Hensel et al., 1989 |
| Factor | | |
| Thyroid | Thyroid Hormone | Chatterjee et al., 1989 |
| Stimulating | | |
| Hormone α Gene | | |

TABLE 2
Other Promoter/Enhancer Elements

| Promoter/Enhancer | References |
|---|---|
| Immunoglobulin Heavy Chain | Banerji et al., 1983; Gillies et al., 1983; |
| | Grosschedl and Baltimore, 1985; Atchinson and |
| | Perry, 1986, 1987; Imler et al., 1987; Neuberger |
| | et al., 1988; Kiledjian et al., 1988; |
| Immunoglobulin Light Chain | Queen and Baltimore, 1983; Picard and Schaffner, |
| | 1985 |
| T-Cell Receptor | Luria et al., 1987, Winoto and Baltimore, 1989; |
| | Redondo et al., 1990 |
| HLA DQ α and DQ β | Sullivan and Peterlin, 1987 |
| β-Interferon | Goodbourn et al., 1986; Fujita et al., 1987; |
| | Goodbourn and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; |
| | Johnson et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, |
| | 1989 |
| γ-Globin | Bodine and Ley, 1987; Perez-Stable and |
| | Constantini, 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell Adhesion Molecule | Hirsch et al., 1990 |
| (NCAM) | |
| a₁-Antitrypain | Latimer et al., 1990 |
| H2B (TH2B) Histone | Hwang et al., 1990 |
| Mouse or Type I Collagen | Rippe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh |
| | and Lockett, 1985; Firak and Subramanian, 1986; |
| | Herr and Clarke, 1986; Imbra and Karin, 1986; |
| | Kadesch and Berg, 1986; Wang and Calame, |
| | 1986; Ondek et al., 1987; Kuhl et al., 1987 |
| | Schaffner et al., 1988 |
| Polyoma | Swartzendruber and Lehman, 1975; Vasseur et al., |
| | 1980; Katinka et al., 1980, 1981; Tyndell et al., |
| | 1981; Dandolo et al., 1983; Hen et al., 1986; |
| | Campbell and Villarreal, 1988 |
| Retroviruses | Kriegler and Botchan, 1983; Kriegler et al., |
| | 1984a, b; Bosze et al., 1986; Miksicek et al., 1986; |
| | Celander and Haseltine, 1987; Thiesen et al., |
| | 1988; Celander et al., 1988; Chol et al., 1996; |
| | Reisman and Rotter, 1989 |
| Papilloma Virus | Campo et al., 1983; Lusky et al., 1983; Spandidos |
| | and Wilkie, 1983; Spalholz et al., 1985; Lusky |
| | and Botchan, 1986; Cripe et al., 1987; Gloss et |
| | al., 1987; Hirochika et al., 1987, Stephens and |
| | Hentschel, 1987 |
| Hepatitis B Virus | Bulla and Siddiqui, 1988; Jameel and Siddiqui, |
| | 1986; Shaul and Ben-Levy, 1987; Spandau and |
| | Lee, 1988 |
| Human Immunodeficiency Virus | Muesing et al., 1987; Hauber and Cullan, 1988; |
| | Jakobovits et al., 1988; Feng and Holland, 1988; |
| | Takebe et al., 1988; Berkhout et al., 1989; Laspia |
| | et al., 1989; Sharp and Marciniak, 1989; |
| | Braddock et al., 1989 |
| Cytomegalovirus | Weber et al., 1984; Boshart et al., 1985; Foecking |
| | and Hofstetter, 1986 |
| Gibbon Ape Leukemia Virus | Holbrook et al., 1987; Quinn et al., 1989 |

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In some embodiments, the promoter for use in the present invention is the cytomegalovirus (CMV) immediate early (IE) promoter. This promoter is commercially available from Invitrogen in the vector pcDNAIII, which is some for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

Compositions and methods of the invention are provided for administering the compositions of the invention to a patient.

### D. Vectors

An MDA-7 polypeptide may be encoded by a nucleic acid molecule comprised in a vector. In this manner, an MDA-7 polypeptide can be provided to a patient through the administration of such a vector, so long as the polypeptide is expressed in the patient.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (2001) and Ausubel et al., 1996, both incorporated herein by reference. In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Viral Vectors

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

c. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Protamine

Protamine may also be used to form a complex with an expression construct. Such complexes may then be formulated with the lipid compositions described above for adminstration to a cell. Protamines are small highly basic nucleoproteins associated with DNA. Their use in the delivery of nucleic acids is described in U.S. Pat. No. 5,187,260, which is incorporated by reference. U.S. patent application Ser. No. 10/391,068 (filed Mar. 24, 2003), which pertains to methods and compositions for increasing transduction efficiency of a viral vector by complexing the viral vector with a protamine molecule, is specifically incorporated by reference herein.

2. Non-Viral Delivery

In addition to viral delivery of the nucleic acid encoding a MDA-7 protein, the following are additional methods of recombinant gene delivery to a given host cell and are thus considered in the present invention.

a. Lipid Mediated Transformation

In a further embodiment of the invention, an expression vector may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

Manufacture and use of such a formulation for the treatment of cancer is provided in 09/575,473, which is hereby incorporated by reference.

In further embodiments, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some embodiments, the nanoparticles are extruded during the production process. Exemplary information pertaining to the production of nanoparticles can be found in U.S. Patent App. Pub. No. 20050143336, U.S. Patent App. Pub. No. 20030223938, U.S. Patent App. Pub. No. 20030147966, and U.S. Ser. No. 60/661,680, each of which is herein specifically incorporated by reference into this section.

In certain embodiments, an anti-inflammatory agent is administered with the lipid to prevent or reduce inflammation secondary to administration of a lipid:nucleic acid complex. For example, the anti-inflammatory agent may be a non-steroidal anti-inflammatory agent, a salicylate, an anti-rheumatic agent, a steroid, or an immunosuppressive agent. Information pertaining to administration of anti-inflammatory agents in conjunction with lipid-nucleic acid complexes can be found in U.S. Patent App. Pub. No. 20050143336, which is herein specifically incorporated by reference.

Synthesis of DOTAP:Chol nanoparticles is by any method known to those of ordinary skill in the art. For example, the method can be in accordance with that set forth in Chada et al., 2003, or Templeton et al., 1997, both of which are herein specifically incorporated by reference. DOTAP:Chol-DNA complexes were prepared fresh two to three hours prior to injection in mice.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these liposomes include colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

The liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

A nucleic acid for nonviral delivery may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

### E. Proteins, Peptides and Polypeptides

The present invention is directed to methods and compositions of MDA-7 polypeptides. In certain embodiments, the MDA-polypeptides are used in the treatment of cancer. In certain embodiments, the MDA-7 polypeptide is directly provided. The terms “protein” and “polypeptide” are used interchangeably herein.

Additional embodiments of the invention encompass the use of a purified protein composition comprising MDA-7 protein and a truncated version of MDA-7 lacking its endogenous signal sequence or an MDA-7 polypeptide with a heterologous signal sequence. Truncated molecules of MDA-7 include, for example, molecules beginning approximately at MDA-7 amino acid residues 46-49 and further N-terminal truncations. Specifically contemplated are molecules start at residue 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206. In additional embodiments, residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48 are included with other contiguous residues of MDA-7, as shown in SEQ ID NO:2.

The present invention is also directed to methods and compositions of MDA-7 or nucleic acids encoding MDA-7 in combination with one or more of the following: (a) a TNF, (b) a VEGF inhibitor, or (c) an IL-10 inhibitor. In certain embodiments of the present invention, the TNF, VEGF inhibitor, or IL-10 inhibitor is a protein, polypeptide, or peptide.

As will be understood by those of skill in the art, modification and changes may be made in the structure of a MDA-7 polypeptide or peptide, TNF polypeptide or peptide, VEGF inhibitor polypeptide or peptide, or IL-10 inhibitor and still produce a molecule having like or otherwise desirable characteristics. For example, certain amino acids may be substituted for other amino acids or include deletions, additions, or truncations in the protein sequence without appreciable loss of interactive binding capacity with structures. Since it is the interactive capacity and nature of a protein that defines that protein's biological functional activity, certain amino acid sequence substitutions can be made in a protein sequence (or, of course, its underlying DNA coding sequence) and nevertheless obtain a protein with similar tumor suppressive, apoptosis-inducing, antiogenic, or cytokine properties. It is thus contemplated by the inventors that various changes may be made in the sequence of MDA-7 polypeptides or peptides (or underlying DNA) without appreciable loss of their biological utility or activity. The full-length amino acid and nucleic acid sequences of TNF-alpha are attached herein as SEQ ID NO:3 and SEQ ID NO:4, respectively. The full-length amino acid and nucleic acid sequences of TNF-beta are attached herein as SEQ ID NO:5 and SEQ ID NO:6, respectively.

VEGF-A exists in several isoforms derived from a single gene by alternative splicing. The full-length amino acid sequence and nucleic acid sequence of isoform 121 of VEGF-A are set forth herein as SEQ ID NO:7 and SEQ ID NO:8, respectively. The full-length amino acid sequence and nucleic acid sequence of isoform 165 of VEGF-A are set forth herein as SEQ ID NO:9 and SEQ ID NO:10, respectively. The full-length amino acid sequence and nucleic aid sequence of isoform 189 of VEGF-A are set forth herein as SEQ ID NO:11 and SEQ ID NO:12, respectively. The full-length amino acid sequence and nucleic acid sequence of isoform 206 of VEGF-A are set forth herein as SEQ ID NO:13 and SEQ ID NO:14, respectively. The full-length amino acid sequence and nucleic acid sequence of VEGF-B are set forth herein as SEQ ID NO:15 and SEQ ID NO:16, respectively. The full-length amino acid sequence and nucleic acid sequence of VEGF-C are set forth herein as SEQ ID NO:17 and SEQ ID NO:18, respectively. The full-length amino acid sequence and nucleic acid sequence of VEGF-D are set forth herein as SEQ ID NO:19 and SEQ ID NO:20, respectively. The full-length amino acid sequence and nucleic acid sequence of placental growth factor, a member of the VEGF family, are set forth herein as SEQ ID NO:21 and SEQ ID NO:22, respectively.

In terms of functional equivalents, the skilled artisan also understands it is also well understood by the skilled artisan that inherent in the definition of a biologically-functional equivalent protein or peptide, is the concept of a limit to the number of changes that may be made within a defined portion of a molecule that still result in a molecule with an acceptable level of equivalent biological activity. Biologically-functional equivalent peptides are thus defined herein as those peptides in which certain, not most or all, of the amino acids may be substituted. In particular, where small peptides are concerned, less amino acids may be changed. Of course, a plurality of distinct proteins/peptides with different substitutions may easily be made and used in accordance with the invention.

It is also well understood that where certain residues are shown to be particularly important to the biological or structural properties of a protein or peptide, e.g., residues in the active site of an enzyme, or in the RNA polymerase II binding region, such residues may not generally be exchanged.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, some, and those within ±0.5 are even more particularly preferred.

It is also understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, some, those which are within ±1 are particularly preferred, some, and those within ±0.5 are even more particularly preferred some.

While discussion has focused on functionally equivalent polypeptides arising from amino acid changes, it will be appreciated that these changes may be effected by alteration of the encoding DNA, taking into consideration also that the genetic code is degenerate and that two or more codons may encode the same amino acid.

1. In Vitro Protein Production

In addition to the purification methods provided in the examples, general procedures for in vitro protein production are discussed. Following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshney, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogene product, and more specifically, an protein having immunogenic activity. Other examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

2. ER-Targeting Sequences

The polypeptides of the present invention include one or more endoplasmic reticulum targeting sequences. The final location of a protein within a cell depends upon targeting sequences encoded within the sequence of a protein. In the simplest case, the lack of a signal directs proteins to the default pathway which is the cytoplasm. Proteins destined to be retained in the ER must have certain signal peptides to retain the protein in the ER. The polypeptides of the present invention may or may not include additional amino acid residues at the N-terminal or C-terminal.

The ER is a network of membrane-enclosed tubules and sacs (cisternae) that extends from the nuclear membrane throughout the cytoplasm. The secretory pathway of proteins is as follows: rough ER→Golgi→secretory vesicles→cell exterior.

For proteins to be secreted, the protein must generally travel from the ER to the Golgi. However, there are certain proteins that must be maintained within the ER, such as BiP, signal peptidase, protein disulfide isomerase. Specific localization signals target proteins to the ER.

Certain proteins are retained in the ER lumen as a result of the presence of the ER targeting sequence Lys-Asp-Glu-Leu (KDEL, in the single-letter code) at their carboxy terminus. If this sequence is not part of the protein, the protein is instead transported to the Golgi and secreted from the cell. The presence of the KDEL sequence or the KKXX sequence at the carboxy terminus (KKXX sequences) results in retention of proteins in the ER. The presence of these sequences results in binding of the protein to specific recycling receptors in the membranes of these compartments and are then selectively transported back to the ER.

Protein export from the ER occurs not only by bulk flow, but by a regulated pathway that specifically recognizes targeting signals that mediate selective transport of proteins to the Golgi apparatus. The presence of a 16- to 30-residue ER signal sequence directs the ribosome to the ER membrane and initiates transport of the protein across the ER membrane.

ER signal sequences are usually located at the N-terminus of the protein. These targeting sequences frequently contains one or more positively charged amino acids followed by a continuous stretch of 6-12 hydrophobic residues. Signal sequences are usually cleaved from the protein while it is still growing on the ribosome. The specific deletion of several of the hydrophobic amino acids from a signal sequence or a mutation of one of them to a charged amino acid results in failure of the protein to cross the ER membrane into the lumen. The addition of random N-terminal amino acid sequences will cause a cytosolic protein to be translocated to the ER lumen, indicating that the hydrophobic residues form a binding site that is critical for ER targeting.

The endoplasmic reticulum targeting sequence may include any number of amino acid residues, as long as these amino acid residues target the destination of the polypeptide to the endoplasmic reticulum. The polypeptides of the present invention may include a single ER targeting sequence, or more than one ER targeting sequence. Additional information pertaining to ER targeting signals can be found in Invitrogen Catalog Nos. V890-20, V891-20, V892-20, and V893-20, “pShooter Vector Manual I (pEF/myc vectors),” on the internet at invitrogen.com/content/sfs/manuals/pshooter_pef_man.pdf, which is hereby incorporated by reference in its entirety. Reviews of signal sequence recognition and protein targeting to the ER can also be found in Walter and Johnson, 1994; Koch et al., 2003; and Kabat et al., 1987, which are also specifically incorporated by reference herein.

3. Methods of MDA-7 Purification

The present invention employs purified MDA-7 in some embodiments of the invention. The following methods and similar methods known to one of ordinary skill in the art can be used to practice the methods of purification of MDA-7 disclosed herein. Such methods are disclosed in 10/791,692, which is hereby incorporated by reference. Part of this disclosure is provided below (without figures).

### F. Antibody Production

1. Antibodies that Bind MDA-7

Recombinant his-tagged MDA-7 protein was produced in E. coli and was purified on a nickel NTA agarose column. The material was bound to the nickel resin in a batch mode for 45 minutes and then poured into a column and the eluate was run through the column bed. The material was washed with 10 mM Tris pH 8.0 containing 0.5% chaps and finally eluted off of the column with 10 mM Tris pH 8.0 plus 400 mM imidazole. The eluted MDA-7 was dialyzed against 10 mM Tris pH 8.0. The final product was shown to be a single band with a molecular weight of approx. 23 kDa. The amino terminal protein sequence was shown to be correct and purity was estimated to be greater than 90%.

This material was injected into rabbits using the following protocol: 400 mg MDA-7 protein with IFA and 100 mg of MDP was injected subcutaneously, 3 weeks later 200 μg MDA-7 protein with IFA was injected and 3 weeks after that another 100 mg of MDA-7 protein was injected intravenously. The titer of antiserum was shown to be greater than 1/100,000 based on an ELISA assay. Animals were boosted as needed.

The MDA-7 protein was coupled via sulfhydryl linkage to a solid support resin. The resin and bound protein was thoroughly washed. This washed material was used to make an MDA-7 column for antibody purification. The rabbit polyclonal sera was diluted 1:1 with 20 mM Tris buffer pH 8.0 and filtered through a 0.2-micron filter before being pumped onto the MDA-7 column. The column was then washed with the same 20 mM Tris buffer pH 8.0 until the absorbance returned to baseline. The antibody was eluted off the column with 0.1 M acetic acid. The eluent containing the antibody was immediately adjusted back to pH 8.0. This affinity-purified antibody was then dialyzed against 10 mM Tris pH 8.0 and concentrated.

2. Antibodies that Bind IL-10 and VEGF

Some embodiments of the present invention pertain to methods and compositions involving MDA-7 in combination with an inhibitor of IL-10, wherein the inhibitor is an antibody. The present invention also concerns methods and compositions involving MDA-7 in combination with a VEGF-inhibitor wherein the VEGF inhibitor is an antibody that binds VEGF.

Information pertaining to antibodies that bind to immunomodulators, such as IL-10, can be found in U.S. Pat. No. 6,168,791, wherein is herein specifically incorporated by reference. U.S. Pat. No. 6,168,791 teaches antibodies and methods for production of antibodies that bind to immunomodulators, such as IL-10 or an agonist of IL-10. Additional information regarding IL-10 antibody production can be found in U.S. Pat. No. 6,407,218 and U.S. Patent Application 20050101770, each of which is herein specifically incorporated by reference. Moreover, a discussion below regarding antibodies for MDA-7 purification may be implemented in the context of a VEGF or IL-10-specific antibody.

Examples of IL-10 antibody sequences include an antibody molecule that binds IL-10 or binding fragment thereof, including: at least one antibody light chain variable region, or binding fragment thereof, comprising a polypeptide having at least one amino acid sequence selected from the group consisting of at CDR1 (SEQ ID NO:23), SEQ ID NO:24 at CDR2, and SEQ ID NO:25 at CDR3; and a framework region, wherein the amino acid sequence of framework region is all or substantially all of a human immunoglobin amino acid sequence; and at least one antibody heavy chain variable region, or binding fragment thereof, comprising a polypeptide having at least one amino acid sequence selected from the group consisting of SEQ ID NO:26 at complementarity determining region 1 (CDR1), SEQ ID NO:27 at CDR2, and SEQ ID NO:28 at CDR3; and a framework region, wherein the amino acid sequence of framework region is all or substantially all of a human immunoglobin amino acid sequence. The antibody may further include a heavy chain constant region, wherein the heavy chain constant region comprises a gamma-1, gamma-2, gamma-3, or gamma-4 human heavy chain constant region or a variant thereof. The antibody may further include a light chain constant region, wherein the light chain constant region comprises a lambda or a kappa human light chain constant region.

Additional information regarding anti-human-IL-10 antibodies can be found on the world wide web at sigmaaldrich.com/sigma/datasheet/i5020dat.pdf, which is hereby incorporated by reference.

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Included within the definition of an antibody that binds IL-10 is an IL-10 antibody binding fragment. As used herein, the term “IL-10 binding fragment” or “binding fragment thereof” encompasses a fragment or a derivative of an antibody that still substantially retain its biological activity of inhibiting IL-10 activity. Therefore, the term “antibody fragment” or IL-10 binding fragment refers to a portion of a full length antibody, generally the antigen binding or variable region thereof. Examples of antibody fragments include Fab, Fab′, F(ab′).sub.2, and Fv fragments; diabodies; linear antibodies; single-chain antibody molecules, e.g., sc-Fv; and multispecific antibodies formed from antibody fragments. Typically, a binding fragment or derivative retains at least 50% of its IL-10 inhibitory activity. Preferably, a binding fragment or derivative retains at least 60%, 70%, 80%, 90%, 95%, 99% or 100% of its IL-10 inhibitory activity. It is also intended that a IL-10 binding fragment can include conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Any suitable method for generating monoclonal antibodies may be used. For example, a recipient may be immunized with IL-10 or a fragment thereof. Any suitable method of immunization can be used. Such methods can include adjuvants, other immunostimulants, repeated booster immunizations, and the use of one or more immunization routes.

Any suitable source of IL-10 or VEGF can be used as the immunogen for the generation of the non-human antibody of the compositions and methods disclosed herein. Such forms include, but are not limited whole protein, peptide(s), and epitopes, generated through recombinant, synthetic, chemical or enzymatic degradation means known in the art.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

### G. Purification and Characterization of Secreted MDA-7 Using Polyclonal Antibodies

1. Affinity Column Production

Different polyclonal antibodies against human MDA-7 from rabbit serum were first purified. Frozen rabbit serum samples were thawed and diluted 1:1 with sterile 1×PBS buffer. The diluted samples were individually exposed in bath method at 4° C. overnight with gentle rocking to 2 mls Protein A-Sepharose (SIGMA). Four different columns were generated. The resin was washed with 10 column volumes of 20 mM sodium phosphate dibasic (61 mls) to make a pH of 7.0 The column was eluted with 3 column volumes of 0.15 M NaCl (pH 3.0) in three aliquots and neutralized with 0.5M HEPES. A Bradford Protein Assay (BioRad) was used to quantify the eluted antibody. The antibody was then exchanged into 0.1 M NaHCO₃ (pH 8.3) containing 0.5 M NaCl, by dialyzing overnight in a 10,000 MWCO dialysis cassette.

To activate the dried CNBr-Sepharose, 1 gram was washed with 10-15 column volumes with 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. In each case, approximately 80-90 milligrams of antibody was recovered after purification and buffer exchange. Then 5 mls of swollen activated CNBr-Sepharose was incubated with 80-90 milligrams of purified antibody in 0.1 M NaHCO₃, pH 8.3, for 4 hours at room temperature with gentle rotation.

Antibody binding efficiency was determined by Bradford Protein assay, and in each case was greater than 95% of the antibody bound to the activated CNBr-Sepharose. After coupling, non-reacted groups were blocked by washing 25-30 column volumes in 0.1 M Tris, pH 8.0. The column was then washed with serial washes of 0.1 M Tris, pH 8.0, 0.5 M NaCl, 5× column volumes 5 times, alternating with 0.1 M acetate buffer, pH 4.0, 0.5 M NaCl. Protein estimation was performed on the washes and no protein was detected.

2. Affinity Chromatography Purification

Stably transfected 293 T cells that secrete soluble, glycosylated MDA-7 were obtained and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycine, (20 mg/ml stock). Then 400 mls of supernatant was harvested every 2-3 days and concentrated with an AMICON stirred cell over a 10,000 molecular weight cutoff membrane. 50 mls of concentrated supernatant was exposed in batch method to 5 mls bed volume of antibody-CNBr-sepharose, (affinity resin) for 2 days at 4° C. with gentle rocking. The affinity resin was then placed in a Pharmacia XK 26 column and the supernatant passed through three times to ensure maximum binding of antigen to antibody. The affinity resin was washed with 5×20 mls 0.1 M Tris pH 8.0 by gravity flow. MDA-7 was eluted with 3×5 mls 1 M NaCl, 0.1 M Glycine, pH 3.0 and immediately neutralized with 0.5 mls HEPES buffer. Immediately after elution and neutralization, 2 mgs of human albumin was added to protect against protein loss. The eluted protein was then concentrated over 10,000 molecular weight cutoff spin columns (AMICON), and exchanged into sterile 1×PBS. Then 1-1.5 mls of 1×PBS exchanged affinity purified protein was exposed to 200 microliters 3× washed Protein-A Sepharose (SIGMA) for 2 hours at room temperature with rotation, or over night at 4° C. with rotation. Protein A exposure absorbs antibody that leaches into the elution fraction.

Four different polyclonal antibodies, whose production is described herein, were tested in affinity purification. Size resolution purification (see Size Exclusion) was employed to removed significant contaminating protein from the supernatant prior to affinity purification, the most abundant of which was bovine serum albumin (BSA). However, exposure of MDA-7 isolated in this fashion failed to permit the antibody on the column to retain MDA-7. This was probably due to BSA blocking non-specific binding sites that could retain MDA-7 in the absence of BSA. MDA-7 is a highly glycosylated protein it is considered very capable of sticking to plastic and other surfaces.

Removal of BSA from MDA-7 containing supernatant inhibits purification of MDA-7 by affinity chromatography. Most protein was present in the flow through. No MDA-7 protein is retained on the affinity column until elution. Affinity purifications that contained significant amounts of BSA, (2-3 mgs/ml by silver stain) retained biological function for longer than the purifications wherein the BSA contamination was significantly less. Affinity purification in the presence of BSA permits the retention of MDA-7 on the affinity column until elution with high molar NaCl and low pH. Affinity purification by polyclonal affinity resin resulted in multiple lots with relatively similar amounts of MDA-7. Coomassie analysis indicated relatively low quantities of contaminating protein. Purification of MDA-7 of greater than about 20% homogeneity was observed.

Affinity purification was repeatable and enriched the MDA-7 to relative purity by coomassie stain analysis of 12% polyacrylamide gels. By intensity of bands detected on the Western blot, more MDA-7 was retained with longer exposure of the antigen to the affinity resin. There was little difference between the method of exchange into 1×PBS, when comparing the dialysis cassette and the spin columns.

3. Anion Exchange Purification

Two to three lots of affinity purified MDA-7 were pooled and exchanged into 50 mM MES, pH 5.0 in a 10,000 MWCO dialysis cassette from 2-12 hrs at room temperature. Protein was then loaded onto a 5 ml bed volume anion exchange column at a flow rate of 1 ml/minute. 10 mls of flow through were taken and the bound protein was eluted with a step gradient of 1 M NaCl in 50 mM MES, pH 5.0. The elution program began with a 10 ml wash of 50 mM MES, pH 5.0 at flow rate of 2 mls/min. The first step elution was from 0 M to 0.25 M NaCl in 5 minutes with a 5 minute wash at 50 mM MES, 0.25 M NaCl, pH 5.0. The second gradient step was from 0.25 M NaCl to 0.5 M NaCl in 5 minutes followed by a 5 minute wash. The final elution was from 0.5 M NaCl to 1 M NaCl. MDA-7 was retained on to column until elution with 0.9-1.0 M NaCl; MDA-7 was purified to about 90%-95% homogeneity.

The unglycosylated protein of 18 KDa did not bind to the anion exchange column at pH 5.0. Silver stain analysis of fractions from post-affinity anion exchange of MDA-7 revealed that the unglycosylated form of MDA-7 is not associated with the co-purifying glycosylated proteins. The native MDA-7 complex appears to contain at least three proteins of molecular weight 31, 28 and 27/26. Previously, an attempt was made to purify MDA-7 utilizing a one step anion exchange purification, wherein the supernatant containing MDA-7 was exchanged into 50 mM MES, pH 6.0. One step anion exchange purification demonstrated that each peak from the anion exchange column contains MDA-7 detected by polyclonal anti-MDA-7 on western blot. Purification by this method failed to significantly enrich for MDA-7 at any range of ionic strength, as MD A-7 leached from the column at all molarities of NaCl employed.

4. Size Exclusion Chromatography

A 200 ml bed volume size exclusion chromatography column was generated utilizing S200 Sephadex (Pharmacia) poured into an XK 26 1 meter column (Pharmacia). The column was allowed to gravity settle, and was then packed at 3.5 mls/min with a BioRad BioLogic Workstation.

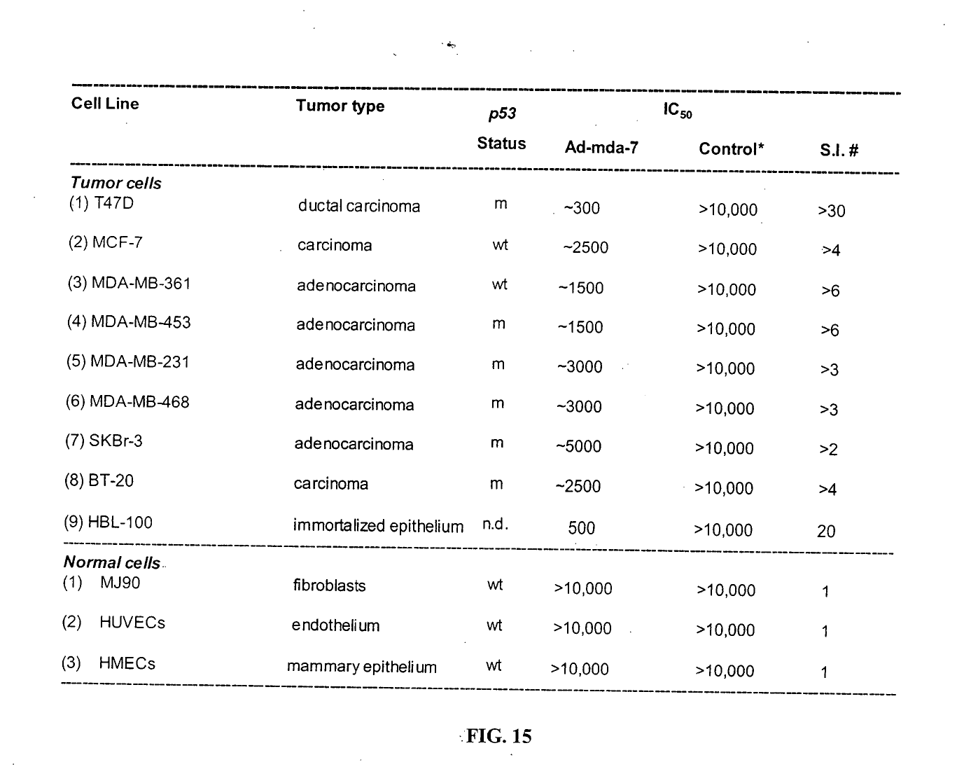

To determine the apparent molecular weight of MDA-7 secreted by the 293 t cells, protein molecule weight standards, (mouse IgG 5 mgs, Alkaline Phosphatase 3 mgs, BSA 10 mgs, and human beta2 microglobulin 3 mgs) were combined to determine the relative retention times. Elution times of the purified proteins relative to molecular weights were plotted and an R² value of 0.97 derived. 200 mls of 293 t supernatant containing MDA-7 was concentrated over a 10,000 MWCO filter in an AMICON stirred cell down to 10 mls and loaded at 2 mls/min in 1×PBS on the size resolution column. Fractions were taken every 5 mls. Relative retention times was determined by Western blot analysis of sequential samples and compared to the line derived from the known standards. An apparent molecular weight of 80-100 kDa was assigned to the associated MDA-7. Less than 0.1% of the total MDA-7 present was found to be in monomeric 31 kDa form. FIG. 15 shows a comparison of retention time to molecular weight. MDA-7 complex was eluted at between a molecular weight of about 85-95 kDa.

5. Size, Anion, and Lectin Purification

Lectin purification over a ConcanavalinA-Sepharose column was employed in an attempt to purify MDA-7. However, no net increase in relative purity was achieved. Combinatorial purifications, wherein size exclusion, anion, and lectin purification methods, were utilized in all combinations to enrich for MDA-7. However, no combination of these methods provided for greater purification of MDA-7 than affinity chromatography followed by anion chromatography. These results demonstrate that MDA-7 can be purified to at least 90-95% homogeneity by affinity and anion exchange chromatography.

### H. Purification and Characterization of Secreted MDA-7 Using Monoclonal Antibodies

1. Antibody Production

The hybridoma clone, designated 7G11F.2 (monoclonal antibody), was determined to produce antibody that was the most effective at detecting IL-24/mda-7 positive cells by intracellular FACS analysis of stably transfected 293t cells that had been treated with Brefeldin A. Based upon these preliminary data, this clone was utilized to produce 5 liters of supernatant. Briefly cells, (7G11F.2) were seeded at 1×10⁶ cells/ml in 50 mls of DMEM supplemented with containing 10% Fetal Calf Serum with 1:100 L-glutamine, 1:100 pen/strep and 1:100 HEPES. Cells were seeded and permitted to grow for 10 days, then the supernatant was harvested.

2. Antibody Purification

Supernatant was clarified of cells by centrifugation at 2000 rpm for 10 minutes and decanted. The clarified supernatant was then sterile filtered over a 0.22 micro cellulose acetate filter and concentrated with an Amicon Stirred Cell under nitrogen over a YMCO 30 kDa membrane to 50 mls. The concentrated supernatant was exposed to rProtein G cross-linked to sepharose, (Sigma) o/n at 4° C. The antibody was eluted with 1 M NaCl pH 3.0, 3 column volumes in three aliquots and neutralize with 0.5 M HEPES. To remove contaminating bovine IgG, the resulting eluate was exchanged into 1×PBS containing 0.4 M NaCl (total), via dialysis cassette (Pierce/Endogen, YMCO 30 kDa). The protein was exposed to rProtein A crosslinked to sepharose, (Sigma) o/n 4° C. The flow through from the column was taken, as the protein A binds the bovine IgG with higher affinity than the mouse IgG1a. Relative purity was determined by analysis on SDS PAGE and taken to be 90% pure, (7G11F.2) with the contaminating protein wholy comprised of bovine IgG. Bradford Protein Assay, (BioRad), was used to quantify eluted antibody. The antibody was then exchanged into 0.1 M NaHCO₃, pH 8.3 containing 0.5 M NaCl, by dialyzing overnight in a 10,000 MWCO dialysis cassette.

3. Affinity Column Production

To activate dried CNBr-Sepharose, 1 gram was washed with 10-15 column volumes of 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. 25 mgs of antibody, (7G11F.2) was recovered after purification and buffer exchange. 2 mls of swollen, activated CNBr-Sepharose was incubated with the purified antibody in 0.1 M NaHCO3, pH 8.3 for 4 hours at room temperature with gentle rotation.

Antibody binding efficiency was determined by Bradford Protein Assay; greater than 95% of the antibody bound to the activated CNBr-Sepharose.

After coupling, non-reacted groups were blocked by washing 25-30 column volumes in 0.1 M Tris pH 8.0. Finally the column was washed with serial washes of 0.1 M Tris pH 8.0, 0.5 M NaCl, 5× column volume 5 times alternating with 0.1 M acetate buffer, pH 4.0, 0.5 M NaCl. Protein estimation was performed on the washes and no protein was detected.

4. Affinity Purification

Stably transfected 293t cells that secrete soluble, glycosylated IL-24 were obtained from Introgen, Inc. and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycine, (20 mg/ml stock). 400 mls of supernatant is harvested every 2-3 days and concentrated with an Amicon stirred cell over a 10,000 molecular weight cutoff membrane. 50 mls of concentrated supernatant is exposed in batch method to 5 mls bed volume of antibody-CNBr-sepharose, (affinity resin) for 2 days at 4° C. with gentle rocking. The affinity resin was placed in a Pharmacia XK 26 column and the supernatant passed through three times to ensure maximum binding of antigen to antibody. The affinity resin was washed with 5×20 mls 0.1 M Tris pH 8.0 by gravity flow. IL-24 was eluted with 3×5 mls 1 M NaCl, 0.1 M Glycine, pH 3.0 and immediately neutralized with 0.5 mls HEPES buffer. Immediately after elution and neutralization, 2 mgs of Human Albumin was added to protect against protein loss. The eluted protein was then concentrated over 10,000 molecular weight cutoff spin columns, (Amicon) and exchanged into sterile 1×PBS. 1-1.5 mls of 1×PBS exchanged affinity purified protein was exposed to 200 microliters 3× washed rProtein-A Sepharose, (Sigma) for 2 hours at room temperature with rotation, or overnight at 4° C. with rotation. Protein A exposure absorbed antibodies that leached into the elution and its removal is crucial for maintaining IL-24 function.

The 7G11F.2 monoclonal antibody column retained similar amounts of IL-24/mda-7 as the polyclonal columns in the previous section.

The following general techniques are also well known and can be used to implement purification methods.

a. Gel Electrophoresis

Gel electrophoresis is a well-known technique that can be used in the purification procedure. Agarose, agarose-acrylamide or polyacrylamide gel electrophoresis using standard methods (Sambrook et al., 2001) can be utilized in the purification process.

b. Chromatographic Techniques

Alternatively, chromatographic techniques may be employed to effect isolation and purification of MDA-7. There are many kinds of chromatography which may be used in the present invention: adsorption, affinity, partition, ion-exchange and molecular sieve, and many specialized techniques for using them including column, paper, thin-layer and gas chromatography (Freifelder, 1982).

c. Immunological Reagents

Certain aspects of the claimed invention involve use of immunological reagents. In certain embodiments of the claimed invention, immunological reagents are used in the purification of preparations of MDA-7. Antibodies are contemplated for use with purification methods. Such antibodies can be readily created and/or are readily available.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The methods for generating monoclonal antibodies (MAbs) is well known to those of skill in the art.

### I. NSAIDs and COX-2 Inhibitors

NSAIDs are anti-inflammatory agents that are not steroids. In addition to anti-inflammatory actions, they have analgesic, antipyretic, and platelet-inhibitory actions. They are used primarily in the treatment of chronic arthritic conditions and certain soft tissue disorders associated with pain and inflammation. They act by blocking the synthesis of prostaglandins by inhibiting cyclooxygenase, which converts arachidonic acid to cyclic endoperoxides, precursors of prostaglandins. The present invention contemplates the use of those NSAIDS that selectively inhibit the enzyme COX-2.

NSAIDs induce apoptosis in both colon tumor cell lines and animal tissues, and appear to inhibit Ki-ras activation in tumors; however, the activation of Ki-ras has not yet been investigated as a mechanism of NSAID-mediated cytotoxicity. It also is not known if such cytotoxicity is dependent on the anti-inflammatory properties of the NSAIDs. The NSAID sulindac, which also inhibits Ki-ras activation, is metabolized to two different molecules which differ in their ability to inhibit COX, yet both are able to exert chemopreventive effects via the induction of apoptosis. However, Sulindac sulfone lacks COX-inhibitory activity, and it most likely facilitates the induction of apoptosis in a manner independent of prostaglandin synthesis. Again, the present invention involves COX-2 selective inhibitors, which refers to a compound or agent that inhibits cyclooxygenase specifically and directly.

COX-2 selective inhibitors include celecoxib (CELEBREX™), rofecoxib (VIOXX™), valdecoxib (BEXTRA™), lumiracoxib (PREXIGE™), or etoricoxib (ARCOXIA™). The commercial versions of celecoxib and rofecoxib were recently withdrawn because of concerns regarding their effects on the cardiovascular system and a perceived increased risk of cardiovascular disease. PREXIGE and ARCOXIA have not yet been approved by the FDA for use in the United States, though they have been approved in other countries.

1. Celecoxib

In certain embodiments, the present invention is concerned with the COX-2 inhibitor celecoxib. Sold by Searle under the trade name CELEBREX™, celecoxib is chemically designated as 4-[5-(4-methylphenyl)-3-(trifluoromethyl)-1H-pyrazol-1-yl]benzenesulfonamide. The empirical formula is C₁₇H₁₄F₃N₃O₂S, and the molecular weight is 381.38. CELEBREX™ is marketed in 100 or 200 mg oral capsules.

Celecoxib exhibits anti-inflammatory, analgesic and antipyretic activities in animal models. The mechanism of action is thought to be the result of inhibition of prostaglandin synthesis. The enzyme cyclooxygenase-2, or “COX-2,” is an important enzyme in this pathway. Selective inhibition of COX-2 (the related enzyme COX-1 is not inhibited) is a characteristic of celecoxib, and is believed to reduce potential gastrointestinal toxicities associated with inhibition of COX-1.

a. Pharmacokinetics

Peak plasma levels of celecoxib are roughly 3 hours after an oral dose. When taken with a high fat meal, plasma levels were delayed about 1-2 hours, with an increase in total absorption of 10-20%. Aluminum or magnesium containing antacids resulting in a decrease in plasma concentrations. Celecoxib is highly protein bound with the clinical dose range, with in vitro studies indicating that albumin and alpha₁-acid glycoprotein being the major bound species. Cytochrome P450 2C9 is the major metabolizing enzyme of celecoxib. The three primary metabolites are the alcohol, the corresponding carboxylic acid and its glucuronide conjugate; these metabolites are inactive as COX-1 and COX-2 inhibitors. Following a single dose, 57% of the dose was excreted in feces, and 27% in the urine. The effective half-life is roughly 11 hours under fasted conditions.

b. Patient Populations

Geriatric patients had high maximal serum concentrations, and elderly male had high concentrations than elderly females. For elderly patients of less than 50 kg, lower doses should be used initially. Blacks show higher serum concentrations than Caucasians. Hepatic insufficiency increases serum concentration, while renal insufficiency decreases concentration.

c. Drug Interactions

Patients should be questioned regarding the use of drugs that inhibit cytochrome P450 2C9. Specific potential drug interactions include fluconazole and lithium, and possibly furosemide and ACE inhibitors.

d. Side Effects and Contraindications

Side effects for NSAIDs typically include gastroduodenal and gastrointestinal irritation. However, celecoxib shows far less of these effects than other NSAIDs. Other possible side effects include anaphylactoid reactions, although none have been reported for celecoxib. It also should be avoided for patients with advanced renal disease and pregnant mothers.

e. Combinations of NSAIDs

Combinations of various COX-2 inhibitors also may be used for according to the present invention. For example, by using lower doses of multiple COX-2 inhibitors and MDA-7 it is possible to reduce the side effects or toxicities associated with higher doses of individual compounds. Specifically for the purposes outlined in this invention, celecoxib can be used in combination with other COX-2 inhibitors in this manner.

2. VIOXX™

In certain embodiments, the present invention is concerned with the COX-2 inhibitor rofecoxib. Sold by Merck under the trade name VIOXX™, reofecoxib is chemically designated as 4-[4-(methylsulfonyl)phenyl]-3-phenyl-2-(5H)furanone The empirical formula is C₁₇H₁₄O₄S, and the molecular weight is 314.36. VIOXX™ is marketed in 12.5, 25, or 50 mg oral capsules or as an oral suspension with a concentration of 12.5 or 25 mg/5 ml.

Celecoxib exhibits anti-inflammatory, analgesic and antipyretic activities in animal models. The mechanism of action is thought to be the result of inhibition of prostaglandin synthesis by selectively inhibiting COX-2 (and not COX-1).

a. Pharmacokinetics

Peak plasma levels of rofecoxib are 2-3 hours after an oral dose. Food did not affect plasma levels except that peak levels were delayed 1-2 hours after a high fat meal. It is metabolized primarily through reduction by cytosolic enzymes.

b. Drug Interactions

Specific potential drug interactions include rifampin, theophylline, and warfarin.

c. Side Effects and Contraindications

A higher incidence of adjudicated serious cardiovascular thromboses has been observed in patient.

3. Valdecoxib

In certain embodiments, the present invention is concerned with the COX-2 inhibitor valdecoxib. Sold by Pfizer under the trade name BEXTRA, celecoxib is chemically designated as 4-(5-methyl-3-phenyl-4-isoxazolyl)benzenesulfonamide. The empirical formula is C₁₆H₁₄N₂O₃S, and the molecular weight is 314.36. BEXTRA is marketed in 10 or 20 mg oral capsules.

Celecoxib has anti-inflammatory, analgesic and antipyretic activities. It is believed to selectively inhibit COX-2, but not COX-1 at amounts found in plasma.

a. Pharmacokinetics

Peak plasma levels of valdecoxib are achieved after about 3 hours. There was no significant effect caused by food, but when taken with a high fat meal, plasma levels were delayed about 1-2 hours.

Valdecoxib is metabolized by the P450 isoenzymes 3A4 and 2C9, as well as non-P450 enzymes through glucuronidation. Drugs that inhibit 3A4 and/or 2C9, such as fluconazole and etoconazole can increas plasma concentrations.

b. Patient Populations

Differences have either not been identified or not studied.

c. Drug Interactions

Patients should be questioned regarding the use of drugs that inhibit cytochrome P450 2C9 and 3A4. Specific potential drug interactions include fluconazole and ketoconazole. Moreover, there is an active metabolite of valdecoxib in human plasma at a concentration of about 10% of that for valdecoxib.

d. Side Effects and Contraindications

Serious skin reactions have been demonstration. Also, gastrointestinal toxicity has been observed.

### J. Hsp90 Inhibitors

The present invention concerns Hsp90 inhibitors, which refer to compounds that directly bind to Hsp90 and have antitumor activity. Hsp90 is a molecular chaperone that is critical for the folding, assembly and activity of a variety of mutated and overexpressed signaling proteins that promote the growth and/or survival of tumor cells. Hsp90 client proteins include mutated p53, Raf-1, Akt, ErbB2 and hypoxia-inducible factor 1α (HIF-1α) (Neckers, 2002).

In certain embodiments, the compounds are natural and synthetic small molecules, such as benzoquinone ansamycins and their analogs. In particular embodiments, the Hsp90 inhibitor is geldanamycin and its analogs and derivatives.

Geldanamycin (GA) is a benzoquinone ansamycin antibiotic produced by Streptomyces hygroscopicus. It has been shown to have antitumor properties, which are believed to be caused by its ability to bind specifically to the heat shock protein Hsp 90, causing the destabilization and degradation of its client proteins (Whitesell et al., 1994; Neckers et al., 1999).

An analog of GA in clinical trials is 17-Allylamino-17-demethoxygeldanamycin (17AAG), which is a less toxic and more stable analog of geldanamycin (GA) (Schulte et al., 1998). Even though 17AAG binding to Hsp90 is weaker than GA, 17AAG displays similar antitumor effects than GA and has a better toxicity profile. Information from a phase I clinical trials demonstrates 17AAG has anti tumor activity that is achieved at concentrations below the maximum tolerated dose (Agnew et al., 2001).

Included in the present invention are targeted versions of GA. For example, Herceptin, the first mAb approved for therapy of solid tumors, targets Her2 and was chosen to target GA to Her2-overexpressing tumors. NCI has reported that such conjugates deliver a more potent selective cytotoxic impact than Herceptin alone. To prepare such conjugates, GA is modified to introduce a latent primary amine (Mandler et al, 2002). After deprotection, this primary amine provides a site for introduction of a maleimide group that enables linkage to proteins (Mandler et al., 2004). A company called InvivoGen has generated a maleimido derivative of geldanamycin, 17-(3-(4-maleimidobutyrcarboxamido)propyl-amido) geldanamycin (GMB-APA-GA), which can readily be conjugated with Herceptin or other mAbs. Other antibodies are contemplated for use with the present invention, as described in the this application in the context of other embodiments.

Geldanamycin analogs and derivatives include, but are not limited to, 17AAG, NSC 255110, NSC 682300, NSC 683661, NSC 683663, 17DMAG, 15-hydroxygeldanamycin, a tricyclic geldanamycin analog (KOSN-1633), methyl-geldanamycinate, 17-formyl-17-demethoxy-18-O,-21-O-dihydrogeldanamycin and 17-hydroxymethyl-17-demethoxygeldanamycin. See Patel et al. (2004); Smith et al. (2004); Tian et al. (2004); Hu et al. (2004); Le Brazadec et al. (2004), which are hereby incorporated by reference.

In certain embodiments, GA or a derivative or analog of GA is given as part of a treatment regimen with Bortezomib, which is a reversible inhibitor of the chymotrypsin-like activity of the 26S proteasome. Doses may about, at least about, or at most about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7.3.8, 3.9, 4.0, 4.1, 4.2, 4.3, 4.4, 4.5, 4.6, 4.7, 4.8, 4.9, 5.0 mg/m² (with respect to tumor size) or mg/day, or any range derivable therein. In particular embodiments, Bortezomib is given intravenously.

In certain embodiments, patients will receive GA or an analaog or derivative as an intravenous infusion at a dose of 300 mg/m² on days 1, 8, and 15 of 28 day cycles. If needed, multiple cycles of therapy are contemplated.

### K. Vitamin E Compounds

The term “vitamin E” refers to a family of eight related, lipid-soluble, antioxidant compounds (alpha (α)-tocopherol, beta (β)-tocopherol, gamma (γ)-tocopherol, delta (δ)-tocopherol α-tocotrienol, β-tocotrienol, γ-tocotrienol, and δ-tocotrienol). The tocopherol and tocotrienol subfamilies are each composed of alpha, beta, gamma, and delta vitamers having unique biological effects. Tocopherols differ from tocotrienols in that they have a saturated phytyl side chain rather than an unsaturated isoprenyl side chain.

Alpha-tocopherol is the only form of vitamin E that is actively maintained in the human body and is thus the most abundant form of vitamin E found in blood and tissue (Traber, 1999). The main function of alpha-tocopherol in humans appears to be its ability to act as an antioxidant (see World Wide Web at lpi.oregonstate.edu/infocenter/vitamins/vitaminE). A synthetic version of vitamin E is available (a chemical mixture composed of 12.5% authentic RRR-α-tocopherol and 87.5% stereoisomers; namely, 7 molecules produced during the manufacturing process that have the same number and types of atoms found in RRR-α-tocopherol linked in the same order but differing in their spatial arrangement) (see “Dietary Reference Intakes for Vitamin C, Vitamin E, Selenium, and Carotenoids,” 2000, and Kline et al., 2003). Thus, natural authentic vitamin E (referred to as RRR-α-tocopherol or d-α-tocopherol) and synthetic vitamin E (referred to as all-rac[emic]-α-tocopherol or dl-α-tocopherol) are not chemically equivalent.

Both authentic RRR-α-tocopherol and synthetic vitamin E can be purchased as acetate or succinate derivatives. These modifications to the chroman head of RRR-α-tocopherol are performed to protect the hydroxyl moiety at the C-6 position from oxidation when exposed to air and must be removed to restore antioxidant potential (Kline et al., 2003). A nonhydrolyzable ether analogue of RRR-α-tocopherol, referred to as α-TEA has been identified (Lawson et al., 2003). It is specifically contemplated that α-TEA can be used in methods and compositions of the invention as a vitamin E analog. The Birringer et al. paper discusses Vitamin E analogs, and is hereby incorporated by reference (Birringer et al., 2003).

Vitamin E compounds are usually produced and made available in esterified form as alpha-tocopheryl acetate or alpha-tocopheryl succinate. Neither of these forms has any antioxidant activity until converted to alpha-tocopherol in the body by removal of the acetate or succinate moiety in the intestine. The esterified forms are more resistant to oxidation and much more stable with respect to storage time and temperature than the unesterified forms. Activation of the succinate form is slower than the acetate form, but the succinate form appears to access and benefit areas of the tissues that are unavailable to the other forms.

Vitamin E has more than one mechanism in the body. It destroys free radicals (antioxidant function), stabilizes membranes, inhibits the synthesis of prostaglandins and prevents platelet aggregation, induces cell differentiation in some cancer cells (melanoma cells in vitro) and inhibits the growth of some tumor cells (murine neuroblastoma, rat glioma and human prostate). Its cancer-fighting ability may be related to its inhibition of prostaglandin synthesis because an excess of prostaglandins can suppress the immune system (see World Wide Web at springboard4health.com/notebook/v-e.html).

Several studies have described potent anti-tumor activity of RRR-α-tocopheryl succinate (vitamin E succinate; VES), a hydrolyzable ester derivative of RRR-α-tocopherol. Prasad and Edwards-Prasad were the first to describe the capacity of vitamin E succinate but not other forms of vitamin E to induce morphological alterations and growth inhibition of mouse melanoma B-16 cells and to suggest that vitamin E succinate might be a useful tumor therapeutic agent (Prasad et al., 1982). Additional studies have demonstrated that vitamin E succinate is a potent growth inhibitor of a wide variety of epithelial cancer cell types, including breast, prostate, lung, and colon; as well as, hematopoietic-lymphoid leukemia and lymphoma cells, in vitro (Fariss et al., 1994; Kline et al., 1998; Kline et al., 2001; Neuzil et al., 2000; Prasad et al., 1992; Schwartz et al., 1992). Recent studies have demonstrated vitamin E succinate to have anti-tumor activity in animal xenograft and allograft models when administered intraperitoneally (i.p.) (Malafa et al., 2000; Malafa et al., 2002; Neuzil et al., 2001; Weber et al., 2002), suggesting a possible therapeutic potential. Vitamin E succinate administered i.p. or orally (p.o.) has also been shown to have inhibitory effects on carcinogen [benzo(a)pyrene]-induced forestomach carcinogenesis in mice, suggesting potential as an anti-carcinogenic agent (Wu et al., 2001). Investigations have demonstrated that vitamin E succinate-induces concentration- and time-dependent inhibition of cancer cell growth via DNA synthesis blockage, induction of cellular differentiation, and induction of apoptosis (Kline et al., 1998; Kline et al., 2001; Neuzil et al., 2001; You et al., 2001; You et al., 2002; Yu et al., 2001).

Vitamin E succinate is noteworthy not only for its induction of growth inhibitory effects on tumor cells but also for its lack of toxicity toward normal cells and tissues (Fariss et al., 1994; Kline et al., 1998; Kline et al., 2001; Neuzil et al., 2000; Prasad et al., 1992; Schwartz et al., 1992; Weber et al., 2002). The use of a non-hydrolyzable vitamin E succinate derivative has shown that it is the intact compound and not either of its cleavage products (namely, RRR-α-tocopherol or succinic acid), that are responsible for the anti-proliferative effects (Fariss et al., 1994). Thus, the anti-proliferative actions of this vitamin E derivative are considered to be due to non-antioxidant properties. RRR-α-tocopheryl succinate (VES) is a derivative of RRR-α-tocopherol that has been structurally modified via an ester linkage to contain a succinyl moiety instead of a hydroxyl moiety at the 6-position of the chroman head. This ester linked succinate moiety of RRR-α-tocopherol has been the most potent form of vitamin E affecting the biological actions of triggering apoptosis and inhibiting DNA synthesis. This form of vitamin E induces tumor cells to undergo apoptosis, while having no apoptotic inducing effects on normal cells. The succinated form of vitamin E is effective as an anticancer agent as an intact agent; however, cellular and tissue esterases that can cleave the succinate moiety, thereby converting the succinate form of RRR-α-tocopherol to the free RRR-α-tocopherol, render this compound ineffective as an anticancer agent. RRR-α-tocopherol exhibits neither antiproliferative nor proapoptotic biological activity in cells of epithelial or immune origin.

Vitamin E is commonly found in vegetation and more abundantly in seeds from which tocopherols, in the natural state, are easily absorbed and utilized in humans and animals, wild and domestic. See U.S. Pat. No. 5,179,122, which is hereby incorporated by reference. Processing of foods and feeds by industry for long term storage promotes accelerated degradation of Vitamin E content. To compensate for the loss of natural Vitamin E from food sources, nutritional supplements of natural or synthetic Vitamin E are administered by injection or orally. It is well known that tocopherols are unstable molecules. To improve tocopherol stability, manufacturing processes generally attach an acetate or succinate group to tocopherol, making Vitamin E acetate or succinate (d- or dl-alpha-tocopheryl acetate or succinate). It is well known that the efficacy of the hydrophilic nature of aqueous Vitamin E solutions and dispersions upon enteral absorption of Vitamin E can be demonstrated by increased absorption of hydrophilic Vitamin E by the normal and compromised intestine. It is known in the art that the source of Vitamin E, natural or synthetic, also affects its bioavailability. In the compromised gut, Vitamin E absorption was studied in patients with lipid malabsorption syndromes such as cholestatic liver, and cystic fibrosis. Such patients are unable to absorb Vitamin E or other dietary lipids. When a water soluble form of Vitamin E (d-alpha-tocopheryl polyethylene glycol 1000 succinate, or “TPGS”) was administered orally to such patients, an elevation of blood tocopherol was detected within one week. When the same patients were dosed with tocopherol in vegetable oil, there was no significant increase of tocopherol in the blood, (Traber et al., 1988). Thus, the type of tocopherol, natural or synthetic, and the hydrophilic nature of TPGS can be important in determining the absorption and bioavailability of Vitamin E in humans and animals. The advantage of administering Vitamin E in a water-dispersible formulation was shown by Bateman et al. (1984) in a human clinical study in which Vitamins A, E, and B₂ were formulated into a liquid vehicle (Aqua Biosorb) and encapsulated into soft gelatin capsules which were given orally. In the formulation, B₂ was incorporated into the formulation as a suspension with a particle diameter of <=100 nm. The soft elastic gelatin capsules contained by weight % 20% polysorbate 80, 1% sorbitan monooleate and 79% distilled monoglyceride as the water dispersible base. Bateman demonstrated that the hydrophilic nature of water soluble Vitamin B₂, in addition to the lipid soluble Vitamins A and E in his dosage formulation, showed enhanced absorption. Any of these versions of vitamin E are contemplated for use as part of the invention.

Vitamin E conjugates include, but are not limited to, vitamin E pyroglutamic acid (pyroglutamate) conjugates including vitamin E succinic acid (VESA) pyroglutamate conjugate, vitamin E succinic acid (VESA) polyethylene glycol amine pyroglutamate conjugate, vitamin E amine pyroglutamate conjugate; vitamin E pyrrolidinone conjugates including vitamin E succinic acid (VESA) pyrrolidinone conjugate; vitamin E gentisic acid conjugates including vitamin E gentisic acid conjugate. U.S. Pat. No. 6,858,227, which is hereby incorporated by reference.

### L. Vascular Endothelial Growth Factor Inhibitors

Vascular endothelial growth factor (VEGF) is a highly specific mitogen for vascular endothelial cells. Five VEGF isoforms are generated as a result of alternative splicing from a single VEGF gene. These isoforms differ in their molecular mass and in biological properties such as their ability to bind to cell-surface heparan-sulfate proteoglycans. The expression of VEGF is potentiated in response to hypoxia, by activated oncogenes, and by a variety of cytokines. VEGF induces endothelial cell proliferation, promotes cell migration, and inhibits apoptosis. In vivo VEGF induces angiogenesis as well as permeabilization of blood vessels, and plays a central role in the regulation of vasculogenesis. Deregulated VEGF expression contributes to the development of solid tumors by promoting tumor angiogenesis and to the etiology of several additional diseases that are characterized by abnormal angiogenesis. Consequently, inhibition of VEGF signaling abrogates the development of a wide variety of tumors.

A VEGF inhibitor is any molecule, such as a DNA, RNA, oligonucleotide, protein, polynucleotide, peptide, or small molecule that blocks or diminishes the activity of VEGF compared to activity of VEGF in the absence of the molecule. Any assay known to those of ordinary skill in the art can be used to assess VEGF activity, and to determine activity of VEGF in the presence and absence of a molecule. Examples of VEGF inhibitors are discussed at length elsewhere in this specification.

The dose of VEGF inhibitor can be any dose known to those of ordinary skill in the art. For example, if the VEGF inhibitor is bevacizumab, the dose may be about 0.1 to about 10 mg/kg, or greater, by iv infusion. The dose can be tailored depending on side effects and clinical criteria, such as response to therapy. Bevacizumab should be permanently discontinued in patients who develop gastrointestinal perforation, wound dehiscence requiring medical intervention, serious bleeding, nephrotic syndrome, or hypertensive crisis.

### M. IL-10 Inhibitors

Interleukin-10 (IL-10) is a recently described natural endogenous immunosuppressive cytokine, identified in both the murine and human organism. Murine interleukin-10 (mIL-10) was originally described as a cytokine synthesis inhibitory factor released from T helper T-cell clones, but it also carries proliferative effects upon various subsets of lymphocytes, including an enhancing effect upon cloning efficacy of CD4−, 8+ murine splenic T cells. Human interleukin 10 (hIL-10) has recently been sequenced and revealed to have high homology with mIL10 at DNA sequence level as well as on amino acid level. Furthermore, swine interleukin 10 has recently been sequenced and revealed to have high homology with human IL-10 at DNA sequence level as well as on amino acid level. Also, hIL-10 has high homology with an open reading frame in the Epstein-Barr virus genome, BCRF1, and viral IL-10 does show some activity similar to hIL-10.

Human IL-10 is produced by activated T cell clones and immortalized B cells, and in addition to its cytokine synthesis inhibitory factor (CSIF) activity, inhibiting the production of several pro-inflammatory cytokines and colony-stimulating factors, it also induces the production of a natural interleukin-1 receptor antagonist protein/peptide (IRAP) by mononuclear cells, thereby indirectly inhibiting IL-1 activity. IL-10 also downregulates its own production by monocytes and inhibits the expression of class II MHC expression.

An IL-10 inhibitor is any molecule, such as a DNA, RNA, oligonucleotide, protein, polynucleotide, peptide, or small molecule that blocks or diminishes the activity of IL-10 compared to activity of IL-10 in the absence of the molecule. Information regarding specific IL-10 inhibitors has been set forth elsewhere in this specification. Any assay known to those of ordinary skill in the art can be used to assess IL-10 activity, and to determine activity of IL-10 in the presence and absence of a molecule.

The dose of IL-10 inhibitor can be any dose known to those of ordinary skill in the art. For example, the dose may be about 0.1 to about 10 mg/kg, or greater, by iv infusion. The dose can be tailored depending on side effects and clinical criteria, such as response to therapy.

### N. TNF

TNF is a member of a group of other cytokines that all stimulate the acute phase reaction. There are various members in this family, such as TNF-alpha and TNF-beta. TNF-alpha is a 185 amino acid glycoprotein peptide hormone, cleaved from a 212 amino acid-long propeptide on the surface of macrophages. Some cells secrete shorter or longer isoforms. TNFα is released by white blood cells, endothelium and several other tissues in the course of damage, e.g. by infection. Its release is stimulated by several other mediators, such as interleukin 1 and bacterial endotoxin. It has a number of actions on various organ systems, generally together with interleukins 1 and 6.

The dose of TNF can be any dose known to those of ordinary skill in the art. For example, the dose may be about 0.1 to about 10 mg/kg, or greater, by iv infusion. The dose can be tailored depending on side effects and clinical criteria, such as response to therapy.

### O. Taxotere

Docetaxel (Taxotere, made by Aventis) is an antineoplastic chemotherapeutic agent. Taxotere is indicated for the treatment of patients with locally advanced or metastatic breast cancer after failure of prior chemotherapy. The dose of taxotere can be any dose known to those of ordinary skill in the art. For example, the dose may be from about 1 mg/m² to about 1,500 mg/m or greater.

### P. Pharmaceutical Formulations and Delivery

In certain embodiments of the present invention, methods involving delivery of an expression construct encoding a MDA-7 protein are contemplated. In some embodiments, the method is directed to delivery of an expression construct encoding an immunogen. Alternatively, the expression construct comprises sequence encoding both the MDA-7 polypeptide and the immunogen. Examples of diseases and conditions involving an immune response include diseases that are prevented or treated with a vaccine. Including lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, breast cancer, bladder cancer and any other diseases or condition related to an immune response that may be treated by administering a MDA-7 polyprotein to enhance an induced immune response.

1. Effective Amount

An “effective amount” of the pharmaceutical composition, generally, is defined as that amount sufficient to detectably and repeatedly to achieve the stated desired result, for example, to ameliorate, reduce, minimize or limit the extent of the disease or its symptoms. More rigorous definitions may apply, including elimination, eradication or cure of disease.

2. Administration

In certain specific embodiments, it is desired to kill cells, inhibit cell growth, inhibit metastasis, decrease tumor or tissue size and otherwise reverse or reduce the malignant phenotype of tumor cells, induce an immune response, or inhibit angiogenesis using the methods and compositions of the present invention. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation.

Direct injection, intratumoral injection, or injection into the tumor vasculature is specifically contemplated for discrete, solid, accessible tumors or other accessible target areas. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as single dose comprise about 0.1 to about 0.5 ml volumes. The viral particles may advantageously be contacted by administering multiple injections to the tumor or targeted site, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising MDA-7 or an MDA-7-encoding construct together with or in the absence of an immunogenic molecule. The perfusion may be continued post-resection, for example, by leaving a catheter implanted at the site of the surgery. Periodic post-surgical treatment also is envisioned.

Continuous perfusion of an expression construct or a viral construct also is contemplated. The amount of construct or peptide delivered in continuous perfusion can be determined by the amount of uptake that is desirable.

Continuous administration also may be applied where appropriate, for example, where a tumor or other undesired affected area is excised and the tumor bed or targeted site is treated to eliminate residual, microscopic disease. Delivery via syringe or catherization is some. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, immune condition, target site, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor or affected area being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor or targeted site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher. Alternatively, the amount specified may be the amount administered as the average daily, average weekly, or average monthly dose.

Protein may be administered to a patient in doses of about or of at least about 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml, or any range derivable therein. Alternatively, any amount specified herein may be the amount administered as the average daily, average weekly, or average monthly dose.

COX-2 inhibitors can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

Concentrations of GA or its analogs and derivatives can be in doses of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more mg or mg/m² (with respect to tumor size or patient surface area), or any range derivable therein. Alternatively, the amount specified may be the amount administered as the average daily, average weekly, or average monthly dose, or it may be expressed in terms of mg/kg, where kg refers to the weight of the patient and the mg is specified above.

Concentrations of a vitamin E compound or its analogs can be in doses of about, at least about, or at most about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more mg, μg/ml, mg/kg (with respect to patient weight) or mg/m² (with respect to tumor size or patient surface area), or any range derivable therein. Alternatively, the amount of the vitamin E compound or analog may be expressed in terms of I.U. (international units). In certain embodiments, the amount of the vitamin E compound is about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more I.U, or any range derivable therein.

3. Injectable Compositions and Formulations

In some embodiments, the method for the delivery of an immunogenic molecule, an expression construct encoding a MDA-7 protein, MDA-7 protein, and/or an immunogen is via systemic administration. However, the pharmaceutical compositions disclosed herein may alternatively be administered parenterally, subcutaneously, directly, intratracheally, intravenously, intradermally, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising an MDA-7 (or encoding nucleic acid) and/or an MDA-7 conjunctive agent is in a water-based formulation. In other embodiments, the formulation is lipid based. It is specifically contemplated that the vitamin E compound may be in a a water- or lipid-based formulation.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Compounds and agents may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The MDA-7 protein (or fragments thereof) or a nucleic acid encoding all or part of MDA-7 may be formulated as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In certain formulations, an MDA-7 conjunctive agent is formulated as a dry power. It is a further object of the present invention to use, for the process, readily accessible cheap raw materials in the form of dairy by-products, in place of pure carbohydrates. It has been found that these objects can be achieved by a process for the preparation of a vitamin E dry powder containing a protein colloid and a disaccharide, wherein a vitamin E ester is dispersed in a residual liquor, low in alkaline earth metal ions and rich in lactose, from the production of lactose, in the presence of from 2 to 30% by weight, based on the solids content of the residual liquor, of a caseinate, and the dispersion is spray-dried, as set forth in U.S. Pat. No. 4,262,017, which is hereby incorporated by reference. The process gives a free-flowing vitamin E dry powder which has a pleasant flavor and may be used as an additive for foodstuffs and animal feeds. The dry powder furthermore has good tableting characteristics. Suitable vitamin E esters are the conventional esters of d- and d,1-α-tocopherol. Specific examples are vitamin E acetate, vitamin E succinate, vitamin E palmitate and vitamin E nicotinate. Amongst these, the acetate is preferred.

The protein, nucleic acid, or COX-2 inhibitor(s), Hsp90 inhibitors, or vitamin E compounds are administered in a manner compatible with the dosage formulation, and in such amount as will be therapeutically effective. The quantity to be administered depends on the subject to be treated, including, e.g., the aggressiveness of the cancer, the size of any tumor(s), the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

The manner of application may be varied widely. Any of the conventional methods for administration are applicable. These are believed to include oral application on a solid physiologically acceptable base or in a physiologically acceptable dispersion, parenterally, by injection or the like. The dosage will depend on the route of administration and will vary according to the size of the host.

In many instances, it will be desirable to have multiple administrations of each of both of the therapeutic agents (MDA-7 and COX-2 inhibitor, Hsp90 inhibitor, or Vitamin E compound).

### Q. Combination Treatments

In certain embodiments, the compositions and methods of the present invention involve an MDA-7 polypeptide, or expression construct coding therefor, and either a COX-2 inhibitor or an Hsp90 inhibitor, in combination with other agents including MDA-7 conjunctive agents or compositions to enhance the effect of MDA-7 or to increase any therapeutic, diagnostic, or prognostic effect for which the MDA-7 is being employed. These compositions would be provided in a combined amount effective to achieve the desired effect, for example, the killing of a cancer cell and/or the inhibition of angiogenesis. This process may involve contacting the cells with the expression construct and the agent(s) or multiple factor(s) at the same time. This may be achieved by contacting the cell with a single composition or pharmacological formulation that includes both or all agents, or by contacting the cell with two or more distinct compositions or formulations, at the same time, wherein one composition provides 1) MDA-7 (either as a protein or nucleic acid); and/or 2) either the COX-2 inhibitor(s) or the Hsp90 inhibitor (or other MDA-7 conjunctive agent); and/or 3) the third agent(s).

In embodiments of the present invention, it is contemplated that an mda-7 gene (or cDNA) or protein therapy is used in conjunction with a COX-2 inhibitor (referred to as “MDA-7/COX-2 inhibitor therapy”), in addition to a second or other anti-cancer agents or therapies. In other embodiments, it is contemplated that an mda-7 gene (or cDNA) or protein therapy is used in conjunction with an Hsp90 inhibitor (referred to as “MDA-7/Hsp90 inhibitor therapy”), in addition to a second or other anti-cancer agents or therapies. Alternatively, the MDA-7/COX-2 inhibitor therapy or MDA-7/Hsp90 inhibitor therapy may precede or follow the other anti-cancer treatment by intervals ranging from minutes to weeks. In embodiments where the MDA gene or protein therapy is provided to the patient separately from the COX-2 inhibitor or Hsp90 inhibitor, one would generally ensure that a significant period of time did not expire between the time of each delivery, such that the two compounds would still be able to exert an advantageously combined effect on the patient; alternatively, in embodiments where the MDA-7/COX-2 inhibitor therapy or MDA-7/Hsp90 inhibitor therapy is provided to the patient separately from the second anti-cancer therapy, one would generally ensure that a significant period of time did not expire between the time of each therapy, such that the two therapies would still be able to exert an advantageously combined effect on the patient. In such instances, it is contemplated that one may provide a patient with either 1) the MDA-7/COX-2 inhibitor therapy or 2) the MDA-7/Hsp90 inhibitor therapy and the second anti-cancer therapy within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. Instead of a COX-3 inhibitor or Hsp-90 inhibitor, the invention may be implemented in the context of another MDA-7 conjunctive agent.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example MDA gene or protein therapy is “A” and the MDA-7 conjunctive agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Alternatively, “A” could be an administration of the MDA-7/conjunctive agent therapy and “B” the administration of a second anti-cancer therapy. In other embodiments, MDA-7 gene or protein therapy is “A” and the MDA-7 conjunctive agent is “B”; or, “A” could be an administration of the MDA-7/MDA-7 conjunctive agent therapy and “B” the administration of a second anti-cancer therapy.

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to MDA-7 and/or an MDA-7 conjunctive agent. It is expected that the treatment cycles would be repeated as necessary. It also is contemplated that various standard therapies, as well as surgical intervention, may be applied in combination with the described therapy.

In specific embodiments, it is contemplated that a second anti-cancer therapy, such as chemotherapy, radiotherapy, immunotherapy or other gene therapy, is employed in combination, for example, with the MDA-7/COX-2 inhibitor therapy or the MDA-7/Hsp90 inhibitor therapy or any other MDA-7 conjunctive therapy, as described herein.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with MDA-7/COX-2 inhibitor therapy or MDA-7/Hsp90 inhibitor therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000).

Moreover, antibodies against any of these compounds can be used to target the anti-cancer agents discussed herein.

As discussed earlier, examples of immunotherapies currently under investigation or in use are immune adjuvants e.g., Mycobacterium bovis, Plasmodium falciparum, dinitrochlorobenzene and aromatic compounds (U.S. Pat. No. 5,801,005; U.S. Pat. No. 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. No. 5,830,880 and U.S. Pat. No. 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). It is contemplated that one or more anti-cancer therapies may be employed with the MDA-7 therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

4. Gene Therapy

In yet another embodiment, a combination treatment involves gene therapy in which a therapeutic polynucleotide is administered before, after, or at the same time as an MDA-7 polypeptide or nucleic acid encoding the polypeptide. Delivery of an MDA-7 polypptide or encoding nucleic acid in conjunction with a vector encoding another gene products may have a combined therapeutic effect on target tissues.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Hormonal Therapy

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### R. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synergistic Tumoricidal Effect with Celecoxib and AD-MDA7

A. Materials and Methods

1. Cell Lines

Estrogen receptor positive MCF7 cells engineered to express elevated levels of HER-2/neu (MCF7/Her18 cells) were a gift from Dr. Mien-Chie Hung. The estrogen receptor negative and HER-2/neu-nonoverexpressing MDA-MB-436 human breast cancer cells were obtained from the American Type Culture Collection (ATCC, Manassas, Va.). The cells were maintained in high glucose DMEM/F-12 media supplemented with 10% fetal bovine serum with 10 mM L-glutamine, 100 U/ml penicillin, and 100 μg/ml streptomycin (GIBCO Invitrogen Corporation, Grand island, NY) in a humidified 37° C., 5% CO₂ atmosphere.

2. Adenovirus Transduction & Celecoxib Treatment

The recombinant adenovirus vectors carrying the mda-7 gene (Ad-mda7) and the luciferase reporter gene (Ad-luc) were obtained from Introgen Therapeutics (Introgen Therapeutics, Houston, Tex.). 1×10⁶ cells in 100-mm culture plates were transduced with Ad-mda7 or Ad-luc at an MOI of 2000 or 1000 viral particles (vp) per cell (100 or 50 plaque-forming units/cell) for MCF7/Her18 and MDA-MB-436 cell lines, respectively. Celecoxib was dissolved in DMSO and added to cell culture media at a final concentration less than 0.1% (in order not to affect cell survival)—and then introduced into the cultures at a dose of 20 or 50 μM for MCF7/Her18 and MDA-MB-436 cells, respectively. The doses of vector and celecoxib were selected to ensure toxicity of less than 50% in order to compare the combinatorial effect of Ad-mda7 and celecoxib.

3. Cell Proliferation Assay

The effect of celecoxib and Ad-mda7 on human breast cancer cell growth was determined by cell counting after trypan blue (Invitrogen Co., Carlsbad, Calif.) exclusion and MTT (3-[4,5-dimethylthiazol-2-yl]-2,5-diphenyl tetrazolium bromide) (Sigma, St. Louis, Mo.) assays. Briefly, cells were seeded at a density of 6×10⁵ cells per 60-mm culture plate. After 24 hours, the media was removed, and culture media with or without celecoxib was added as planned, and viral transduction was performed, as described. 15 After a 72-hour incubation, floating and adherent cells were harvested and the combined cell populations were counted using a hemocytometer. Cell viability was determined by trypan blue exclusion staining. For MTT assays, 1,000 cells were seeded in triplicates in 96-well plates and assayed 72 hours later. After incubation, cells were fixed with DMSO, and stained with MTT solution (5 mg/ml). The absorbance was read with an automated spectrophotometric miniplate reader (EL808 Ultramicroplate reader, Bio-Tek Instruments, Inc., Winooski, Vt.) at 570 nm. Values were normalized and plotted as the percentage change compared to control cells (means±S.E.M.).

4. Cell Cycle and Apoptosis Assay

All the cultures were subconfluent at the time of harvest. Harvested cells were fixed with ice-cold 80% ethanol, stained with propidium iodide (PI) (Sigma, St. Louis, Mo.), and analyzed using a flow cytometer (FCM) (EPICS XL-MCL, Coulter, Miami, Fla.) as described previously. Cells floating in the media and trypsinized adherent cells were harvested and pelleted. The collected cells were washed, and then stained with Annexin V-fluorescein isothiocyanate (FITC)/PI using an Annexin V/FITC apoptosis detection kit (BD Biosciences, Franklin Lake, N.J.). The BrdU (5-bromo-2-deoxyuridine)/terminal deoxynucleotidyl transferase (TdT)-mediated 2′-deoxyuridine 5′-triphosphate (dUTP)-biotin nick end labeling (TUNEL) assay (APO-Direct, BD Biosciences, Franklin Lake, N.J.) was performed according to the manufacturer's protocol. Briefly, paraformaldehyde-fixed cells were washed and incubated with staining solution (10 μl of TdT reaction buffer, 0.75 μl of TdT enzyme, and 8 μl of FITC-dUTP) overnight. The following day, cells were rinsed and resuspended in 1 ml of PI/RNase solution. Following incubation in the dark for 30 minutes at room temperature, flow cytometry was performed to obtain the percentage of apoptotic cells. Cell cycles were analyzed with a program combined with the FCM (Multicycle, Phoenix Flow System, San Diego, Calif.).

5. Prostaglandin E₂ (PGE₂) Measurement

In order to determine the concentration of PGE₂, cells were seeded at a density of 1×10⁶ cells/100-mm plates, and treated with celecoxib, Ad-mda7, or a combination of both agents. After a 72-hr incubation, 3 μl of arachidonic acid (1 mM) was added to the culture media to boost the PGE₂ production for 30 minutes. The supernatant was collected and stored at −80° C. until PGE₂ concentration was measured using an enzyme-linked immunoassay kit (Cayman Chemical, Ann Arbor, Mich.) according to the manufacturer's manual. The final results from the triplicated values were expressed as pg/ml.

6. Western Blotting

Cells were lysed and protein concentration determined using the BioRad Assay (Bio-Rad Laboratories, Hercules, Calif.). Lysates were analyzed by western blot analysis using 10% SDS gels. Lanes were loaded with 50 ug of protein and electrophoresed for 2 hrs at 90 V. Gels were transferred to nitrocellulose membranes that were blocked with 5% nonfat dry milk and incubated with primary antibodies (COX-2 (Cayman Chemical Co., Ann Arbor, Mich.), β-catenin (Santa Cruz Biotechnology, Inc., Santa Cruz, Calif.), Akt (Cell Signaling, Beverly, Mass.) and p-Akt (Cell Signaling, Beverly, Mass.)) overnight at 4° C. Membranes were washed and incubated with secondary antibody for 1 hr at room temperature. Membranes were then developed and protein signals detected using enhanced chemiluminescence (ECL) western blotting detection reagents (Amersham Biosciences, Buckinghamshire, UK). Membranes were incubated with antibody against β-actin (Santa Cruz Biotechnology, Santa Cruz, Calif.) to assess equal protein loading. Results were subjected to densitometry.

7. Statistical Analysis

Statistical analysis was performed between control and treated groups, and among the different experimental groups. Comparisons of means were carried out using the Student's t test. The densitometry of the western blots were also analyzed for significance by the Student's t test. Differences with a value of p<0.05 were considered to be statistically significant.

B. Results

1. Ad-mda7 and Celecoxib Cotreatment Inhibit Growth of Breast Cancer Cells

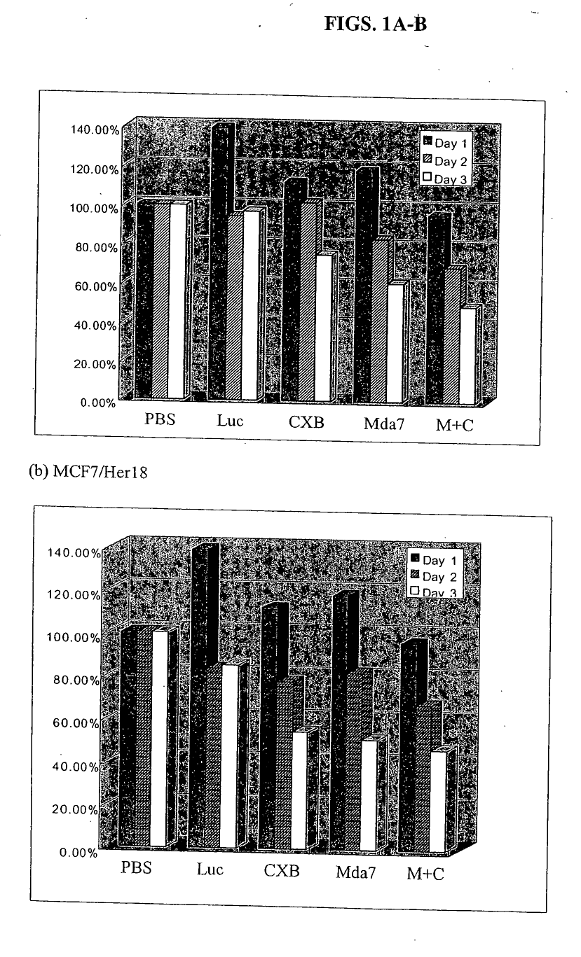

After being treated with Ad-mda7 and/or celecoxib, cell viability was assessed using trypan blue exclusion and MTT assays. As shown in FIG. 1 using MTT assays, the combination treatment group showed significantly decreased cell viability after 48- and 72-hour incubation, compared to the control, regardless of the expression status of HER-2/neu. HER-2/neu (+) cells showed differences in the number of viable cells between Ad-mda7 and combined treatment after 24-hr treatment (p=0.04), significant decreases in viability in the combination group compared to controls after 48-hr treatment (p=0.045), and all treatment groups showed statistically significant decreases in survival compared to controls (p=0.002 for celecoxib, p=0.02 for Ad-mda7, and p=0.009 for the combination). HER-2/neu (−) cells showed no differences among groups after 24 hours, but the combination started to show differences compared to controls (p=0.049). Combination treatment and Ad-mda7 treatment appeared to be more effective in killing the HER2/neu (−) cells than celecoxib (p=0.03 and p=0.02, respectively), and the combination was superior to Ad-mda7 in tumor cell killing (p=0.02) on day 2.

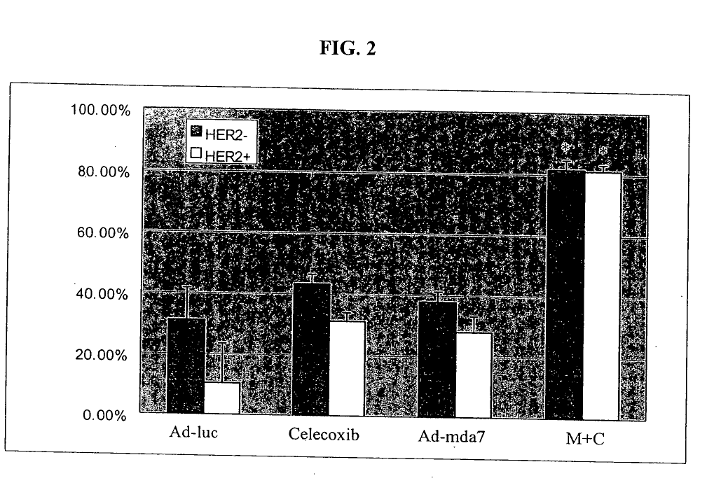

After 72-hr treatment, Ad-mda7 was more effective than controls (p=0.02), and the combination was superior to celecoxib in cytotoxicity (p=0.01). As shown in FIG. 2 using trypan blue exclusion, celecoxib and the combination treatment showed greater efficiency in tumor cell killing numbers compared to controls in HER-2/neu (+) cells (p=0.04 and p=0.01, respectively). Between the three treatment groups in MCF7/Her18 cells, neither celecoxib nor Ad-mda7 demonstrated increased cytotoxicity compared to the combination (p=0.01). In MDA-MB-436 cells, the combined treatment was the most effective treatment arm compared to control, Ad-mda7, or celecoxib (p=0.01, 0.01 and 0.01, respectively). The effect of combination treatment on PGE₂ production was also determined in those cells (Table 4). Reproducibly, the combination showed greater inhibition of PGE₂ production compared to the controls (p=0.01 for HER-2/neu (+) and p=0.049 for HER-2/neu (−) cells). Treatment with monotherapy Ad-mda7 in the MDA-MB-436 cells failed to show a significant decrease in the amount of PGE₂ produced (p=0.06), in contrast to the marked decrease found in MCF7/Her18 cells (p=0.04). Celecoxib reduced PGE₂ production in both cell lines (p=0.03 for MCF7/Her18 and p=0.049 for MDA-MB-436).

TABLE 4
Prostaglandin E₂ production (pg/ml) in breast cancer cells after 72-hour
treatment with monotherapy or combination therapy.

| | MCF7/Her18 | MDA-MB-436 |
|---|---|---|
| PBS | 85.1 | 5469.8 |
| Luciferase* | 75.1 | 5255.5 |
| Celecoxib† | 16.7^{§} | 116.7^{§} |
| Ad-mda7‡ | 67.9^{§} | 1064.2 |
| Ad-mda7 + Celecoxib | 10.9^{§} | 71.3^{§} |

*Multiplicity of infection (MOI) of 2000 vp/cell for MDA7/Her18 and 1000 vp/cell for MDA-MB-436 cells
†20 uM for MCF7/Her18, 50 uM for MDA-MB-436
‡Ad-mda7(recombinant adenovirus encoding for the melanoma-differentiation associated gene-7), MOI of 2000 vp/cell for MCF7/Her18 and 1000 vp/cell for MDA-MB-436
^{§}p < 0.05, compared to the controls

2. Ad-mda7 and Celecoxib Combination Increases Apoptosis Compared to Individual Treatments

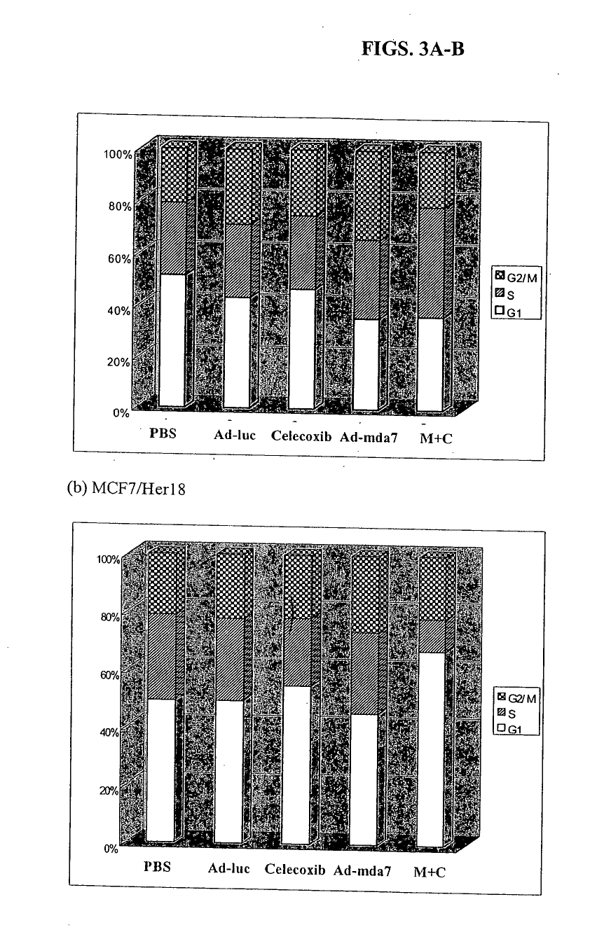

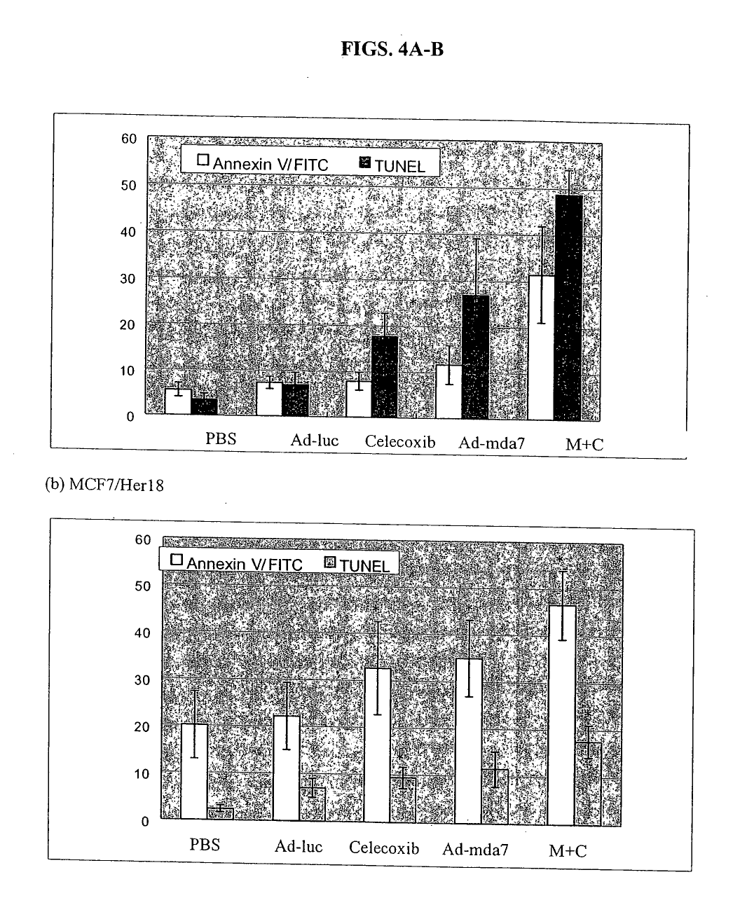

Previous studies using tumor cell lines have documented that Ad-mda7 induces G2/M cell cycle arrest whereas celecoxib induces a G1 block. The Ad-mda7 and celecoxib combination blocked MCF7/Her18 cells at the G1 phase of the cell cycle (p=0.03) and was significantly more pronounced than the G1 block mediated by celecoxib alone. In HER2/neu (−) cells, combination treatment resulted in an increase in cells in S-phase compared to celecoxib or Ad-mda7 (FIG. 3). In MCF7/Her18 and MDA-MB-436 cells, celecoxib blocked more cells at the G1 checkpoint than Ad-mda7 (p=0.02), whereas Ad-mda7 monotherapy resulted in a G2/M block (FIG. 3).

Early and late apoptotic events were evaluated using Annexin V-FITC and TUNEL assays (FIG. 4); both assays demonstrated significant increases in apoptosis induced by the combination treatment compared to monotherapy or controls. Furthermore, increased apoptosis was observed regardless of HER-2/neu expression status (p<0.05). Celecoxib and Ad-mda7 promoted apoptosis in both cell lines (p<0.05) by Annexin V/FITC assay. The increased Annexin V staining compared to TUNEL in HER-2/neu (+) cells may reflect the more rapid kinetics of apoptosis in MCF7/Her18 cells compared to MDA-MB-436.

3. Ad-mda7 and Celecoxib Combination Decreases Expression of COX-2, Akt and p-Akt

Ad-mda7 treatment is known to negatively regulate expression of Akt and p-Akt (Mhashilkar et al., 2003). Similar effects were observed for β-catenin expression after Ad-mda7 transduction. To elucidate the role of Ad-mda7 and celecoxib on the expression of representative prosurvival markers after the combination of Ad-mda7 and celecoxib, western blots were performed to analyze steady-state levels of COX-2, Akt, p-Akt, and β-catenin.

The level of Akt and p-Akt was determined by Western blotting analysis and densitometry. HER2-(MDA-MB-436) and HER2+(MCF7/Her18) cells showed significantly decreased expression of Akt and p-Akt after the combined treatment, compared to control (PBS). The relative densitometry numbers for Akt in the HER2+cells were celecoxib (1.01), Ad-mda7 (0.99) and both (0.53*) (*p<0.05). The relative densitometry numbers for pAkt in the HER2+ cells were celecoxib (0.61*), Ad-mda7 (1.85) and both (0.36*) (*p<0.05). The relative densitometry numbers for Akt in the HER2-cells were celecoxib (0.72), Ad-mda7 (1.00) and both (0.44*) (*p<0.05). The relative densitometry numbers for pAkt in the HER2-cells were celecoxib (0.67), Ad-mda7 (1.61) and both (0.37*) (*p<0.05).

The relative level of COX-2 was determined by Western blotting analysis and densitometry. HER2− (MDA-MB-436) and HER2+ (MCF7/Her18) cells showed significantly decreased expression of COX-2 after the combined treatment, compared to control (PBS). The relative densitometry numbers for COX-2 in the HER2+ cells were celecoxib (0.53), Ad-mda7 (0.78) and both (0.53*) (*p<0.05). The relative densitometry numbers for COX-2 in the HER2− cells were celecoxib (0.74), Ad-mda7 (0.78) and both (0.45*) (*p<0.05).

The relative level of β-catenin was determined by Western blotting analysis and densitometry. HER2− (MDA-MB-436) and HER2+ (MCF7/Her18) cells showed no significant differences in the expression of β-catenin. The relative densitometry numbers for β-catenin in the HER2+ cells were celecoxib (0.78), Ad-mda7 (0.64) and both (0.85). The relative densitometry numbers for β-catenin in the HER2− cells were celecoxib (0.82), Ad-mda7 (0.97) and both (0.61).

Significantly decreased levels of Akt, p-Akt, and COX-2 were noted following treatment, regardless of HER-2/neu expression (p<0.05). In addition to the combination treatment, celecoxib showed more potent ability to inhibit phosphorylation of Akt over controls in MCF7/Her18 cells (p=0.04). The expression of Akt is somewhat repressed by celecoxib compared to control in MDA-MB-436 cells (p=0.054). Ad-mda7 increased p-Akt in both cell lines, although the increase was also observed with Ad-luc, suggesting that the effect was not MDA-7 protein dependent. The combination of Ad-mda7 and celecoxib reduced p-Akt by >70% compared to control and by approximately 50% compared to celecoxib monotherapy. Both Ad-mda7 and celecoxib reduced COX-2 expression and further COX-2 inhibition was seen by the combination in MDA-MB-436 cells. Both Ad-mda7 and celecoxib reduced β-catenin in MCF7/Her18 cells and only slightly reduced β-catenin levels in MDA-MB-436 cells. The combination reduced β-catenin levels in both cell lines, however the down-regulation was not statistically significant.

C. Discussion

A unique property of Ad-mda7 is inhibitions of cancer cell growth and induction of apoptosis without affecting normal cells (Mhashilkar et al., 2003; Pataer et al., 2002; Jiang et al., 1996; Saeki et al., 2000). One mechanism of action in cancer cells that may be responsible for tumor cell killing is the down-regulation of the prosurvival mediators Akt and phosphorylated Akt (Mhashilkar et al., 2003; McKenzie et al., 2004). Cylooxygenase 2 (COX-2) is one of the enzymes required to metabolize arachidonic acid for the production of various kinds of prostaglandins. The other enzyme in the cascade, cyclooxygenase 1, is constitutively expressed in cells, but COX-2 is distinctive in that it is often induced or up-regulated in tumor cells. Recently, the enhanced expression of COX-2 in various kinds of human cancers has been recognized leading many investigators to examine the role of selective or nonspecific COX-2 inhibitors to prevent or treat cancers. Aspirin and other nonsteroidal anti-inflammatory drugs have demonstrated effectiveness in the use of chemoprevention in many cancers, especially colon cancer (Steinbach et al., 2000; Thun et al., 1991). More recently there has been an increasing number of reports examining the potential application of selective COX-2 inhibitors, including celecoxib, in preventing and treating various cancers including breast cancer (Basu et al., 2004; Liu et al., 2003; Howe et al., 2002).

Among the complex signal transduction, PI3K/Akt has received significant attention for its role in regulation of apoptosis and survival pathways (Mhashilkar et al., 2003. First isolated as a retroviral oncogene, PI3K drives prosurvival pathways in several human cancers (Fry, 2001). PKB/Akt, a serine-threonine protein kinase, regulated by the intracellular level of phospholipids, appears to play a key role in oncogenesis (Knuefermann et al., 2003). Because PKB/Akt, a serine-threonine protein kinase, is the downstream target of PI3K, it is amplified or activated by phosphorylation at Thr³⁰⁸ and Ser⁴⁷³ in various human cancers (Marte and Downward, 1997). The PI3K/Akt survival pathway has been shown to regulate the NF-kappaB (NF-κB) signaling pathway and to suppress the induction of tumor necrosis factor (TNF)-induced apoptosis (Burow et al., 2000). HER-2/neu promotes activation of the PI3K/Akt pathway which then activates NF-κB, leading to inhibition of apoptosis. In addition, the PI3K/Akt signaling pathway can provoke cancer cell migration mediated by transforming growth factor beta (TGF-β) (Bakin et al., 2000). Thus, recent investigations have focused on the regulation or inhibition of Akt activity in the treatment of various cancers. It was recently reported that celecoxib, one of the potent and selective COX-2 inhibitors, induced apoptosis in cancer cells in vitro through inhibition of Akt activation (Hsu et al., 2000). Adenoviral vectors have been widely used successfully to transfer therapeutic genes in vitro and in vivo.

It might have been predicted that the combination of Ad-mda7 and celecoxib would decrease phosphorylation of Akt, and the enhanced tumoricidal effect would be more noticeable in the HER-2/neu-overexpressing cells, due to the positive feedback loop between PGE₂ and HER-2/neu receptor expression (Benoit et al., 2004). In fact, these experiments successfully demonstrated enhanced anti-tumor activity by combining celecoxib and Ad-mda7 in both HER-2/neu-positive and HER-2/neu-negative breast cancer cells. This occurred through direct inhibition of COX-2 expression and through down-regulation of the PI3K/Akt prosurvival pathway. The use of Ad-mda7 and celecoxib in combination can provide several advantages, including enhancing apoptosis and inhibition of tumor cell growth at doses lower than that needed for either agent to be effective as a monotherapy.

### Example 2

### Radiosensitization with MDA7 and Celecoxib

A. Materials and Methods

MDA-MB-436 and MDA-MB-468 human breast cancer cells (see Example 1) were exposed to different doses of radiation with or without pretreatment with Ad-mda7 alone, celecoxib alone, or the combination of both for three days prior to irradiation. The cells were assayed for clonogenic survival to compare the radiosensitizing effect of three different treatment arms. Flow cytometry and cell cycle analysis were performed to access cell cycle changes and induction of apoptosis. Statistical evaluation was done by student's t-test.

B. Results

The clonogenic survival assay showed that the combination of Ad-mda7 and celecoxib significantly enhanced tumor cell radiosensitization in both breast cancer cell lines. At the sublethal dose, less than 50% tumoricidal effect of celecoxib (50 μM for MB436 and 30 μM for MB468) and Ad-mda7 (multiplicity of infection (MOI) of 1,000 for MB436 and 2,000 for MB468), the combination showed significantly enhanced radiosensitivity of both cell lines (p<0.05). There was an increased percentage of apoptotic cells in the combination therapy group as compared to the controls but this was not statistically significant. Cell cycle analysis demonstrated an increase in the G2/M cell cycle in the combination group compared to controls.

### Example 3

### Enhancement of AD-MDA7 Cell Killing with Geldanamycin and its Analog

A. Materials and Methods

1. Cell Lines and Reagents

A549 and H460 human lung cancer cell lines were obtained from the American Type Culture Collection. All cells were maintained in RPMI 1640 supplemented with 10% fetal bovine serum, 10 mM glutamine, 100 units/ml penicillin, 100 μg/ml streptomycin (Life Technologies, Inc., Grand Island, N.Y.) in a 5% CO₂ atmosphere at 37° C. Geldanamycin (GA) was obtained from Calbiochem (San Diego, Calif.). 17-allyl-aminogeldanamycin (17AAG) was kindly provided by Dr. Nguyen Dao (National Cancer Institute, Bethesda, Md.). 17AAG was formulated in DMSO (Sigma Chemical Co., St. Louis, Mo.) as 10-mM stock solutions, and stored at −20° C. Final working solutions were diluted in medium to contain <0.01% of DMSO. All experiments using this compound were performed under subdued lighting conditions.

2. Adenovirus Production

Constructions of the Ad-mda7, Ad-LacZ and Ad-Luc vectors have been previously reported (Pataer et al., 2002). The transduction efficiencies of adenoviral vectors in A549 and H460 cancer cell lines were determined by infecting cells with Ad-LacZ and then determining the titers needed to transduce at least 70% of the cells.

3. Flow Cytometry Analysis

Apoptosis of cells was measured by propidium iodide staining and FACS analysis. Cells were harvested, pelleted by centrifugation and resuspended in phosphate-buffered saline containing 50 μg/ml propidium iodide, 0.1% Triton X-100, and 0.1% sodium citrate and vortexed prior to FACS analysis (Becton-Dickenson FACScan, Mountain View, Calif.; FL-3 channel).

4. Western Blot Analysis

At 48 h after transfection, the cell extracts were prepared and immunoblot assays was performed as described before (Pataer et al., 2002). The following antibodies were used: PKR (K-17), HSP90, β-catenin, E-cadherin, Raf-1, and β-actin antibodies were bought from Santa Cruz Biotechnology (Santa Cruz, Calif.). Phospho-PKR [pT451] and Phospho-eIF-2a [pS51] antibodies were purchased from BioSource International (BioSource International, Camarillo, Calif.). Akt and phospho-Akt (Ser473) were bought from Cell Signaling (Cell Signaling; Beverly, Mass.). The polyclonal or monoclonal antibody to MDA-7 was obtained from Introgen Therapeutics Inc (Houston, Tex.).

5. Immunofluorescence Analysis

A549 cells (5×10⁴ cells/well) were grown on chamber slides until 70% confluence and then treated with Ad-luc, Ad-mda7, GA, Ad-mda7 plus GA or Ad-luc plus GA. Forty-eight hours later, cells were washed with PBS and fixed with freshly made 4% paraformaldehyde/PBS for 15 minutes. Cells were then permeabilized for 20 min at 4° C. with 0.2% Triton X-100 and blocked one hour with 1% normal goat serum. Rabbit polyclonal anti-beta-catenin were incubated overnight at 4° C. and developed with rhodamine secondary antibodies for 30 min at 37° C. Cells were then visualized under the fluorescence microscope (Olympus BX50 fluorescent microscope) (Vorburger et al., 2002).

6. Immunoprecipitation Analysis

Cells were treated with PBS, Ad-mda7, Ad-mda7 plus GA or GA alone for 48 hrs, and then lysed in RIPA buffer (1×PBS, 1% Nonidet P-40, 0.5% Sodium deoxycholate, 0.1% SDS). 500 μl (500 μg) cell lysate was incubated with primary antibody overnight at 4° C. Protein A/G agarose was added to the mix and incubated for 4 hrs. The beads were pelleted by centrifugation at 2500 rpm for 5 min at 4° C. The pellet was washed four times with 1 ml of RIPA buffer. After the last wash, 50 μl of 1×SDS-PAGE sample buffer was added to the beads, which were then vortexed, and boiled for 5 min. This was then centrifuged at 2500 rpm for 1 min before loading the supernatant on a gel.

7. Motility Assay

Medium (0.7 ml) was added to each well of a 24-well plate (Costar). Cell culture inserts (Fisher; 8 μm pore size, Falcon 3097) were placed into each well. A549 and H460 cells were adjusted to a concentration of 5×10⁵ cells/ml, and 500 μl of cells were placed into each insert. Cells were incubated for 36 h with PBS, Ad-luc, Ad-mda7, GA, Ad-mda7 plus GA and Ad-luc plus GA. After 36 h, the number of cells adherent to the bottom of the well was counted. Motility is expressed as a percentage of the number of cells in drug-free wells adhering to the well after 36 h.

8. Statistical Analysis

The data reported represents the mean of three or more independent experiments and the bars show the standard deviation (SD). ANOVA and two-tailed Student's t test were used for statistical analysis of multiple groups and pair-wise comparison, respectively, with P<0.05 considered significant.

B. Results

1. Geldanamycin (GA) Enhance Adenoviral mda-7 Mediated Cell Killing in Human Lung Cancer Cells.

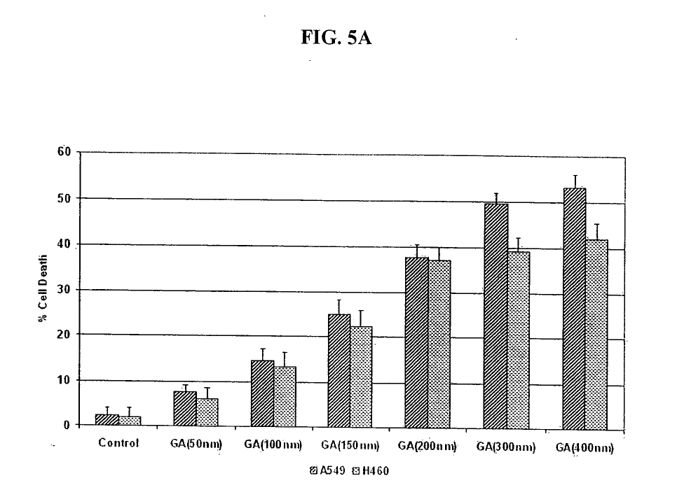

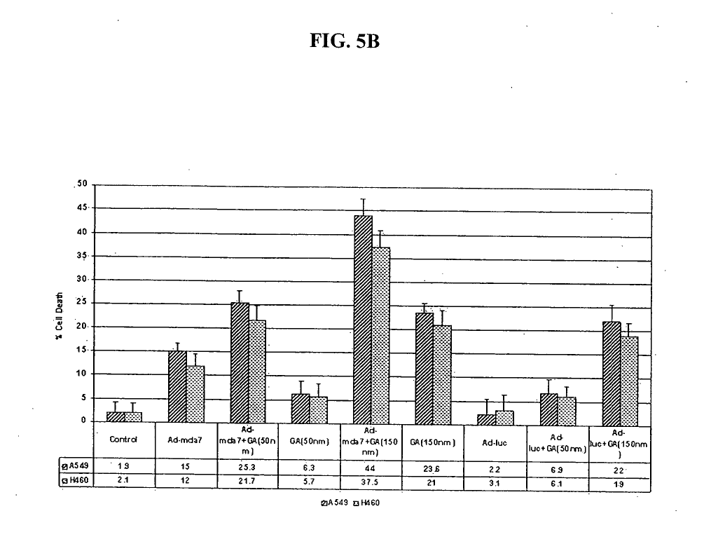

Many investigators have shown that Geldanamycin (GA) could induce cell death on breast and colon cancer cells. Whether GA could induce apoptosis in human lung cancer A549 and H460 cells was investigated. Flow cytometric analysis was performed in the A549 and H460 cell lines 48 h following exposure with different does of GA. FIG. 5A shows that treatment of lung cancer cells with GA resulted in high percentage of cell death in both cell lines. Cell death was dose-dependent induced by GA in these cancer cell lines. The effects of Ad-mda7 combined with 50 nm and 150 nm does of GA for 48 h were examined in both A549 and H460 cell lines. Flow cytometric analysis showed that Ad-mda-7 alone resulted in 15 and 12 percentages of apoptosis in A549 and H460 cells respectively. GA alone at 50 nm resulted in 6.3 and 5.7 percentages of apoptosis in A549 and H460 cells respectively at 48 hrs. GA alone at 150 nm resulted in 23.6 and 21 percentages of apoptosis in A549 and H460 cells respectively at 48 hrs. GA and Ad-mda7 combination resulted in a substantial enhancement of apoptotic cells in both A549 (25.3 and 44%) and H460 (21.7 and 37.5%) cells (FIG. 5B). This enhancement of apoptotic effect does not appear with the GA and Ad-luc combination in these cancer cells (FIG. 5B). Moreover, the combined effect was greater than the added effects of each agent.

2. Ad-mda7 and GA Combination Treatment does not Increase the Expression of PKR

The inventors had previously reported that Ad-mda7 induces and activates the ds-RNA dependent protein kinase (PKR), which leads to phosphorylation of eIF-2alpha and the induction of apoptosis in lung cancer cells. It was therefore tested if the Ad-mda7 and GA combination treatment increased the expression levels and phosphorylation status of the PKR. A549 and H460 cells treated with Ad-mda7 displayed increases in the amounds of PKR and phosphorylated PKR. In contrast, PBS, GA or Ad-luc treatment did not result in an increase in PKR or in phosphorylation of PKR. The combination of Ad-mda7 with GA did not increase PKR levels or its phosphorylation status in both A549 and H460 cells. In contrast, treatment of these cancer cells with GA caused degradation of AKT, P-AKT, Raf targets, which require Hsp90 for conformational maturation. Interestingly, Ad-mda7 degradated AKT, but at same time increased phosphorylation status of AKT in both A549 and H460 cells. Ad-mda7 and GA cotreatment significantly degraded phosphorylated AKT in these cancer cells. These results suggest that inhibition of Ad-mda7 mediated activation of AKT may in part be attributable to the synergistic effect of Ad-mda7 and GA.

3. Ad-mda7 and GA Combination Up-Regulate Surface E-Cadherin and Increasing of β-Catenin/E-Cadherin Association in Lung Cancer Cells

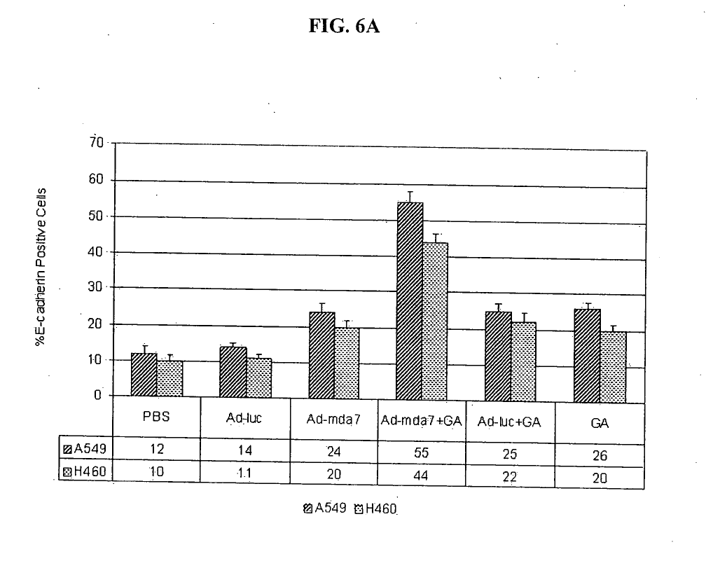

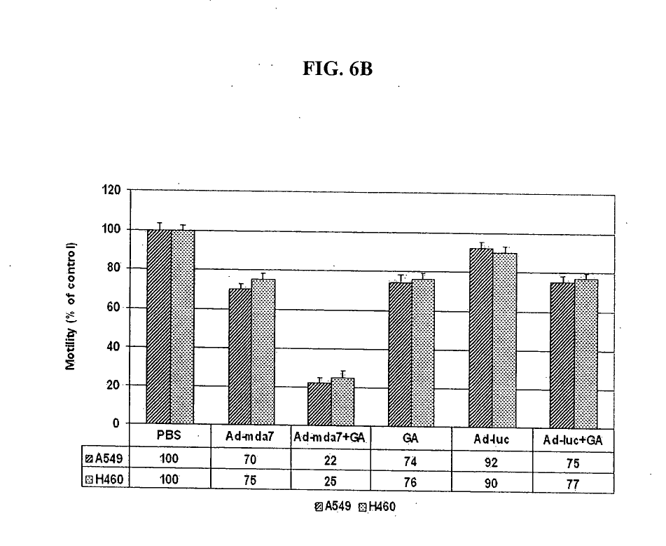

Previous reports had shown that Ad-mda7 could up-regulate E-cadherin in human lung cancer cells (Mhashilkar et al., 2003). The inventors investigated whether the combination of Ad-mda7 and GA could enhance up-regulation of E-cadherin in human lung cancer cells. As show in FIG. 6A, Ad-mda7 alone and GA alone could each increase in E-cadherin levels in A549 and H460 cells, as determined by surface staining using anti-E-cadherin monoclonal antibody and flow cytometry. Compared with PBS, Ad-luc, Ad-mda7, GA alone or Ad-luc plus GA treatment, the Ad-mda7 and GA (50 nm) combination treatment further increased the level of E-cadherin in these cells. Immunofluorescence staining showed that Ad-mda7 alone and GA alone could each increase in β-catenin levels in A549 cells. The staining experiments also showed that the Ad-mda7 and GA combination markedly increased β-catenin levels in these cells.

The previous study also had demonstrated that geldanamycin can stimulate tyrosine dephosphorylation of β-catenin and increased β-catenin/E-cadherin association, resulting in substantially decreased cell motility (Bonvini et al., 2001). It was therefore, investigated whether the combination of Ad-mda7 and GA could increase β-catenin/E-cadherin association. PBS-, Ad-mda7-, GA- or combination-treated cells were first immunoprecipitated with anti-E-cadherin antibody and then immunoblotted with a β-catenin-specific antibody. This showed that the amount of β-catenin coimmunoprecipitated with E-cadherin dramatically increased in cells treated with the combination of MDA-7 and GA, compared to the levels seens in both A549 and H460 cells treated with PBS, Ad-mda7 or GA alone. The same level of E-cadherin also could be detected when the immunoprecipitates were immumoblotted with anti-E-cadherin antibody in both cell lines.

Because the abundance of membrane-associated β-catenin-E-cadherin complexes is inversely related to cell motility, whether Ad-mda7 and GA combination could reduce the motility of A549 and H460 cells was investigated in vitro. When added to a 36-h in vitro motility assay, Ad-mda7 or GA (50 nM) alone reduced motility in both cell lines without affecting cell viability as assessed by trypan blue staining (FIG. 6B). Ad-mda7 and GA co-treatment resulted in substantially decreased cell motility on both A549 and H460 cells (FIG. 6B). This result did not appear when co-treated with Ad-luc and GA in both cell lines (FIG. 6B).

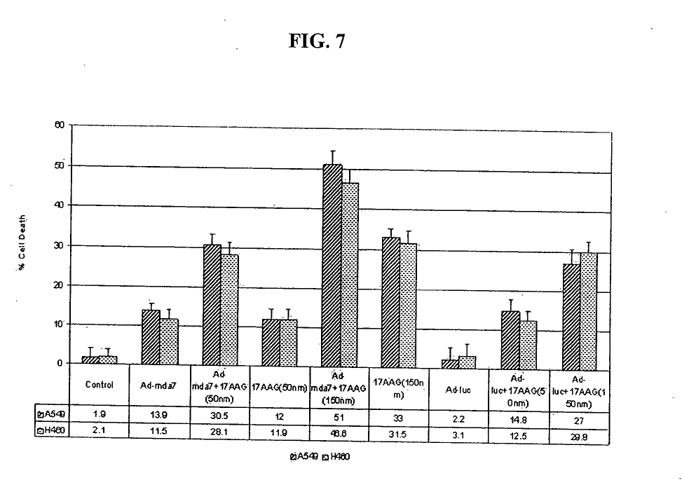

It was next investigated whether 17AAG—a GA analog—could have the same effect as GA on human lung cancer cells treated with MDA-7. Flow cytometric analysis was performed on the A549 and H460 cell lines 48 h following exposure with two different doses of 17AAG. FIG. 7 showed that treatment of lung cancer cells with 17AAG or Ad-mda7 alone resulted in cell death with both cell lines. Compared with combination of Ad-luc and 17AAG, the combination of 17AAG and Ad-mda7 resulted in a significant enhancement of apoptosis in both A549 and H460 cells (FIG. 7).

### Example 4

### Enhancement of AD-MDA7 Growth Inhibition Effect in Combination with Vitamin E Succinate (VES)

A. Materials and Methods

1. Cell Lines and Reagents

Human ovarian cancer cells MDAH 2774 were obtained from Dr. Judith Wolf at MD Anderson Cancer Center. The normal fibroblast cell line MRC-9 were obtained from ATCC. The Ad-luciferase and Ad-MDA7 vectors were obtained from Introgen Therapeutics (see for example, Mhashilkar et al., 2001, which is incorporated herein by reference). Vitamin E succinate was obtained from Sigma Chemicals, St. Louis, Mo.

2. Assay for Growth Inhibition

Human ovarian cancer cells (MDAH 2774) or normal human fibroblast cells (MRC-9) were treated with Ad-luc (vector control), Tocopherol (Vitamin E succinate, 8 μg/mL), Ad-mda7 (2000 vp/cell) or a combination thereof for 72 h (3 days).

Cells were infected with Ad-luc or Ad-mda7 for 3 h in serum free medium. After 3 h of incubation cells were replenished with complete medium. At this time tocopherol dissolved in DMSO was added to the cells to give a final concentration of 8 μg/ml. After 72 h of incubation, cells were harvested and the percent growth inhibition was determined by tyrpan exclusion method.

3. Western Blot Analysis

For Western blot analysis, total protein was isolated from MDAH 2774 cells treated with Ad-luc (vector control), Tocopherol (Vitamin E succinate, 8 μg/mL), Ad-mda7 (2000 vp/cell) or a combination thereof by adding cell lysis buffer (20 mM HEPES, pH 7.5; 10 mM KCl, 1 mM MgCl2, 1 mM EDTA, 1 mM DTT, 250 mM sucrose and 1× protease inhibitor). Proteins were separated by SDS Polyacrylamide Gel Electrophoresis and immobilized on nylon membrane. Membranes were probed with primary antibodies against Caspase-9 (Cell Signaling, Boston, Mass.), and Caspase-3, Poly(ADP-ribose) polymerase PARP, Bid, and Caspase-8 (BD-Pharmingen, San Diego, Calif.); Fas and MDA-7 (Introgen Therapeutics); cytochrome C (Santa Cruz Biotechnology, Santa Cruz, Calif.); and, β-actin. Protein expression was determined by using the appropriate horseradish peroxidase (HRP)-conjugated secondary antibodies and visualized on enhanced chemiluminescence film (Hyperfilm, Amersham) by application of Amersham's enhanced chemiluminescence western blotting detection system.

4. Cell Fractionation

Cells were fractionated into cytoplasmic and mitochondrial fraction for detection of cytochrome C release as previously described (Gewies et al., Cancer Res., 60:2163-2168, 2000). Briefly, tumor cells were treated with PBS, Ad-luc, Ad-mda7, Tocopherol, or a combination therefore for 72 h. At 72 h after treatment cells were washed with PBS, cells were collected into a tube by scraping and homogenized in a small glass homogenizer with a Teflon pestle (50 strokes on ice) in 100 μl of ice-cold buffer M(20 mM HEPES (pH 7.5), 10 mM KCl, 1.5 mM MgCl2, 1 mM EGTA, 1 mM EDTA, 1 mM DTT, 250 mM Sucrose, 0.1 mM PMSF, 2 μg/ml pepstatin, 2 μg/ml leupeptin, and 2 μg/ml aprotinin). The homogenates were spun at 16,000×g for 20 min at 4° C., and the supernatant (mitochrondrial fraction) and the cell pellet (cytoplasmic fraction) was used for detecting cytochrom c by western blot analysis.

B. Results

1. Growth Inhibtion of Ovarian Cancer Cells is Enhanced by Treatment with Ad-mda7 in Combination with Vitamin E (Tocopherol)

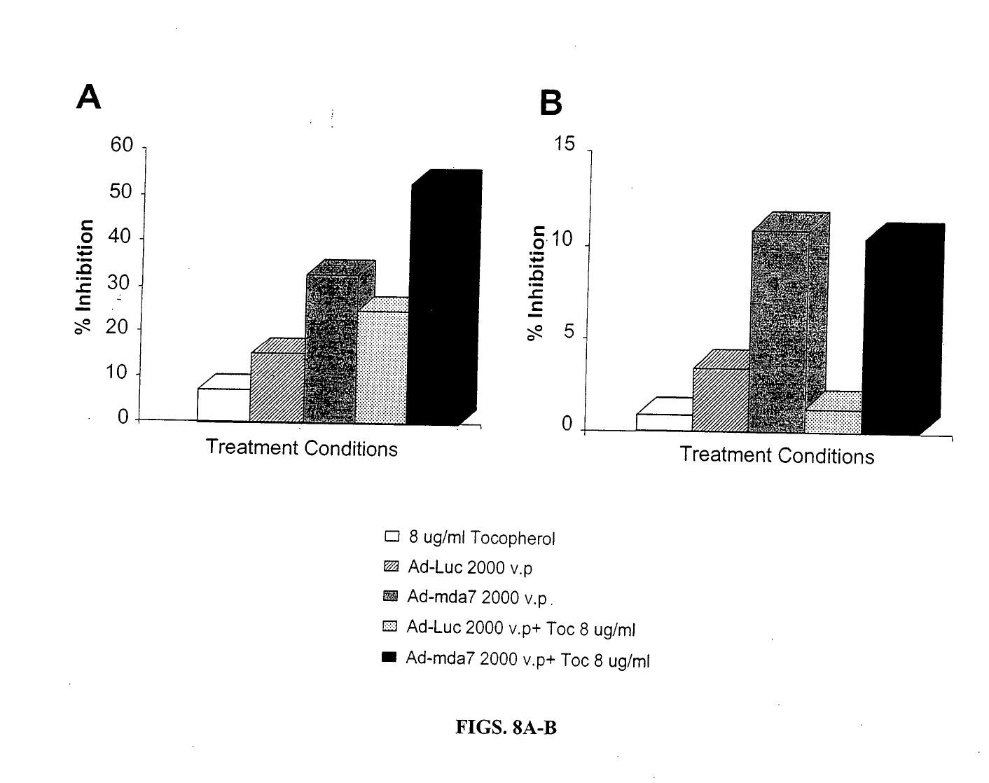

To determine whether the Ad-mda7 growth inhibition effect could be enhanced by Vitamin E, cells were treated with Ad-luc (vector control), Tocopherol (Vitamin E succinate), Ad-mda7 or a combination thereof. As shown in FIG. 8A, the combination of Ad-mda7 with Vitamin E succinate improved growth inhibition as compared to treatment with Ad-mda7 or Vitamin E succinate alone. FIG. 8B demonstrates that treatment of normal fibroblast cells with Ad-mda7 and Vitamin E does not increase growth inhibtion beyond what is observed after treatment with Ad-mda7 alone.

2. Indications that Apoptosis is Increased in Ovarian Cancer Cells Treated with Ad-mda7 and Vitamin E (Tocopherol)

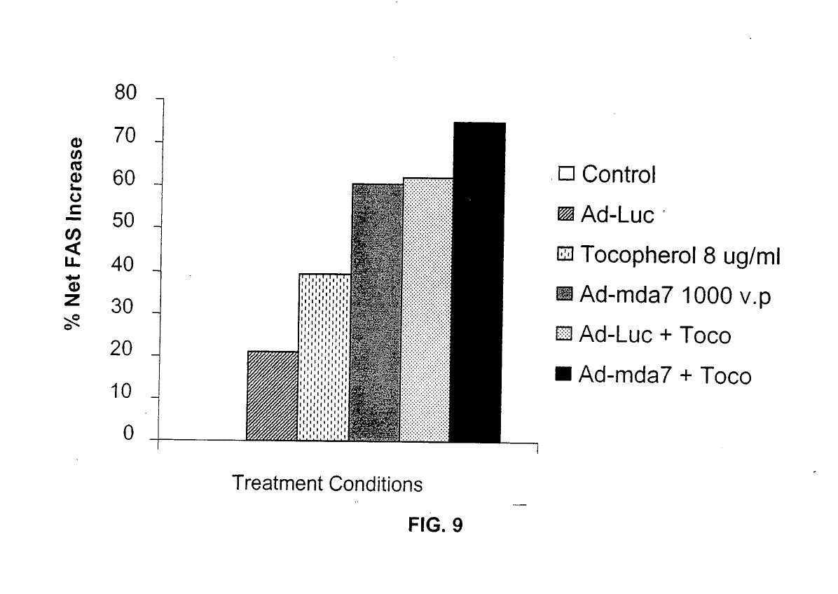

To determine if the improved growth inhibition effect of Ad-mda7 in combination with Vitamin E succinate might be attributable to apoptosis, Western blot analysis was performed on human ovarian cancer cells treated with Ad-luc, Ad-mda7, Vitamin E succinate or a combination thereof. This analysis revealed that production of MDA-7 protein is greatly enhanced in the presence of Vitamin E succinate. Consistent with the increased production of MDA-7 protein, Western blot analysis also revealed enhanced observation of hallmarks of apoptosis in cancer cells treated with Ad-mda7 in combination with Vitamin E succinate. Specifically, cleavage of Caspase-3, Caspase-8, Caspase-9, PARP, and Bid was observed to a greater degree in cancer cells treated with Ad-mda7 and Vitamin E succinate when compared to cells treated with either reagent alone. In addition, the enhanced release of cytochrome C from the mitochrondria as indicated by the decreased cytochrome C protein expression also indicated an increase in apoptotis in cells treated with Ad-mda7 and Vitamin E succinate. Finally, the apoptosis-associated Fas protein was more readily detected in cancer cells treated with Ad-mda7 and Vitamin E succinate than with either reagent alone (FIG. 9).

### Example 5

### Local and Systemic Inhibition of Lung Tumor Growth after Liposome Mediated mda-7/IL-24 Gene Delivery

A. Materials and Methods

1. Materials

All lipids (DOTAP, cholesterol) were purchased from Avanti Polar Lipids (Albaster, Ala.). Ham's/F12 medium and fetal bovine serum (FBS) were purchased from GIBCO-BRL-Life Technologies (New York, N.Y.). Polyclonal rabbit anti-human MDA-7 antibody was obtained from Introgen Therapeutics, Inc. (Houston, Tex.) and antimouse CD31 from Santa Cruz Biotechnology, Inc. (Palo Alto, Calif.).

2. Cell Lines and Animals

Human non-small cell lung carcinoma cell line A549 was obtained from American Type Culture Collection and maintained in Ham's-F12 medium supplemented with 10% FBS, 1% glutamate, and antibiotics. Murine UV2237M cells were obtained from Dr. Isaiah J. Fidler (M. D. Anderson Cancer Center) and maintained as described elsewhere (Ramesh et al., 2001). Cells were regularly passaged and tested for presence of mycoplasma. Four- to six-week-old female BALB/c nude (nu/nu) mice (Harlan-Sprague Dawley Inc., Indianapolis, Ind.) and C3H/Ncr mice (National Cancer Institute, Fredericksburg, Md.) used in the study were maintained in a pathogen-free environment and handled according to institutional guidelines established for animal care and use.

3. Purification of Plasmids

The plasmids used in the study were cloned in pVax plasmid vector (Invitrogen, Carlsbad, Calif.) and purified as described elsewhere (Templeton et al., 1997; Gaensler et al., 1999). Briefly, plasmids carrying the bacterial β-galactosidase (Lac-Z), chloramphenicol acetyl transferase (CAT), or human mda-7 cDNA, under the control of cytomegalovirus (CMV) promoter, were grown under kanamycin selection in the Escherichia coli host strain DH5α. Endotoxin levels of purified plasmids were determined by using the chromogenic limulus amebocyte lysate kinetic assay kit (Kinetic-QCL; Biowhittaker, Walkersville, Md.). The concentration and purity of the purified plasmid DNA's were determined by OD 260/280 ratios.

4. Synthesis of DOTAP:Chol Liposomes and Preparation of DOTAP:Chol-DNA Mixtures

DOTAP:Chol liposomes were synthesized and extruded through Whatman filters (Kent, UK) of decreasing size (1.0, 0.45, 0.2, and 0.1 μm) as described elsewhere (Chada et al., 2003; Templeton et al., 1997). DOTAP:Chol-DNA complexes were prepared fresh two to three hours prior to injection in mice.

5. Particle Size Analysis

Freshly prepared DOTAP:Chol-DNA complexes were analyzed for mean particle size by using the N4 particle size analyzer (Coulter, Miami, Fla.). The mean particle size of the liposome-DNA complexes ranged between 300 nm and 325 nm.

6. Effect of DOTAP:Chol-mda7 Complex on Subcutaneous Tumor Xenografts

In all the experiments, 5×10⁶ tumor cells (A549) suspended in 100 μl sterile phospate-buffered saline (PBS) were injected into the right dorsal flank. When the tumors reached a size of 4-5 mm², the animals were randomized into groups and treatment was initiated. Tumor-bearing animals were divided into four groups of six animals. Group 1 received no treatment, group 2 received PBS, group 3 received DOTAP:Chol-LacZ complex (50 μg/dose), and group 4 received DOTAP:Chol-mda-7 complex (50 μg/dose); all treatments were administered intratumorally and were given daily for a total of six doses. Animals were anesthetized with methoxyflurane (Schering-Plough, Kenilworth, N.J.) for intratumoral injections per institutional guidelines. Tumor measurements were recorded every other day by observers without knowledge of the treatment groups, and tumor volumes were calculated by using the formula V (mm³)=a×b²/2, where “a” is the largest dimension and “b” is the perpendicular diameter (Saeki et al., 2002; Ramesh et al., 2001). Antitumor efficacy data are presented as cumulative tumor volumes for all animals in each group to account for both size and number of tumors. In all experiments, the statistical significance of changes in tumor size was determined on days 21, and 24 by ANOVA

To test the effect of mda-7 on mouse tumor cells, we utilized a syngeneic tumor model. For this purpose, C3H mice were injected subcutaneously with murine UV2237m fibrosarcoma cells (1×10⁶) and divided into three groups (n=8/group). When the tumor size reached 4-5 mm²⁻, animals received intratumoral treatment as follows: no treatment (control), DOTAP:Chol-CAT complex, or DOTAP:Chol-mda-7 complex. Treatment schedule and analyses of the therapeutic effects were the same as already described for the A549 tumor model. Experiments were repeated two times for statistical analysis and significance calculated on days 21, and 23 by ANOVA.

7. Measurement of MDA-7, Apoptosis, and CD31

Subcutaneous A549 or UV2237m tumors established in nu/nu or C3H mice respectively were harvested and fixed in 4% buffered formalin, embedded in paraffin, and cut in 4-μm sections. Tissue sections were immunostained for MDA-7 transgene expression as described elsewhere (Saeki et al., 2002; Ramesh et al., 2003). The tumor cells staining positive for MDA-7 were analyzed under bright-field microscopy and quantitated by observers without knowledge of the treatment groups. At least five fields per specimen were analyzed. To determine the fate of tumor cells following treatment, sections of tumors were stained for apoptotic cell death with terminal deoxynucleotide transferase (Tdt) kit (Boehringer Mannheim, Indianapolis, Ind.) and counterstained with methylene blue or methyl green as described previously (Saeki et al., 2002; Ramesh et al., 2001). In all staining procedures, appropriate negative controls were included.

For CD-31 staining, tissues were stained with anti-CD31 antibody as described previously (Saeki et al., 2002; Ramesh et al., 2003) and observed under microscope in a blind fashion. Microvessel density (MVD) was determined semiquantitatively by counting the number of CD31 positive staining vessels in 5 randomly selected fields per tumor tissue under high power magnification (×400). A total of 15 fields representing 3 tumor tissues per treatment group was examined and quantitated and the results represented as the average number of vessels per field.

8. Tumor Characteristics after Treatment

To determine the therapeutic effects of the mda-7 gene, tumors were harvested from mice after the last treatment and subjected to histopathologic examination. Analysis was done by a pathologist without prior knowledge of the treatment groups.

9. Effect of DOTAP:Chol-mda7 Complex on Experimental Lung Metastasis

To test the effect of DOTAP:Chol-mda-7 complex on lung metastases, female nude mice were injected via tail vein with 10⁶ A549 tumor cells suspended in 100 μl of sterile PBS. Six days later, the mice were divided into three groups and treated as follows: no treatment (group 1), DOTAP:Chol-CAT complex (group 2), and DOTAP:Chol-mda-7 complex (group 3). There were eight mice in each group. All treatments comprised 50 μg liposome-DNA complex and were administered daily via tail vein using a 27-gauge needle for a total of six doses. Three weeks following the last dose, animals were euthanized by CO₂ inhalation. The lungs of each mouse were injected intratracheally with India ink and fixed in Feketes solution (Ramesh et al., 2001). The therapeutic effects of systemic mda-7 gene treatment were determined by counting the number of metastatic tumors in each lung under a dissecting microscope, by an observer without knowledge of the treatment groups. The data were analyzed, and differences among groups were interpreted as statistically significant if the P value was <0.05 by the Mann-Whitney rank-sum test.

As a syngeneic lung tumor model, C3H mice were injected with murine UV2237m fibrosarcoma cells (1×10⁶) and divided into three groups (n=7/group). Six days after injection, animals were treated as follows: no treatment, DOTAP:Chol-CAT complex, or DOTAP:Chol-mda-7complex. Treatment schedule and analyses of therapeutic effect were the same as already described for the A549 models. Experiments were performed two times for statistical significance.

B. Results

1. In Vitro Ttransfection of Tumor Cells with DOTAP:Chol-mda-7 Complex

The ability of DOTAP:Chol liposomes to deliver plasmid DNA into human (A549) and mouse (UV2237m) tumor cells by using expression plasmids encoding the human MDA-7/IL-24 protein was evaluated. Transfection with DOTAP:Chol liposomes complexed with mda-7 plasmid DNA, resulted in expression of exogenous MDA-7 protein in both A549 and UV2237m tumor cells at 24 and 48 h. MDA-7 expression was not observed in PBS treated control cells. Analysis of tissue culture supernatant from DOTAP:Chol-mda-7 transfected A549 and UV2237m cells showed secreted MDA-7 protein at 48 h but not at 24 h. Detection of secreted MDA-7 protein at 48 h is unlike that observed in Ad-mda7 treated cells where secreted MDA-7 protein is detectable at 24 h (Mhashilkar et al., 2001). This suggests that the transgenic MDA-7 expression achieved using DOTAP:Chol. liposome is less than that obtained with Ad-mda7. Secreted MDA-7 protein was not observed in PBS treated cells. Thus, DOTAP:Chol liposomes could effectively deliver mda-7 DNA to tumor cells resulting in intracellular and secreted transgenic MDA-7 production albeit less than Ad-mda7.

2. MDA-7 Inhibits Subcutaneous Tumor Growth

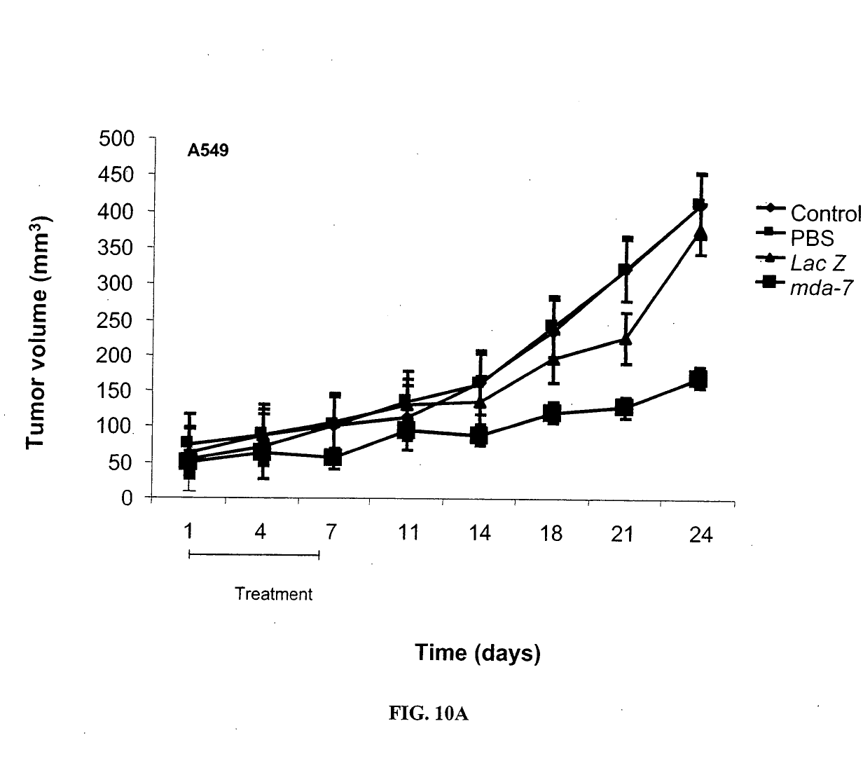

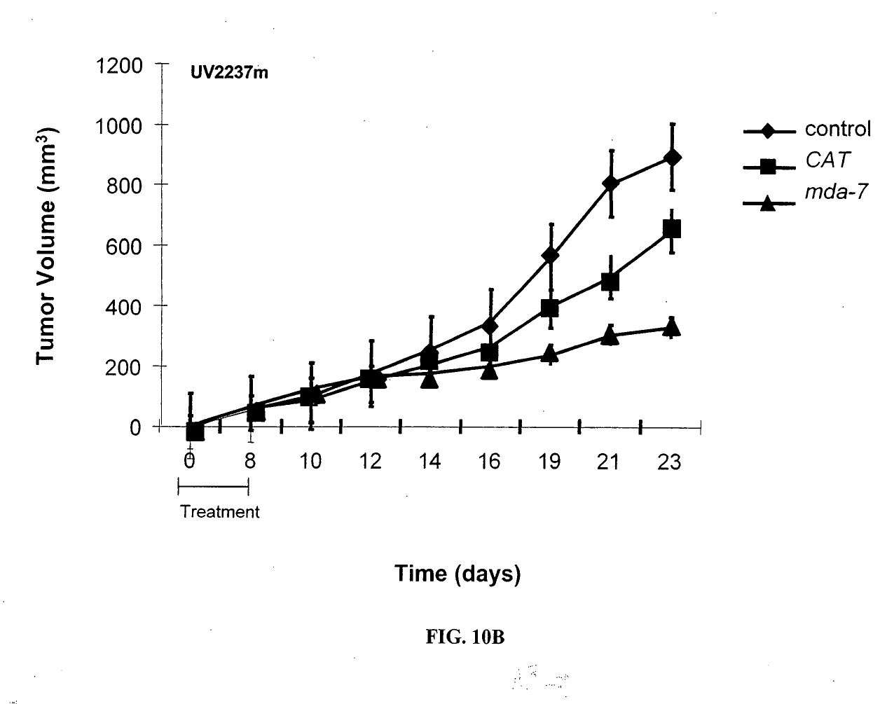

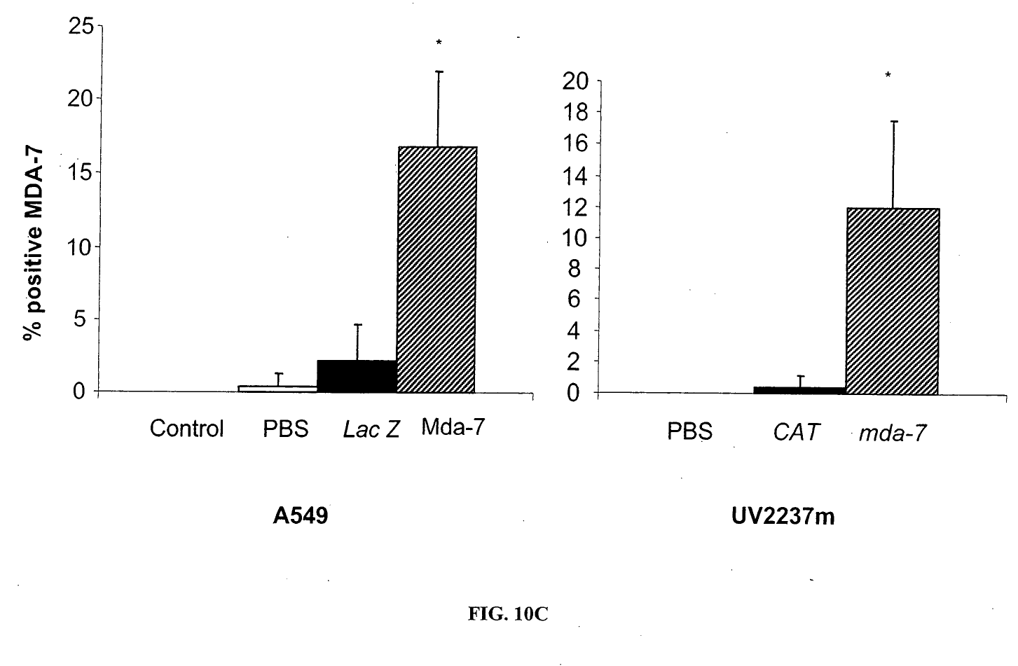

The ability of the DOTAP:Chol-mda-7 complex to suppress the growth of A549 human lung subcutaneous tumors in nu/nu mice was assessed. Treatment of tumor-bearing mice with the DOTAP:Chol-mda-7 complex via the intratumoral route significantly inhibited tumor growth (P=0.001) as compared with tumor growth in animals that were untreated, treated with PBS, or treated with DOTAP:Chol-LacZ complex (FIG. 10A). Histopathological analysis of the tumors revealed no significant changes in the tumor infiltrating cells among the various treatment groups.

The therapeutic effects of the mda-7 gene on subcutaneous murine tumors in C3H mice were next evaluated. Mice bearing UV223M tumors were divided into three groups, one receiving no treatment, a second receiving treatment with DOTAP:Chol-CAT complex, and a third receiving treatment with the DOTAP:Chol-mda-7 complex. Growth of UV2237m tumors was inhibited starting from day 19 in mice treated with intratumoral administration of the DOTAP:Chol-mda-7 complex when compared with tumor growth in the two control groups (FIG. 10B). However significant tumor inhibition was observed on day 23 (P=0.01). Tumor inhibition (P=0.24) was also observed in mice treated with DOTAP:Chol-CAT complex compared to untreated control mice. However, the inhibitory effect observed in DOTAP:Chol-CAT complex treated mice is attributed to non-specific effects and is in agreement with our previous studies (Ramesh et al., 2001).

To demonstrate that the observed tumor-suppressive effects was due to mda-7 gene expression, subcutaneous A549 and UV2237m tumors obtained at 48 hours after injection were subjected to immunohistochemical analysis for MDA-7 protein expression. MDA-7 protein expression was seen in 18% and 13% of A549 and UV223m tumors respectively that were treated with the DOTAP:Chol-mda-7 complex (P=0.001; FIG. 10C), a significantly higher number than in the animals that were not treated, treated with PBS, treated with DOTAP:Chol LacZ or treated with DOTAP:Chol-CAT complex. Some level of non-specific staining was observed in A549 tumors that were treated with DOTAP:Chol-CAT complex. Analysis of the pattern of MDA-7 expression revealed intense intracellular staining in addition to a more diffuse staining pattern that appeared to be extracellular. This pattern of staining was observed in both human tumor xenografts and murine syngeneic tumors.

3. Apoptotic Cell Death in Lung Tumors Ttreated with DOTAP:Chol-mda7 Complex

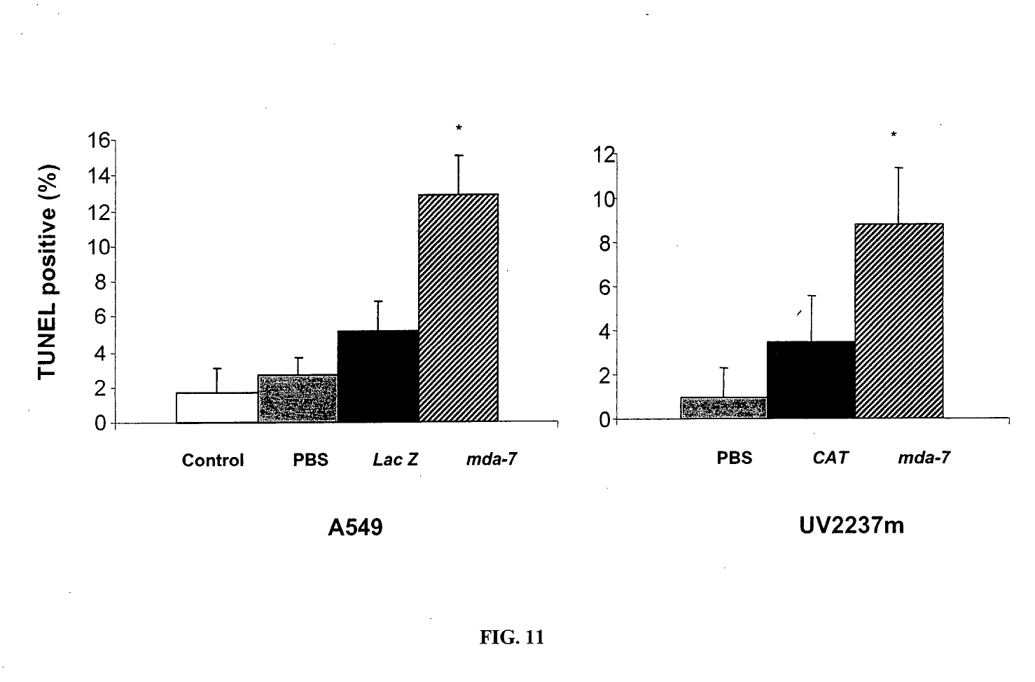

To determine the fate of tumor cells after treatment with the DOTAP:Chol-mda-7 complex, subcutaneous tumors (A549, UV2237m) from nu/nu mice and C3H mice were analyzed for apoptotic cell death as previously described (Saeki et al., 2002). A significant (P=0.001) level of TUNEL positive cells (13% A549, and 9% UV2237m) indicative of apoptotic cell death was observed in tumors treated with DOTAP:Chol-mda-7 compared to tumors from control animals that were untreated, treated with PBS, treated with DOTAP:Chol-CAT or treated with DOTAP:Chol-LacZ (FIG. 11).

4. Reduced CD31-Positive Staining in Lung Tumors Treated with DOTAP:Chol-mda-7 Complex

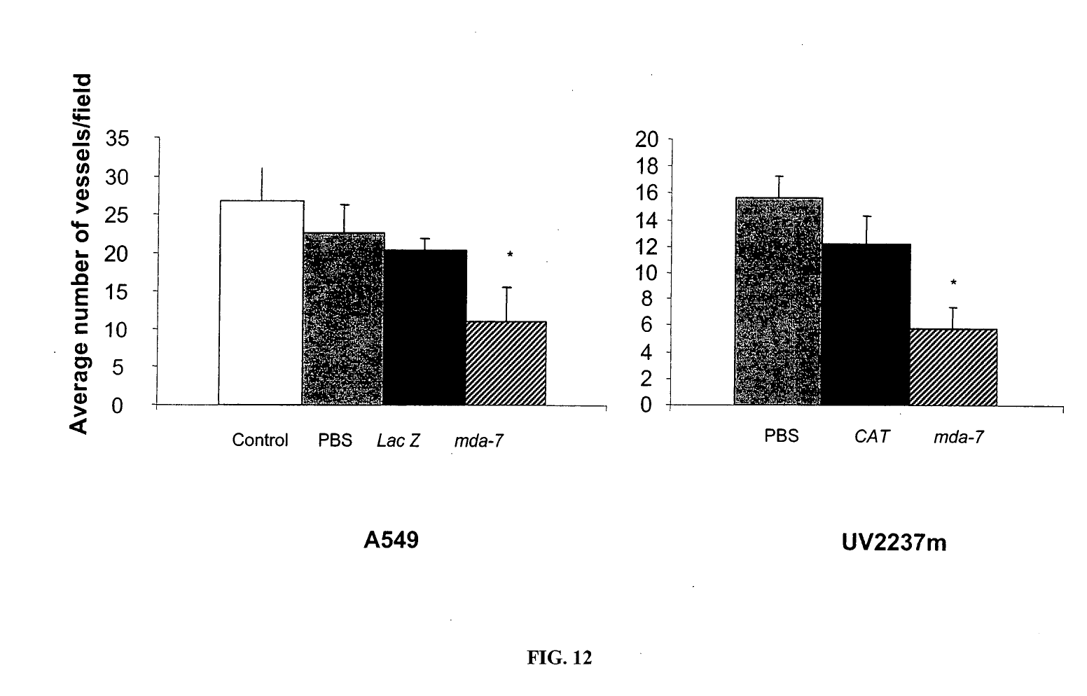

To determine the effect of mda-7 treatment on tumor vascularization, tumor tissues were subjected to CD31 staining as previously described (Saeki et al., 2002; Ramesh et al., 2003). Levels of CD31-positive staining was significantly (P=0.01) reduced in DOTAP:Chol-mda7 treated A549 (10%) and UV2237m (5.8%) tumor tissues compared to tumor tissues obtained from untreated, PBS-treated, DOTAP:Chol-LacZ complex treated, and DOTAP:Chol-CAT-treated mice (FIG. 12). Reduced CD31 staining is indicative of reduced vascularization.

5. MDA-7 Inhibits Experimental Lung Metastases

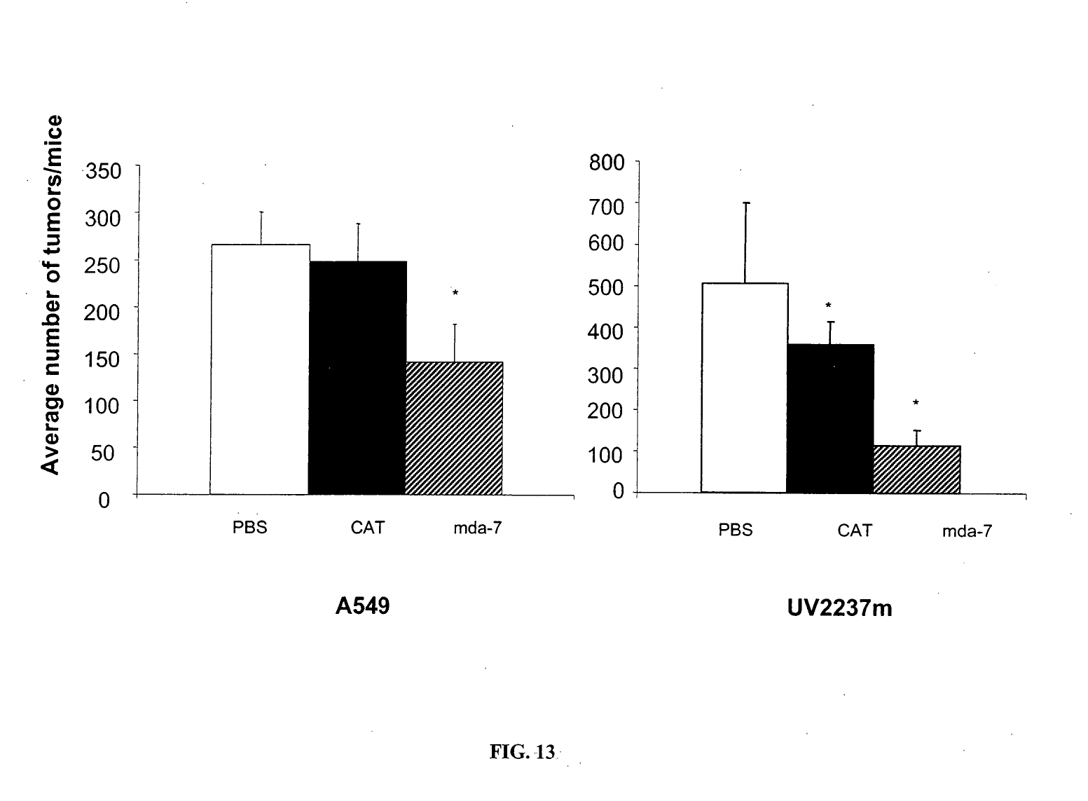

The activity of DOTAP:Chol-mda-7 complex was next investigated in an experimental lung metastases model using human A549 lung cancer cells or mouse UV227m cells. Intravenous delivery of tumor cells results in rapid tumor seeding of lungs, and animals succumb to overwhelming lung tumor burden after 30 days. Systemic treatment of A549 and UV2237m lung tumor-bearing nude or C3H mice with DOTAP:Chol-mda-7 complex resulted in a significantly (P<0.05) lower number of lung metastases than treatment with PBS or DOTAP:Chol-CAT complex (FIG. 13). In UV2237m mice, treatment with DOTAP:Chol-CAT complex resulted in a significant reduction in the number of tumor nodules when compared to those treated with PBS suggesting some non-specific antitumor activity (FIG. 13). Furthermore, the treatment was well tolerated with no treatment related toxicity observed as evidenced by lack of morbidity and mortality.

### Example 6

### Ad-mda7 Induces Chemosensitization of Ovarian Cancer Cells

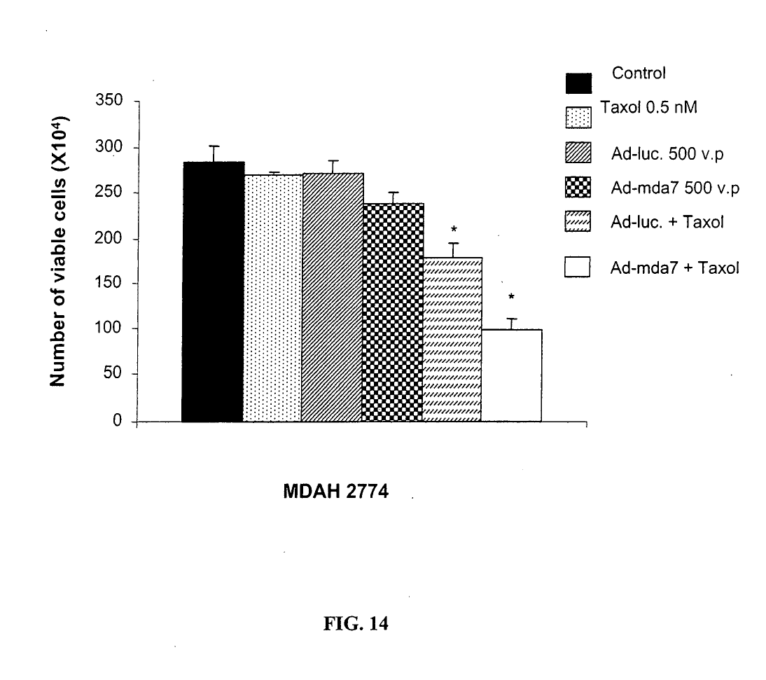

MDAH 2774 ovarian cancer cells seeded in 6-well culcutre plates (5×10⁵/well) were treated with Taxol (0.5 nM), Ad-luc (500 vp/cell), Ad-luc and Taxol or Ad-mda7 and Taxol. Cells were harvested at 72 hours after treatment and analyzed for cell viability by trypan blue exclusion assay. Untreated cells served as controls. Cells treated with Ad-luc and Taxol or Ad-mda7 and Taxol showed growth inhibition compared to other treatment groups (FIG. 14). However, significant growth inhibition that was additive to synergistic was observed only in cells that were treated with Ad-mda7 and Taxol (P=<0.05) (FIG. 14). Experiments were conducted in triplicate wells and the results represented as the average of two separate experiments.

### Example 7

### mda-7 Gene Transfer Sensitizes Breast Carcinoma Cells to Chemotherapy, Biologic Therapies and Radiotherapy: Correlation with Expression of blc-2 Family Members

A. Materials and Methods

1. Cells and Reagents

All cell lines were obtained from American Type Culture Collection (ATCC, Rockville, Md.). The breast cancer cell lines evaluated were T47D, MCF-7, MDA-MB-453, SKBr3, MDA-MB-231, MDA-MB-468, MDA-MB-361, HBL-100 and BT-20. Primary human mammary epithelial cells (HMEC), human vascular endothelial cells (HUVEC) and MJ-90 human fibroblasts were obtained from Clonetics (San Diego, Calif.). The cells were grown in DMEM medium (GIBCO, Grand Island, N.Y.) and fetal bovine serum (5-10%, according to each cell line), and routinely tested for mycoplasma. Herceptin (Genentech, San Francisco, Calif.), Taxotere (Aventis-RPR, Collegeville, Pa.), Tamoxifen (Sigma-Aldrich, St Louis, Mo.), and Adriamycin (Adria Labs, Columbus Ohio) were obtained from the MD Anderson Cancer Center pharmacy.

2. Recombinant Adenovirus

Production of the replication-deficient human type 5 Adenovirus (Ad5) containing the mda-7 gene (Ad-mda7), luciferase reporter gene (Ad-luc) or empty vector (Ad-CMVp(A)) have been previously reported (Mhashilkar, 2001). Construction of Ad-mda7 involved linking mda-7 cDNA to a CMV-IE promoter, followed by an SV40 polyadenylation [p(A)] sequence; this expression cassette was placed in the E1 region of Ad5. PCR™, restriction endonuclease digestion, and DNA sequencing analyses were used to verify virus stocks.

Western Blot analysis Cell lysates were subjected to 10% SDS polyacrylamide gel electrophoresis and analyzed by Western Blot, using the Super-Signal substrate for Horse-Radish Peroxidase (Pierce, Inc.). Polyclonal and monoclonal antibodies against MDA-7 were produced by Introgen Therapeutics. Other monoclonal antibodies used in the study recognized PKR, p53, BCL-2, BCL-XL, BAX, α-tubulin and β-actin (Santa Cruz Biotechnology). Secondary antibodies were purchased from Santa-Cruz Biotechnology (Santa Cruz, Calif.) and from Amersham Biosciences (Piscataway, N.J.).

3. Transduction and Drug Treatments

Cells were transduced with Ad-mda7, Ad-empty or Ad-luc with increasing multiplicities of infection (MOIs) in the presence or absence of the drugs as indicated (Tamoxifen 1-10 μg/mL; Taxotere 1-10 ng/mL; Adriamycin 1-10 ng/mL and Herceptin 1 μg/mL). Cells were plated at 500-2000 cells/well in 96-well format for ³H-thymidine incorporation-assays, or at 10⁵-10⁶ cells/well in a 6-well plate format for protein expression, trypan blue viability or apoptosis assays. In drug combination studies, drugs were added once just prior to addition of vectors and cells were continuously exposed to agents.

4. Cell Proliferation Analyses

Growth inhibition of cells was measured by ³H-Thymidine incorporation into the DNA of actively replicating cells. ³H-Thymidine was added to the cells (1 μCi/mL) and the reaction stopped 15 hours later by removal of the supernatant from recipient cells. The cells were harvested using Trypsin/EDTA (GIBCO), collected on a Filter using a Packard Filtermate cell Harvester, and washed in deionized water and methanol following the manufacturer protocols. The filters were dried and analyzed using a Matrix 9600 (Packard).

5. Apoptosis and Cell Viability Assays

Apoptosis was measured by TUNEL and Annexin V assays. For TUNEL assays, tumor sections were analyzed for apoptosis using the Chromogenic TUNEL-POD (Roche Diagnostics, Indianapolis, Ind.) assay following manufacturer protocols (Saeki et al., 2000), and TUNEL positive cells identified by their dark brown staining. Annexin V assays were done using the ApoAlert Annexin V-FITC kit (CLONTECH, Palo Alto, Calif.) (Saeki et al., 2000). Cell viability was determined by trypan-blue exclusion assay.

Cell cycle analysis with Propidium Iodide (PI) staining. Cell-cycle progression was determined by PI staining of cellular DNA. Cells were prepared as a single cell suspension of 1-2×10⁶ cells/mL of PBS, fixed with cold 70% ethanol for 2 hours, and centrifuged. The fixative was decanted, and the cells washed in PBS, and stained with Propidium Iodide (PI, 50 μg/mL) and RNAse (20 μg/mL in PBS). Treated cells were evaluated by FACS analysis.

Clonogenic survival assays. Clonogenic survival assays were performed using Ad-mda7 and Ad-CMVp(A) (MOI 2000 viral particles/cell) in combination with XRT (0, 2 or 4 Gy). MDA-MB-468 breast cancer cells were cultured at 1×10⁵ cells with empty adenovirus vector (Ad-CMVp(A)) or Ad-mda7 for 2 days and then treated with radiation therapy (XRT). Cells were reseeded at a density of 2.5×10⁵ cells. Clonogenic survival was assessed 3 weeks later; colonies were fixed, stained with Giemsa, and counted (Nishikawa et al., 2004).

6. Animal Studies and Immunohistochemical Analysis

Balb C nu/nu mice were obtained from Charles River Laboratories (Wilmington, Mass.). Six-week old female mice were injected with breast cancer cells into the right hind limb and observed for tumor growth. In all the experiments, tumor cells (MDA-MB-361; MCF-7 or MDA-MB-468) suspended in 100 μl sterile phosphate buffered saline (PBS) were injected into the right dorsal flank and allowed to grow to approximately 100 mm³. Animals were then randomized into groups (n=5-10 animals/group) and treatment initiated as follows: Group 1 received PBS, Group 2 received Ad-luc and Group 3 received Ad-mda7. Dosing schedules varied for different models: in the MB-361 xenograft model, when tumors reached 130 mm³, 10 animals per group were injected with PBS, Ad-luc or Ad-mda7 at doses of 1×10¹⁰ vp on alternate days for a total of three doses. In the MDA-MB-468 model, five animals each with tumors of 130 mm³ were injected with PBS, Ad-luc or Ad-mda7 at a dose of 2×10¹⁰ vp on alternate days for a total of three injections. In MCF-7 xenograft models, 10 animals per group were injected when tumors reached 85 mm³ with: PBS, Ad-luc or Ad-mda7 at 1×10¹⁰, 3×10¹⁰ or 1×10¹¹ vp on alternate days for six injections

For evaluation of radiotherapy combinations, mice were divided into six treatment groups (n=5 per group): Phosphate buffered saline (PBS), Ad-luc, Ad-luc+XRT, XRT alone, Ad-mda7, Ad-mda7+XRT. The tumors were treated by direct injection with PBS or adenoviral vectors at a dose of 2×10¹⁰ vp/ml on days 1, 3, and 5. On day 6, the animals were treated with a single application of radiation (5 Gy) to the hind limb. Tumor measurements were taken every other day and the volume was calculated. The tumors were harvested from selected mice and embedded in paraffin immediately after the animals were sacrificed. Tumor samples were analyzed for MDA-7 and PKR expression by immunohistochemistry using the BCIP/NBT substrate kit (Vector Laboratories, Burlingame, Calif.). Intratumoral injections were performed under anesthesia using methoxyflurane (Schering Plough, Kenilworth, N.J.) as per institutional guidelines. Tumor measurements were recorded every other day without knowledge of the treatment groups, and the volume was calculated using the formula V (mm 3)=a×b²/2, where “a” is the largest dimension and “b” is the perpendicular diameter (15,23). Mice were euthanized when the tumors reached 1.5 cm in size.

7. Statistical Analysis

The statistical significance of the experimental results was calculated using Student's t-test for tumor measurements. ANOVA and two-tailed Student's t-test were used for statistical analysis of multiple groups and pair-wise comparison, respectively, with p<0.05 considered significant.

B. Results

1. MDA-7 is Overexpressed in Breast Cancer Cells Following Treatment with Ad-mda7

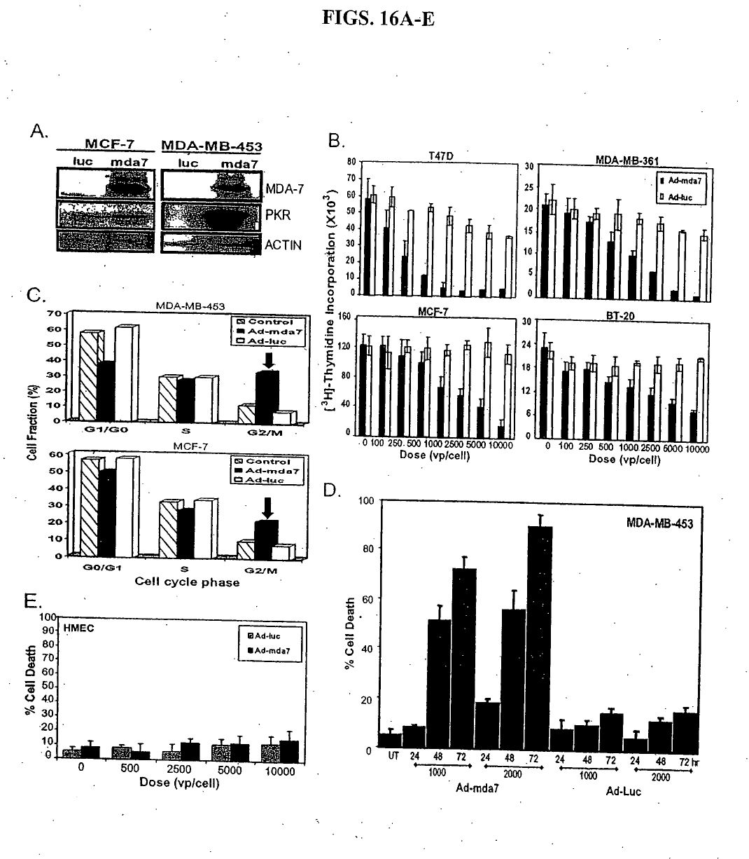

A panel of nine breast cancer cell lines was used; parental tumor types and p53 mutational status are summarized in FIG. 15 Western blot analysis of two representative breast carcinoma lines: MDA-MB-453 (mutant p53) and MCF-7 (wild type p53) shows that MDA-7 protein is markedly overexpressed after Ad-mda7 transduction, regardless of p53 status (FIG. 16A); MDA-7 protein was not evident in the lysates of Ad-luc or PBS treated cells. β-actin was used as an internal control to ensure equal protein loading. The results show that ectopic expression of MDA-7 induced high levels of the ds RNA activated ser/thr kinase PKR, while cells transduced with Ad-luc did not. Western blot analysis of lysates from additional breast cancer cells (T47D; MB-231; MB-361; MB-468; SKBr3; BT-20; HBL-100) also showed high expression of MDA-7 in Ad-mda7 treated cells.

2. Ad-mda7 Induces Cell Death in Breast Cancer Cells In Vitro

Breast cancer cell lines: MCF-7, T47D, SKBr3, HBL-100, BT-20, MDA-MB-231, MDA-MB-468, MDA-MB-453 and MDA-MB-361, and three normal cell types: HMEC (mammary epithelium); MJ90 (fibroblasts) and HUVEC (endothelial cells) were treated with increasing doses of Ad-mda7 or control vectors—Ad-luciferase (Ad-luc) or Ad-CMVp(A) (Ad-empty) and evaluated for growth inhibition. The vector concentration required for growth inhibition by 50% (IC₅₀) was calculated for each cell line and is listed in FIG. 15. The IC₅₀ values for growth inhibition by Ad-mda7 were divided by the IC₅₀ values obtained for control vectors, to generate a Selectivity Index (S.I.). The SI indicates the relative ability for cell growth inhibition by Ad-mda7 compared to control Ad vectors. While the SI values of normal cells all equal 1; those of breast tumor cells show that Ad-mda7 is >2 to >30-fold (average >8) more cytotoxic than controls (FIG. 15). Although the S.I. values span a large range, they demonstrate that mda-7 activity is selective for transformed cells, and expression of MDA-7 protein does not induce toxicity in normal cells. Another example of the tumor-cell selective effect of Ad-mda7 comes from ³H-Thymidine incorporation assays (FIG. 16A); our results show significant dose-dependent growth inhibition in T47D, BT-20, MDA-MB-361 and MCF-7 breast cancer cells transduced with Ad-mda7 (p<0.001). MDA-7 expression inhibited cell proliferation by up to 96%, while growth of Ad-luc-transduced cells was minimally altered. These results suggested that Ad-mda7 might induce cell cycle arrest, and thus we performed cell cycle analysis (PI staining) of untreated, Ad-luc and Ad-mda7 transduced MDA-MB-453 and MCF-7 cells. As shown in FIG. 16C, cells transduced with Ad-mda7 undergo cell cycle block, and a 2-3 fold increase in the fraction of G2/M cells compared to untreated or Ad-luc treated cells.

The kinetics and dose-response of cell death were evaluated, and representative data are shown for MDA-MB-453 cells (FIG. 16D): At low Ad-mda7 doses (1000 vp/cell: 50 pfu/cell) significant cell death (p<0.001) was observed at 2 days post treatment; at higher doses significant killing was observed at day 1, and increased with time. In sum, MDA-7 expression kills breast tumor cells in both a time- and dose-dependent manner, whereas Ad-luc shows only minor effects (FIG. 16D). In contrast, the corresponding normal cells, human mammary epithelial cells (HMEC) show no significant toxicity from Ad-luc and Ad-mda7, even at longer time points (FIG. 16E). Additional studies using normal fibroblasts and primary endothelial cells confirmed the lack of cytotoxicity against normal cells (FIG. 15).

3. Apoptosis Induction by Ad-mda7

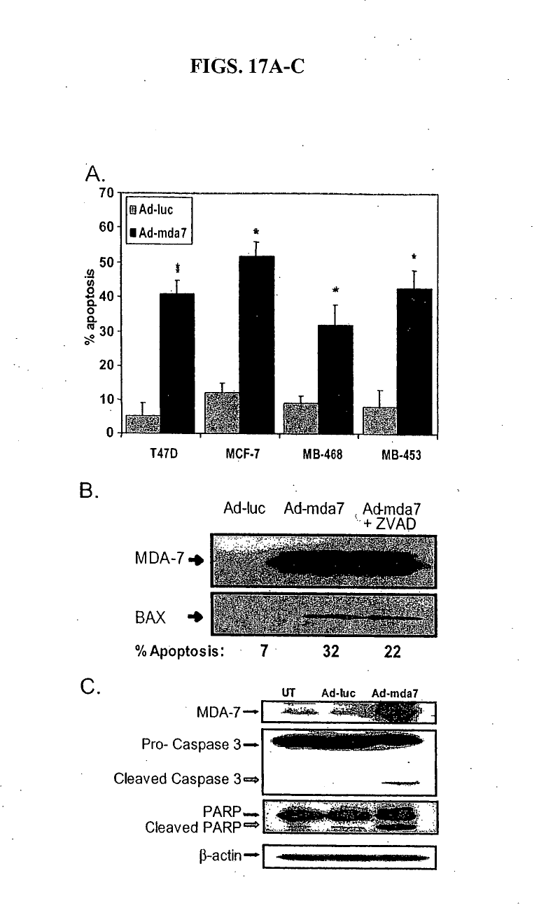

The expression of MDA-7 in tumor cells may affect signals other than those related to inhibition of cell growth, and is reported to activate apoptotic pathways in various cancer cell types (Mhashilkar et al., 2001; Su et al., 1998; Saeki et al., 2000; Chada et al., 2004). In agreement with these findings, transduction with Ad-mda7 was observed to trigger apoptosis in T47D, MCF-7, MDA-MB-453 and MDA-MB-468 cell lines (41, 52, 43 and 32% respectively) (FIG. 17A). In contrast, when these lines were transduced with control Ad-luc or Ad-empty, the number of cells undergoing apoptosis was comparable to that observed in vehicle treated cells. Further assays on these cell lines confirmed that Ad-mda7 induced apoptosis dose-dependently, and up-regulated BAX in T47D breast cancer cells (FIG. 17B). Treatment with the pan-caspase inhibitor ZVAD reduced apoptosis by 40%: however MDA-7 induction of BAX was not reduced. Treatment with Ad-luc did not activate BAX or apoptosis (FIG. 17B The mechanism of apoptosis induction was then evaluated. Ad-mda7 induced cleavage of caspase 3 and PARP, consistent with mitochondrial mediated apoptosis induction (FIG. 17C).

4. Expression of mda-7 Reduces Tumor Growth In Vivo

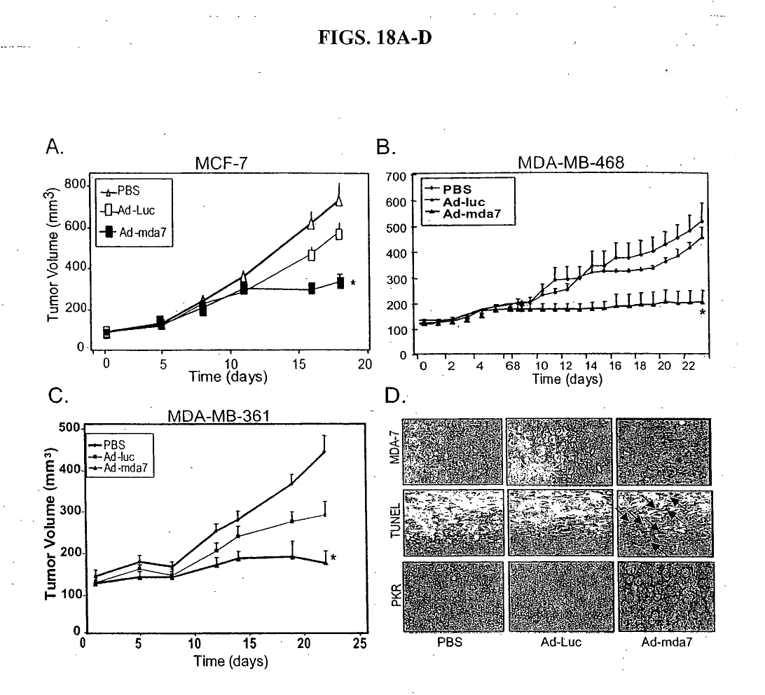

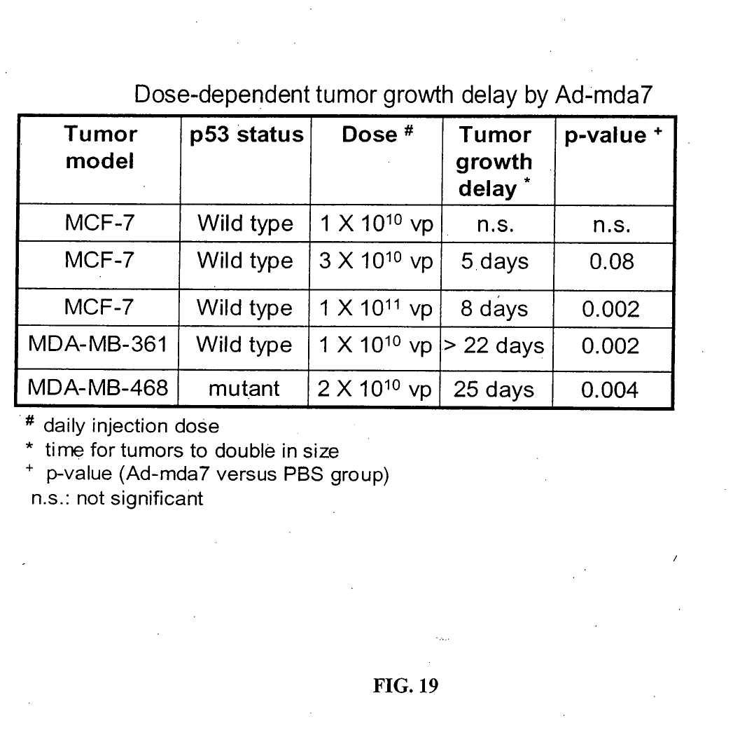

To determine if the in vitro growth inhibition of breast cancer cells induced by Ad-mda7 translated into a similar effect on tumor growth in vivo, we evaluated xenograft tumors generated using MDA-MB-361, MDA-MB-468 and MCF-7 cells in nude mouse models. When the tumors reached approximately 100 mm³, the animals were divided into treatment groups (n=5-10 animals per group), and treated with PBS, Ad-luc, or Ad-mda7. As shown in FIG. 18A-D, direct injection of the tumors with Ad-mda7 induced significant reduction of tumor volume, evident by day 10, on all 3 xenograft models (p=0.002-0.004). By day 20, control tumors treated with Ad-luc or PBS had undergone a 4- to 8-fold increase in volume, reaching a maximum of greater than 800 mm³. In contrast, Ad-mda7 treated tumors showed either minor tumor growth (MDA-MB-361 and MDA-MB-468) or grew to approximately 3-fold their initial volume (MCF-7). Ad-luc induced a variable effect on tumor growth, with maximal effect in the MB-361 model; however Ad-mda7 consistently induced much more robust and stable growth inhibition and resulted in prolonged tumor growth control in all three models. Significant growth inhibition was evident in both tumor cells that were mutant and wild type for p53 (see FIG. 19). A dose-escalation study was performed in p53 wild-type MCF-7 tumors, where it was found that low doses were not effective at blocking tumor growth, whereas a three-fold higher dose produced some tumor growth inhibition (p=0.08) and a log higher dose produced robust tumor growth inhibition of this aggressive tumor (p=0.002) (FIG. 19 and FIG. 18A-D). The rate of tumor growth was significantly reduced by Ad-mda7 treatment (see FIG. 18C), as reflected in the time required for these tumors to double in size (FIG. 19). These results show that expression of MDA-7 has anti-proliferative activity both in vitro and in vivo, in both p53 wild type and p53 mutant breast cancer cells. MDA-MB-468 xenografts were also analyzed for MDA-7 expression and apoptosis induction. Strong MDA-7 immunostaining was observed in Ad-mda7 treated, but not in PBS or Ad-luc treated tumors (FIG. 18D). TUNEL analysis showed that MDA-7 protein expression correlated with apoptosis. MDA-7 expressing tumors showed high expression of PKR protein (FIG. 18D), similar to that observed in vitro (FIG. 16A).

5. Combination of Ad-mda7 Transduction with Tamoxifen, Taxotere, or Adriamycin Treatments has an Additive Effect on Breast Tumor Cell Death

As shown above, single agent therapy with Ad-mda7 exhibits promising anti-tumor activity against breast cancer cells. However, current treatments for breast cancer employ a multi-modality therapeutic approach combining cytotoxic chemotherapies, radiation therapy, hormonal therapy and new biologic therapies, such as Herceptin (Winer et al., 2000; Fisher et al., 1997; Amat et al., 2003; Bonnadona, 1989; Tantivejkul et al., 2003; Pegram et al., 2004). To investigate if combining Ad-mda7 with chemotherapeutic agents could enhance cytotoxicity, breast cancer cell lines were treated with Ad-mda7 plus a series of chemotherapeutic agents, and analyzed using cell proliferation and cell viability assays. These cells were first treated with the estrogen antagonist Tamoxifen, a non-steroidal agent that acts by competing with estrogen for binding of its receptors in target tissue (Winer et al., 2000; Fisher et al., 1997). To facilitate evaluation of combinatorial drug interactions, sub-therapeutic doses of each agent were used.

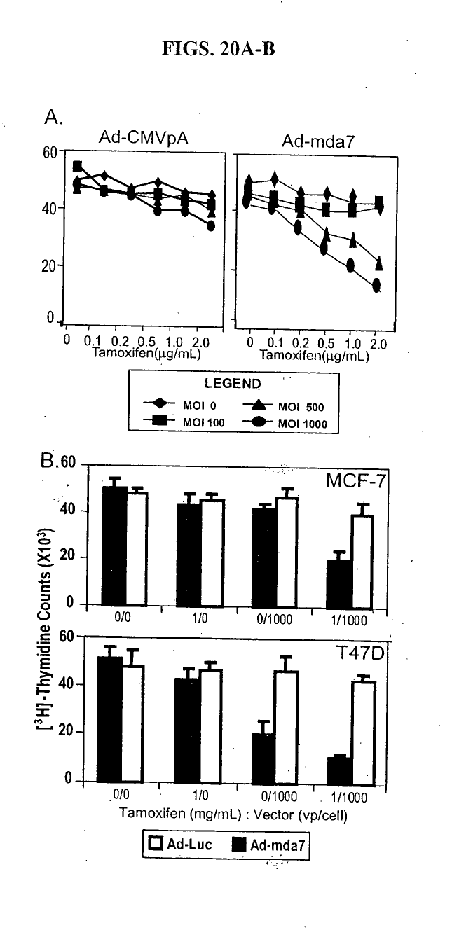

A dose-response between Ad-mda7 and Tamoxifen (FIG. 20A) was first established. Low doses of Ad-empty (0-1000 vp/cell) or Tamoxifen (up to 2 μg/ml) reduced cell proliferation by less than 20% in T47D cells. Addition of Ad-mda 7 at very low doses (100 vp/cell) did not affect cell growth while 500 vp/cell and 1000 vp/cell of Ad-mda7 resulted in dose-dependent growth inhibition when combined with Tamoxifen (60% and 80% inhibition, respectively). As shown in FIG. 20B (top panel), MCF-7 cells showed a minor response (<15%) to Tamoxifen (1 μg/mL) and Ad-mda7 (at 1000 vp/cell) alone, but treatment with a combination of both agents had a synergistic effect and reduced cell proliferation by more than 60% (p<0.001). Further studies were conducted to evaluate p53 mutant T47D cells: in this case treatment with Ad-mda7 as a single agent significantly reduced growth. However, as was the case with p53 wt MCF-7 cells, the effect of the two combined therapies was synergistic, and reduced thymidine counts by 80% (p<0.01). Transduction of tumor cells with Ad-luc or Ad-empty reduced growth by <15%. A similar synergistic interaction was also observed in MDA-MB-361 cells.

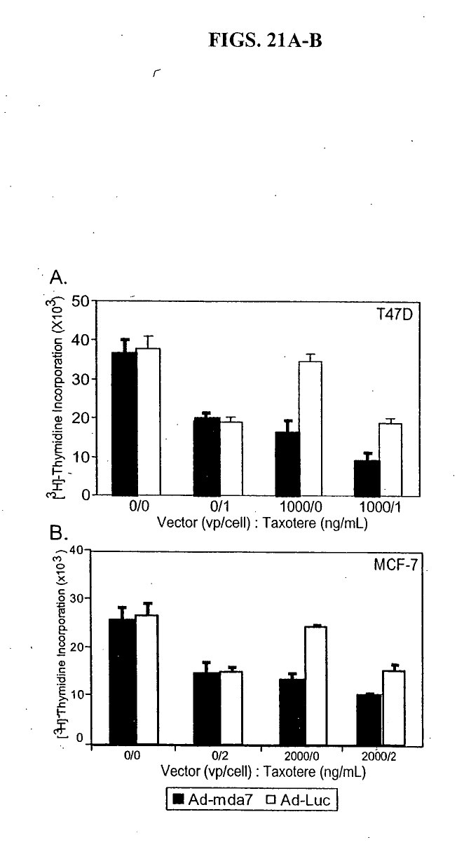

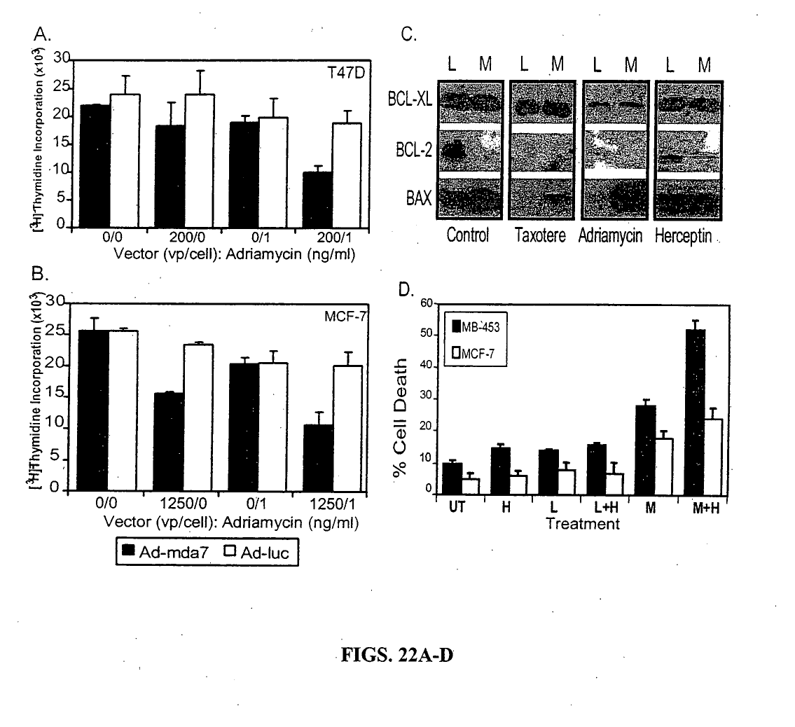

To investigate if Ad-mda7 could enhance the effects of agents that belong to the taxane family, breast cancer cells were treated with Taxotere (docetaxel, 0.5-2 ng/mL). Taxotere is an antineoplastic drug that disrupts the microtubule network, and prevents cells from completing mitosis and interphase (Winer et al., 2000; Fisher et al., 1997; Amat et al., 2003). Cell growth assays were performed on T47D and MCF-7 with doses of Taxotere that result in approximately 40% inhibition of thymidine incorporation (FIG. 21A-B). The cells were sensitive to Ad-mda7 but not Ad-luc; when Ad-mda7 was combined with Taxotere, enhanced sensitization was observed in both cell lines. Identical results were obtained in MDA-MB-361 cells. Adriamycin (doxorubicin), a cytotoxic anthracycline antibiotic, whose effects are thought to be mediated by its nucleotide base intercalation and cell membrane lipid binding activities, was then tested (Winer et al., 2000; Tantivejkul et al., 2003). A direct interaction of this agent with repair enzyme topoisomerase II, forming DNA-cleavable complexes is thought to play a role in this drug's cytotoxicity. Treatment of T47D or MCF-7 cells with Ad-mda7 (200-2500 vp/cell) or with Adriamycin (1 ng/mL)-used as single agents-decreased cell proliferation; while treatment with the combination of Ad-mda7 and Adriamycin demonstrated supra-additive (synergistic) effects (FIG. 22A-B). Synergistic activity was evident at low drug concentrations. In T47D cells, 200 vp/cell Ad-mda7 or 1 ng/ml Adriamycin resulted in minor (17-23%) reduction in cell growth, but the combination of both agents resulted in significantly greater (>60%) growth inhibition (p<0.01).

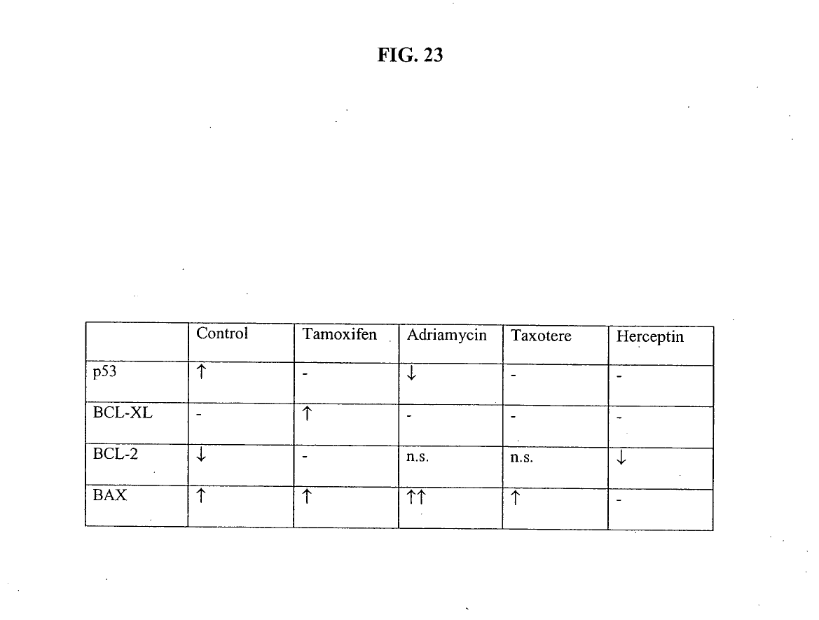

Apoptosis signaling (p53; BCL-XL; BCL-2 and BAX) in breast cancer cells treated with Ad-mda7 in combination with Taxotere, Adriamycin, Tamoxifen and Herceptin (FIG. 15C and FIG. 16) was then evaluated. Single agent Ad-mda7 treatment of breast tumor cells did not substantially alter steady state levels of p53 or BCL-XL, while it decreased expression of BCL-2 and up-regulated BAX (FIG. 22C), similar to the effect observed in T47D cells. In cells treated with Taxotere, Adriamycin or Herceptin, no significant alterations in p53 and BCL-XL were noted after Ad-mda7 treatment (FIG. 23). Steady-state levels of BCL-2 and BCL-XL were reduced in cells treated with both chemotherapies. Ad-mda7 induced up-regulation of BAX in cells treated with Taxotere or Adriamycin, whereas MDA-7 expression caused decreases in BCL-2 when combined with Herceptin (FIG. 22C). The molecular changes in p53 and BCL-2 family members are summarized in FIG. 23. These apoptotic mediators demonstrate differential regulation based upon the drug and vector used. Ad-mda7 consistently up-regulated BAX in breast cancer cells when delivered either as monotherapy or in combination with chemotherapies.

Herceptin is a humanized antibody developed as an antagonist for the human epidermal growth factor receptor-2 (HER-2) (Pegram et al., 2004). In this study, studies were conducted to evaluate the cytotoxicity of Herceptin on MDA-MB-453 (HER2 over-expressing) and MCF-7 (HER2 negative) cells enhanced by expression of Ad-mda7 (FIG. 22D). Indeed, a synergistic >5-fold increase in cell death of MDA-MB-453 cells compared to untreated controls was observed, when cells were transduced with Ad-mda7 combined with Herceptin (p<0.001). In contrast, MCF-7 cells were resistant to Herceptin, as predicted by their lack of Her-2 receptor expression. Ad-mda7 induced death in MCF-7 cells; however the combination of Ad-mda7/Herceptin did not show enhanced activity. Treatment with Ad-luc either as a single agent or in combination with Herceptin, under similar conditions as above, did not significantly increase the percentage of cell death observed. These results indicate that the combined use of Ad-mda7 and chemotherapeutic, hormonal or biologic agents can lower the treatment concentrations required for tumor cell death compared to monotherapy treatment, and thus potentially reduce the associated toxicities.

6. Combination of Ad-mda7 with Radiation Therapy has a Synergistic Effect on Breast Cancer Cells Clonogenic Survival and Tumor Growth.

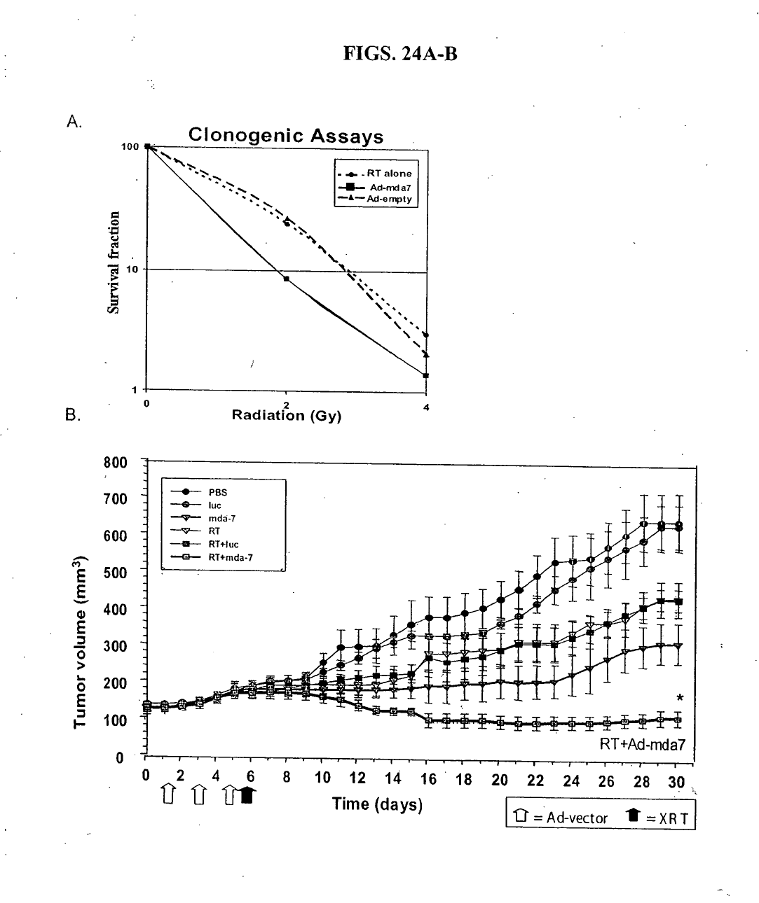

Studies were then conducted to investigate the impact of Ad-mda7 and XRT combination therapy on MDA-MB-468 breast cancer cells. MDA-MB-468 cells were treated with an empty adenoviral vector (Ad-p(A)) or Ad-mda7 (both at a MOI of 2000 vp/cell), and irradiated. Anti-tumor activity was evaluated using a colony formation assay. As shown in FIG. 24A, a significant decrease in survival of the Ad-mda7+XRT treated cells compared to XRT or Ad-p(A) was observed. MDA-7 mediated enhancement of tumor cell killing was observed at both 2 and 4 Gy XRT (FIG. 24A). These results show that the combination of Ad-mda7 plus radiation therapy inhibits colony formation in MDA-MB-468 cells, in vitro, in a supra-additive manner.

To investigate the effects of the combined Ad-mda7 and XRT therapy, in vivo, we treated large (>130 mm³) MDA-MB-468 breast cancer xenograft tumors with Ad-mda7, Ad-luc or PBS individually or combined with XRT (5 Gy) The animals were divided into six treatment groups (n=5 in each group), and treated with PBS, Ad-luc, Ad-luc+XRT, XRT, Ad-mda7 or Ad-mda7+XRT. Marked differences in tumor size between the treatment groups were evident within one week after treatment cessation. Treatment with XRT alone, Ad-mda7 alone, and the combination of Ad-luc and XRT all resulted in significant decreases in tumor growth (p<0.05). However, the most marked change was seen in the animals that received the combination of XRT and Ad-mda7. As shown in FIG. 24B, a substantial inhibition of tumor progression in the Ad-mda7-treated animals was observed. However regression in tumor growth was observed when Ad-mda7 and XRT were used in combination (p=0.0017). Tumors from Ad-mda7/XRT treated animals initially increased in size by approximately 50%, but subsequently regressed by up to 80%. All of the animals in the Ad-mda7/XRT group underwent regression, with tumor measurements reaching a nadir of <42 mm³. Control tumors increased in size by more than 500%, whereas XRT treated tumors increased by >350%. Tumors in the Ad-mda7/XRT group exhibited prolonged stabilization, whereas the XRT, Ad-luc or Ad-luc/XRT tumors grew progressively larger. When evaluated for time to tumor size doubling, PBS and Ad-luc treated animals reached this size by day 10 and 11 respectively; animals in both XRT and Ad-luc/XRT groups took 16 days whereas Ad-mda7 treated tumors took 25 days. Tumors from Ad-mda7/XRT treated animals had not doubled in size by >30 days, and averaged 25% smaller than their starting size. Elevated expression of transgenic MDA-7 protein was observed only in Ad-mda7-treated tumors, but not in PBS- or Ad-luc-treated tumors (FIG. 18D).

### Example 8

### Human Interleukin 24 (IL-24) Protein Kills Breast Cancer Cells Via the IL-20 Receptor and is Antagonized by IL-10

A. Materials and Methods

1. Cell Culture and Reagents

MDA-MB231 and MDA-MB453 breast cancer lines were obtained from the America Type Culture Collection (ATCC, Manassas, Va.) and were maintained in DMEM (Hyclone, Inc., Logan, Utah) supplemented with 10% fetal bovine serum (Life Technologies, Inc.), 100 units/ml penicillin, 100 μg/ml streptomycin, 2 mM L-glutamine, and HEPES buffer (Life Technologies, Inc., Grand Island, N.Y.). The cells were screened routinely to verify lack of mycoplasma contamination and were used in the log phase of growth. Monoclonal anti-IL-24 antibody was prepared as described previously (Caudell et al., 2002). Rabbit phospho-Stat3 (Tyr705) antibody was purchased from Cell Signaling Technology Inc. (Beverly, Mass.), β-Actin monoclonal antibody and terminal deoxynucleotidyl transferase-mediated dUTP-biotin nick-end labeling (TUNEL) kits were purchased from Oncogene Research Products (San Diego, Calif.), and all other primary and secondary antibodies were purchased from Santa Cruz Biotechnology (Santa Cruz, Calif.). Cell viability was analyzed by trypan blue exclusion assay. Cells were trypsinized and an aliquot suspended 1:1 volume with 0.4% trypan blue. Total cell numbers and cell viability counts were assessed using a hemocytometer by light microscopy (Chada et al., 2005).

2. Purification and Treatment with Human IL-24

Full-length mda-7 cDNA was cloned into pCEP4 FLAG vector (Invitrogen, San Diego, Calif.) containing the CMV promoter. The plasmid was transfected into HEK 293 cells, and stable sub-clones were isolated using hygromycin (0.4 μg/ml). Supernatant from 293-IL24 cells was concentrated and purified using affinity chromatography as described (Caudell et al., 2002). Cells were either treated with purified IL-24 protein at 0-30 ng/ml, or co-cultured with 293-IL24 producer cells using a transwell system (Chada et al., 2005; Chada et al., 2004).

3. Gene transfer

Replication-deficient human type 5 adenovirus (Ad5) carrying the mda-7 gene was previously described (Mhashilkar et al., 2001). The mda-7 gene was linked to an internal CMV-IE promoter, followed by an SV40 polyadenylation sequence. The same adenoviral vector containing the sequence for expression of luciferase (Ad-luc) was used as control virus. Cells were plated 1 day before infection. Target cells were infected with adenoviral vectors (Ad-mda7 or Ad-luc) using 625-10,000 viral particles per cell (33-500 pfu/cell). Experimental conditions were optimized to achieve IL-24 protein expression in >70% of cells, based on results of immunohistochemical staining.

4. Immunoblotting

Immunoblotting using various antibodies and standard procedures was performed as described previously (Chada et al., 2005). Primary antibodies tested were: p-STAT3, caspase-3, p-cdc2 (tyr-15), p-cdc25 (ser-216) (Cell Signaling Technologies, Beverly, Mass.), anti-p27 rabbit polyclonal, anti-β-catenin monoclonal, anti-p-Akt anti-Akt, β-actin (Santa Cruz Biotechnology, Santa Cruz Calif.) or anti-IL-24 antibodies (Introgen Therapeutics, Houston, Tex.). Proteins were visualized using enhanced chemiluminescence (Amersham Biosciences). Activation of STAT-3 was determined by immunofluorescence assay using a phospho-STAT3-specific antibody (Chada et al., 2005). Pictures were taken using a fluorescence microscope 1-2 h after staining.

5. FACS Analysis

Cell surface receptor subunits IL-20R1 and IL-22R1 were examined by flow cytometry. Briefly, monolayer cells were detached by adding 0.2% EDTA/PBS, washed once with ice-cold PBS, pelleted and resuspended to 0.1 ml 1% FBS in PBS and incubated with either anti-IL-20R1, anti-IL-22R1 or normal IgG control antibody for 60 min at room temperature. Cells were washed and incubated in FITC-conjugated secondary antibody in 1% FBS in PBS for 30 min on ice. Cell were washed 3 times with 0.1% Tween 20 in PBS, pelleted and resuspended in 500 μl of 1% paraformaldehyde and data were acquire and analyzed. Apoptosis was determined via FACS analysis by either a Annexin V assay which was performed according to the manufacturer's protocol. The cells were analyzed by flow cytometric analysis on a FACScalibur flow cytometer (BD Biosciences, San Jose Calif.). A sample population of 10,000 cells was used for analysis.

6. Immunofluorescence Assay

Cells growing in chamber slides were treated with human IL-24 protein in various concentrations (0-20 ng/ml) for 30 min. Cells were fixed with ethanol:acetic acid (9.5:0.5) and then stained with phospho-Stat3 primary antibody and FITC-labeled secondary antibody. The slides were analyzed using a Nikon fluorescence microscope.

7. Statistical Analysis

The statistical significance of the experimental results was evaluated using the Students t-test. Significance was set at p<0.05.

B. Results

1. Ad-mda7 Vector Induces High Levels of IL-24 Expression and Cell Killing in Breast Cancer Cells

In this study, two well established breast tumor cell line models, MDA-MB231 and MDA-MB453, were evaluated. These breast cancer cells were transduced with Ad-mda7 at various dosages (from 0 to 10,000 virus particles per cell; 0-500 pfu/cell) and after 72 hours, supernatants and cellular lysates were collected and probed by western blotting for IL-24 protein expression. Both cell lines showed high level expression and secretion of IL-24 that increased in correlation with the dose of Ad-mda7. Multiple bands were observed on the blot reflecting processing of IL-24 into its mature glycosylated form. IL-24 expression is the direct result of gene delivery as untreated cells or cells treated with the control vector carrying the luciferase gene (Ad-luc) demonstrated no IL-24 expression.

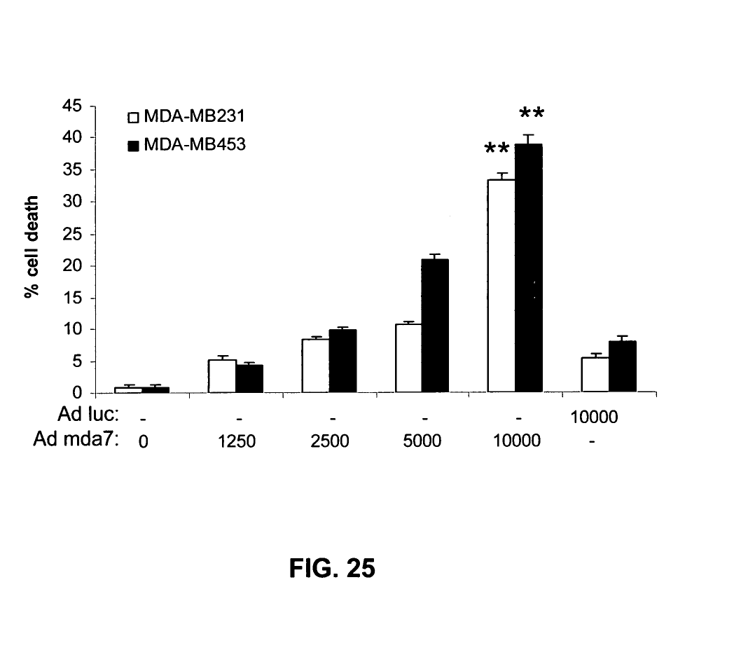

Cell cultures were also monitored for viability by Trypan Blue exclusion analysis after three days. Transduction with a luciferase control vector caused only minor killing compared to untreated cells, whereas Ad-mda7 induced significantly greater (P<0.001) killing in a dose-dependent manner (FIG. 25). Cell killing strongly correlated with the expression of secreted IL-24 mediated by Ad-mda7, with correlation coefficients of 0.98 and 0.93 for MDA-MB231 and MDA-MB453 cells, respectively (Table 5).

TABLE 5
MDA-7/IL-24 expression correlates with cell death

| | |
|---|---|
| MDA-MB231 | MDA-MB453 |

| Ad mda7 | % | IL- | % | IL- |
|---|---|---|---|---|
| (vp/cell) | cell death | 24 signal | cell death | 24 signal |
| 0 | 0.92 | 132 | 0.92 | 87 |
| 1250 | 5.03 | 586 | 4.21 | 381 |
| 2500 | 8.34 | 1279 | 9.71 | 822 |
| 5000 | 10.58 | 3827 | 20.95 | 1783 |
| 10000 | 33.1 | 6104 | 38.75 | 511 |

| | | |
|---|---|---|
| Pearson | 0.93 | 0.98 |
| correlation | | |
| coefficient | | |
| (R) | | |

These data strongly suggest that the observed cell killing effects are the result of increasing levels of IL-24 protein expression.

2. Ad-mda7 Blocks Cell Cycle Progression and Induces Apoptosis in Breast Cancer Cells

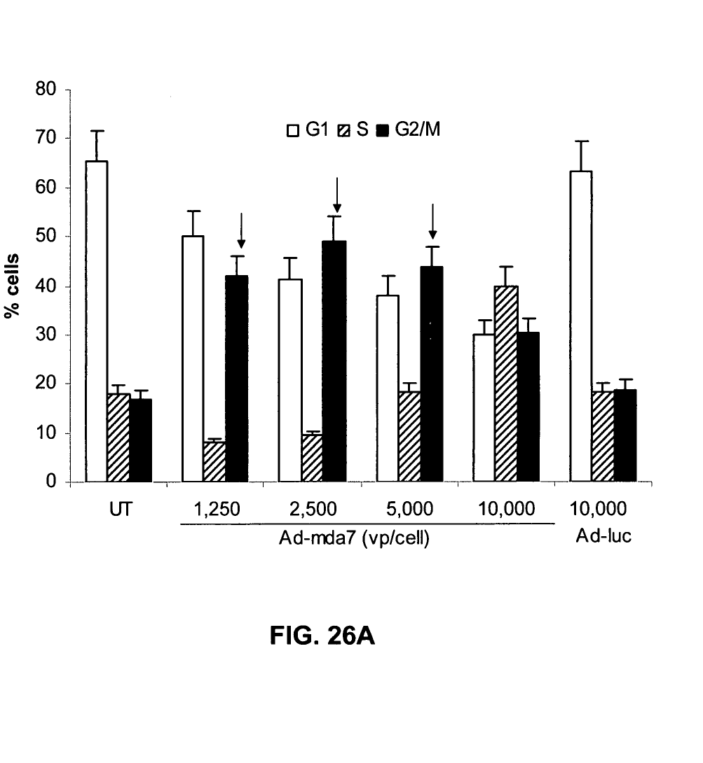

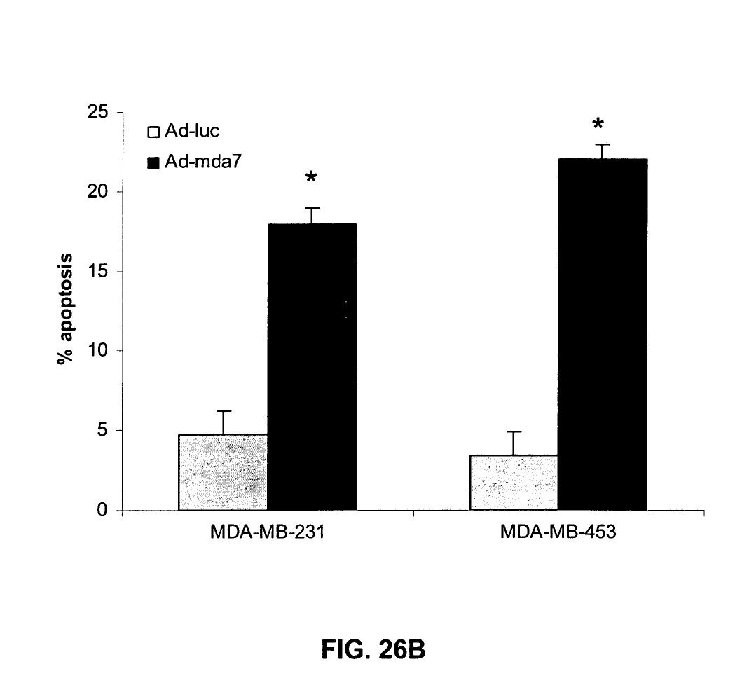

To understand the mechanisms of cell death induced by Ad-mda7 in breast cancer cells, cell cycle analysis by PI staining and flow cytometry was performed. Analysis of cell cycle in Ad-mda7 transduced cells shows a significant increase (p<0.05) in the G₂/M population compared to the untreated control or Ad-luc transduced cells, indicating cell cycle arrest at this phase (FIG. 26A). Further support for Ad-mda7 blocking cell cycle progression was obtained by analysis of cell cycle proteins. Treatment with Ad-mda7 resulted in increased phosphorylation of CDC-25 and an increase in total p27 levels. CDC-25 is a phosphatase involved in cell cycle progression from G2 to M that is inactivated by phosphorylation on Ser216. Inactivation of CDC-25 prevents it from dephosphorylating its downstream targets which would allow cell cycle progression. p27 is another critical cell cycle regulatory protein whose levels increase in quiescent cells. Increased cell cycle block correlated with decreased phosphorylation of CDC-2. Untreated cells, and those transduced with Ad-Luc control vector showed no change in p27 or p-CDC-25 or p-cdc2 levels.

To evaluate the role of Ad-mda7 in activation of programmed cell death pathways, the Annexin V assay was performed to analyze early apoptotic events. Treatment of both MDA-MB231 and MDA-MB453 cells with Ad-mda7 resulted in significant increases in Annexin V positive cells (p<0.01), indicating a higher fraction of apoptosis, as compared to Ad-luc treated controls (FIG. 26B). Western analyses demonstrated dose-dependent cleavage and activation of caspase-3 in breast tumor cell after Ad-mda7 treatment. The P13K survival pathway has been implicated in breast tumorigenesis and chemoresistance; thus the regulation of protein expression in the P13K and Wnt survival signaling pathways in MDA-MB 453 cells was examined (Nicholson and Anderson, 2002; Campbell et al., 2004; Simstein et al., 2003). Western blot analyses showed that increasing levels of MDA-7/IL-24 expression correlated with inhibition of proteins related to cell survival pathways. As IL-24 levels increase within the cell, a concomitant decrease in Akt, p-Akt and β-catenin were observed.

3. Exogenous IL-24 Protein Activates STAT3 and Kills Breast Cancer Cells

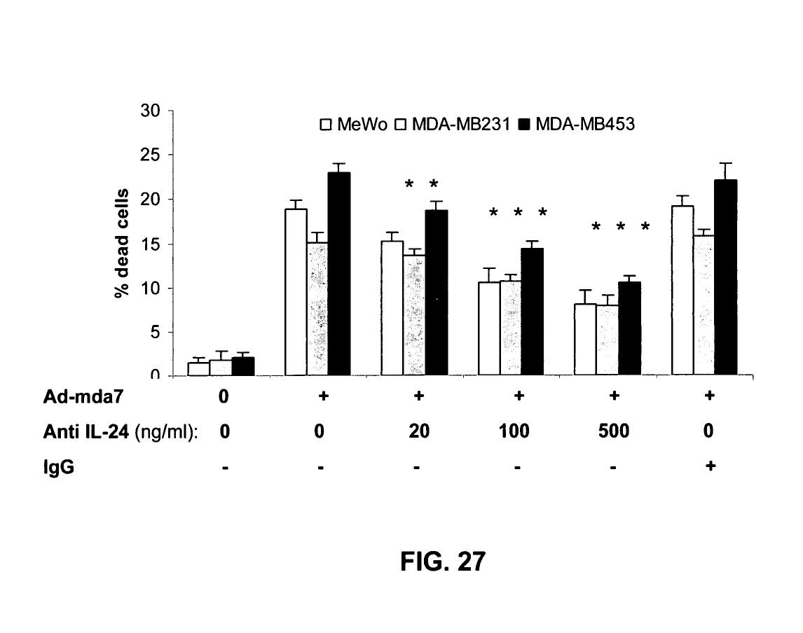

Because IL-24 protein is present in both the supernatant and intracellular compartments of Ad-mda7 transduced cells, the function of intracellular versus extracellular IL-24 in growth inhibition of breast cancer cells was examined. MeWo melanoma cells serve as a positive control cell line since IL-24 has been reported to effectively kill melanoma cells via ligand-receptor engagement (Chada et al., 2004). Increasing concentrations of an anti-MDA7 neutralizing antibody was added to cultures of Ad-mda7 transduced cells. In breast and melanoma cell lines, neutralization of IL-24 significantly decreased cell killing (p<0.01) compared to addition of a nonspecific IgG antibody (FIG. 27), indicating the effect was IL-24 specific. Note that anti-IL-24 was not able to fully abrogate cell killing, suggesting that Ad-mda7 kills breast cancer cells by both intracellular and extracellular mechanisms.

All IL-10 cytokine family members, including MDA-7/IL-24 have been shown to induce the activation of STAT3 in receptor-positive cell lines (Pestka et al., 2004). Therefore IL-24 receptor engagement was evaluated by testing STAT3 phosphorylation and translocation to the nucleus in breast cancer cells. The receptors for IL-24 are heterodimeric cytokine receptors termed type 1 IL-20R (IL-20R1/IL-20R2) and type 2 IL-20R (IL-22R1/IL-20R2). Immunofluorescence microscopy using an antibody directed against phospho-STAT3 shows that both IL-24 and IL-10 were able to activate STAT3 in both breast cancer cell lines.

4. IL-24 Requires Binding to its IL-20 Receptors to Induce Apoptosis

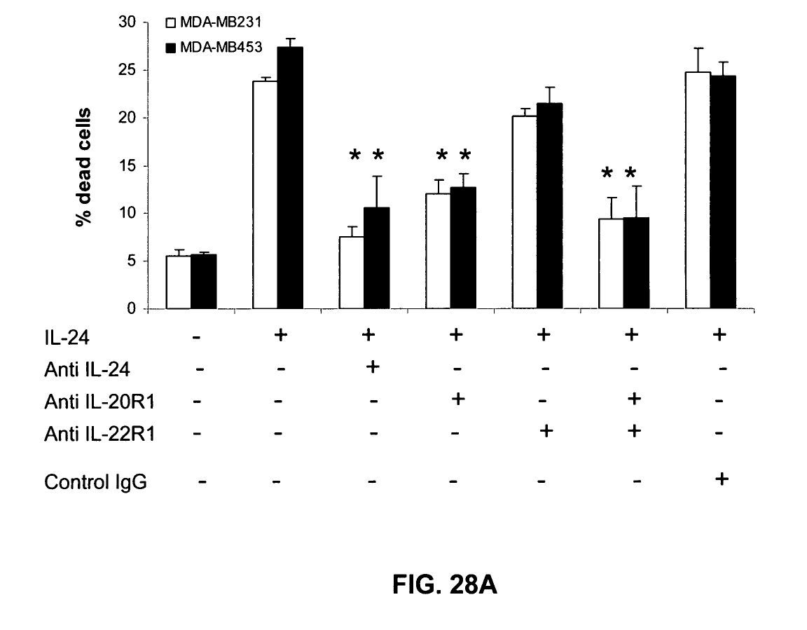

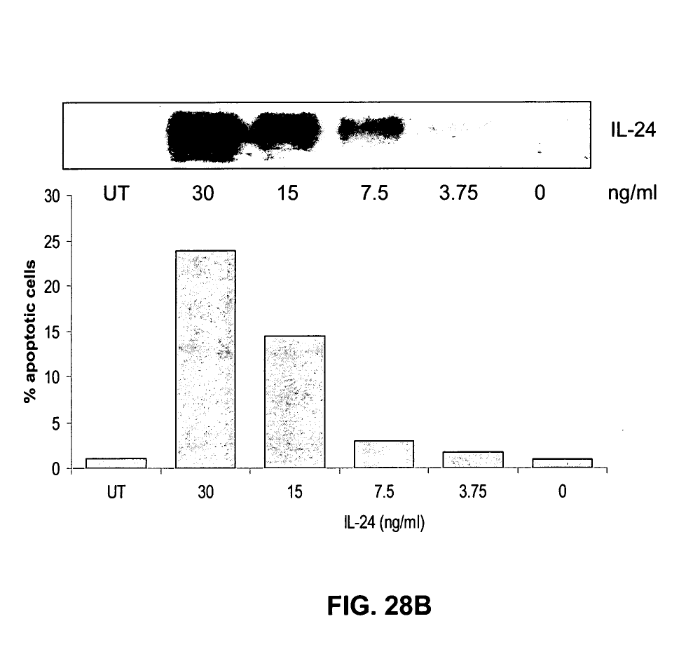

Since IL-24 and IL-10 both bind to related receptors and activate STAT3, but only IL-24 has the ability to kill cells, studies were conducted to identify which receptor mediated cell death by IL-24. MDA-MB453 and MDA-MB231 cells were treated with neutralizing antibodies against IL-24, IL-20R1, or IL-22R1 and then exposed to IL-24 and monitored for cell death using Trypan blue staining. Anti-IL-24 was able to significantly inhibit (p<0.01) IL-24 mediated cell killing by >80%. Anti-IL-22R1 showed a modest reduction (16% for MB231 and 22% for MB453), while anti-IL-20R1 significantly reduced killing (p<0.01) by ≧60% in both cell lines (FIG. 28A). Combining both receptor neutralizing antibodies further reduced killing significantly to levels comparable to controls.

Studies were conducted to examine the cell surface expression of IL-20R1 and IL-22R1 using specific antibodies to these receptors and FACS analysis. The results show that IL-20R1 staining is almost four-fold higher than IL-22R1 staining, suggesting that either IL-20R1 is in greater abundance on the cell surface or that the anti-IL-22R1 antibody has a lower binding affinity than anti-IL-20R1 (Table 6).

TABLE 6
Cell surface IL-20 and IL-22 receptor expression

| Cell | | |
|---|---|---|
| line | IL-20R1+ (%) | IL-22R1+ (%) |
| MeWo | 76 ± 2 | 54 ± 15 |
| A549 | 6 ± 3 | 7 ± 4 |
| MDA | 80 ± 14 | 21 ± 6 |
| 231 | | |
| MDA | 82 ± 16 | 21 ± 3 |
| 453 | | |

The positive control MeWo melanoma cell line expresses high levels of both cell surface IL-20R1 and IL-22R1 receptor subunits, whereas levels of these receptors were very low on A549 cell (lung cancer cells).

5. IL-24, but not Other IL-10 Family Members, Induces Dose-Dependent Apoptosis in Breast Cancer Cells

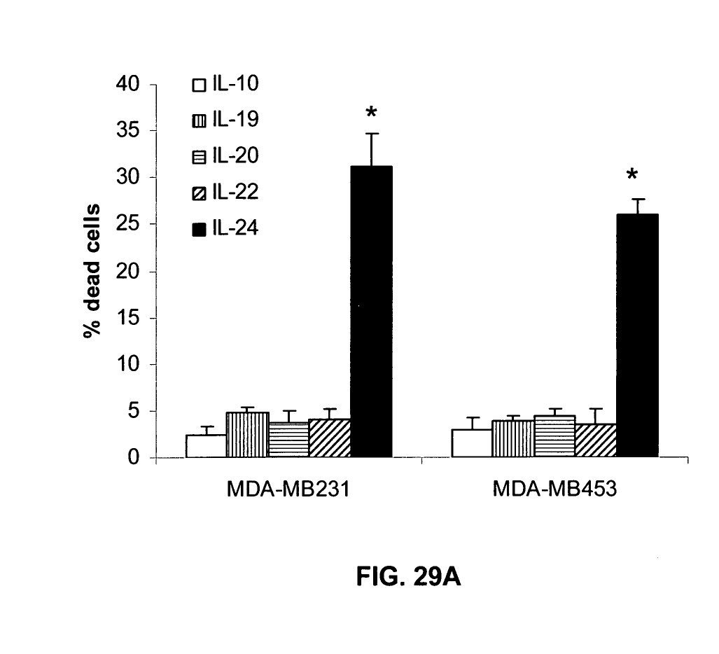

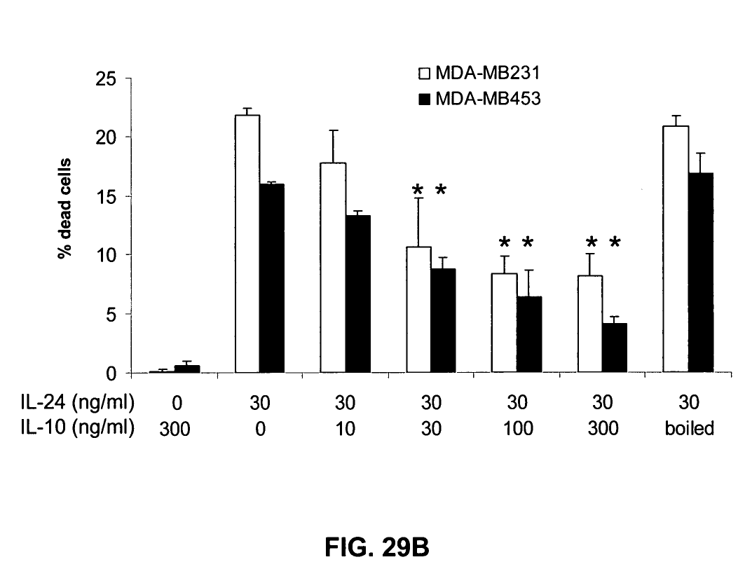

To evaluate the mechanism of cell death mediated by IL-24 protein, the Annexin V assay was used to assess apoptosis in breast tumor cells after exposure to IL-24 protein. Parallel culture supernatants were analyzed for steady-state IL-24 protein levels by Western blotting. Treatment of breast tumor lines with IL-24 protein resulted in induction of significant cell death, with increasing levels of apoptosis directly correlated to the dose of IL-24 (FIG. 28B). This result was not observed with other IL-10 family cytokines. IL-10, IL-19, IL-20 and IL-22 were evaluated for cytotoxicity against breast tumor cells, but none of these induced cell death above background levels (FIG. 29A). In breast cancer cells, although IL-10 and all the other family members can activate STAT3, IL-24 is the only family member with direct cytotoxic properties.

6. IL-10 Antagonizes Killing Mediated by IL-24 Protein

Because previous studies demonstrated that IL-10 blocked the expression of IL-6, interferon-γ, TNF-α and other cytokines induced by IL-24 in PBMCs (Caudell et al., 2002; Wang et al., 2002), studies were conducted to assess whether IL-10 and IL-24 antagonize each other in signal transduction and cell proliferation. To determine whether IL-10 can regulate IL-24 induced growth inhibition, MDA-MB231 and MDA-MB453 cells were both treated with identical amounts of IL-24 protein and increasing amounts of recombinant IL-10. The results show that IL-10 significantly inhibited IL-24-protein-induced killing in a dose-dependent manner (FIG. 29B). IL-10 alone did not stimulate cell proliferation or killing of breast cancer cell lines (FIG. 29A). As an additional control for specificity, IL-10 was boiled to block its function. Boiling of IL-10 abrogated its ability to inhibit killing mediated by IL-24 (FIG. 29B).

### Example 9

### Bevacizumab Enhances Ad-mda7-Mediated Antitumor Activity Against Lung Cancer Cells

A. Materials and Methods

1. Recombinant Adenoviral Vector

Ad-mda7 and Ad-luciferase (luc) vectors were constructed and purified as previously reported (Mhashilkar et al., 2001; Saeki et al., 2000). The transduction efficiencies for the cell lines were determined with an adenoviral vector carrying GFP (Ad-GFP). Transduction efficiency was greater than 80 with each cell line when infected with 3000 vp/cell. Cells were treated with appropriate viral particles.

2. Cell Culture and Reagents.

The non small cell lung cancer cell lines H1299 and A549 were cultured as previously described (Saeki et al., 2000). The HUVECs were purchased from clonetics (Walkersville, Md.) and were grown in endothelial cell basal medium with 5% fetal bovine serum and additional reagents supplied as abullet kit by the manufacturer. Endothelial cells were used at passage 3-8.

3. Cell Proliferation Assay.

To determine non-cytotoxic dose, dose-titration study was done. According to this pilot study, Ad-mda7 at 1000 vp/cell was not cytotoxic to lung tumor cells up to four days while doses at 2000 and 3000 vp/cell were cytotoxic. Based on these results all subsequent assays described below were carried out using Ad-luc or Ad-mda7 at 1000 vp/cell.

Tumor cells (H1299 and A549) were seeded in six-well plates (2×10⁵ cells/well) and treated with an adenoviral vector expressing the luciferase (luc) gene (Ad-luc) or Ad-mda7 (1000 viral particles [vp]/cell). Cells treated with PBS served as a control. Cells were harvested at various time points as listed in figures and subjected to cell viability assay as previously described (Saeki et al., 2000).

For analysis of the effect of conditioned medium from Ad-mda7-treated H1299 on endothelial cell proliferation, human umbilical vein endothelial cells (HUVECs) were seeded in six-well plates (3×10⁵ cells/well). At 24 h after incubation the culture medium was replaced with conditioned medium prepared from tumor cells that were treated with PBS, Ad-luc, or Ad-mda7. HUVECs were incubated for an additional 3 days and later harvested and subjected to cell viability assay (Ramesh et al., 2003; Mhashilkar et al., 2001).

In a separate set of experiments, HUVECs were treated with conditioned medium from Ad-mda7-treated cells that contained recombinant human VEGF₁₆₅ or anti-MDA-7 antibody (10 μg/ml; Introgen Therapeutics, Houston, Tex.) and cell viability was determined as described above.

4. Western Blotting.

Tumor cells treated with PBS, Ad-luc, or Ad-mda7 were harvested at designated time points after treatment. Cell lysates were collected and analyzed by western blotting as previously described (Mhashilkar et al., 2001; Saeki et al., 2000). The following anti-human primary antibodies were used for detection: VEGFR2 (Chemicon, Temecula, Calif.), phosphorylated VEGFR2 (pVEGFR2, Y1214; Biosource, Camarillo, Calif.) VEGF (Santa Cruz Biotechnology, Santa Cruz, Calif.); beta-actin (Sigma Chemical Co., St. Louis, Mo.); AKT, phosphorylated AKT (pAKT), Caspase-3 (Cell Signaling Technology Inc., Beverly, Calif.); and MDA-7 (Introgen Therapeutics).

5. Enzyme-Linked Immunosorbent Assay (ELISA).

Tumor cells were seeded in six-well plates and treated with PBS, Ad-luc, or Ad-mda7 (1000 vp/cell). Conditioned medium were collected at 48 h and 72 h after the treatment and subjected to centrifugation at 13,000 rpm for 15 min to eliminate cell debris. The supernatant was analyzed in triplicates for human VEGF using commercially available ELISA kits (Quantikine human VEGF, R&D Systems). The assays were performed according to the manufacturer's protocol and the VEGF concentrations determined. The VEGF concentration in the conditioned medium of Ad-luc or Ad-mda7 treated cells were expressed as percentage inhibition over the VEGF concentration in the medium of PBS-treated cells. Experiments were performed three times and the results expressed as the average of three separate experiments.

6. Src Kinase Assay.

The Src kinase assay was performed as previously described (Boyd et al., 2004). Briefly cell lysates from PBS-, Ad-luc-, and Ad-mda7-treated tumor cells were prepared in radioimmunoprecipitation assay buffer and reacted with anti-c-Src monoclonal antibody 327 (Oncogene Science Inc., Cambridge, Mass.). Immune complexes were formed with rabbit antimouse IgG and formalin-fixed Pandorbin. The kinase reaction was initiated by adding 10 μCi of [γ-³²P] ATP, 10 mM Mg²⁺, 10 μg of rabbit muscle enolase (Sigma), and 100 μM sodium orthovanadate in 20 mM HEPES. Reactions were terminated with sodium dodecyl sulfate. The radiolabeled protein products were separated in 8% polyacrylamide gel, and subjected to autoradiography. The blots were subjected to semiquantitative analysis using a densitometer and the values represented as percent inhibition over PBS.

7. VEGF Receptor Activation Assay.

HUVECs were seeded in six-well plates (5×10⁵/well) and were starved with growth factor free medium contains 1% FBS overnight. The next day, medium was replaced with conditioned medium collected from tumor cells that were treated with PBS, Ad-luc, Ad-mda7, Ad-mda7 plus Anti-MDA7 antibody, Ad-mda7 plus recombinant human VEGF(rhVEGF). In a different set of experiment, cells were also treated with conditioned medium treated with Avastin, Avastin plus Ad-luc and Avastin plus Ad-mda7. HUVECs to which conditioned medium was not added served as controls. Cells were harvested at 5, 10, and 60 min after addition of conditioned medium, and cell lysates were prepared and analyzed for phosphorylation of VEGF receptor and phosphorylation of AKT, a downstream target of VEGF receptor by western blotting.

8. In Vivo Analysis

To determine if Ad-mda7 plus Bivacizumab combination enhances tumor growth inhibition of tumors in vivo, H1299 tumor cells (5×10⁶) were injected s.c. into lower right flank of athymic BALB/c female nude mice (n=50). The mice were divided into groups and treated as follows when the tumor size reached 50 to 100 mm³: PBS (n=8), Ad-luc (n=8), Ad-mda7 (n=8), Bivacizumab (n=9), Ad-luc plus Bivacizumab (n=8), Ad-mda7 plus Bivacizumab (n=9). The mice were treated with Ad-luc or Ad-mda7 intratumorally (1×10¹⁰ vp/dose) twice a week. Bivacizumab (100 ug/body) was given i.p. twice a week. Animals were weighed weekly and tumor growth was measured thrice a week as described previously (Saeki et al., 2002; Ramesh et al., 2003). At 28 days after the first treatment, all animals were killed via CO₂ inhalation, and tumors were collected for histopathological examination and Western blot analysis. Two different sets of experiments were done.

9. Statistical Analysis.

All experiments were performed three times and Student's t-test and analysis of variance were used to calculate the statistical significance of the experimental results. The significance level was set at P<0.05.

B. Results

1. Ad-mda7 Suppressed VEGF in NSCLC Independent of its Killing Effect

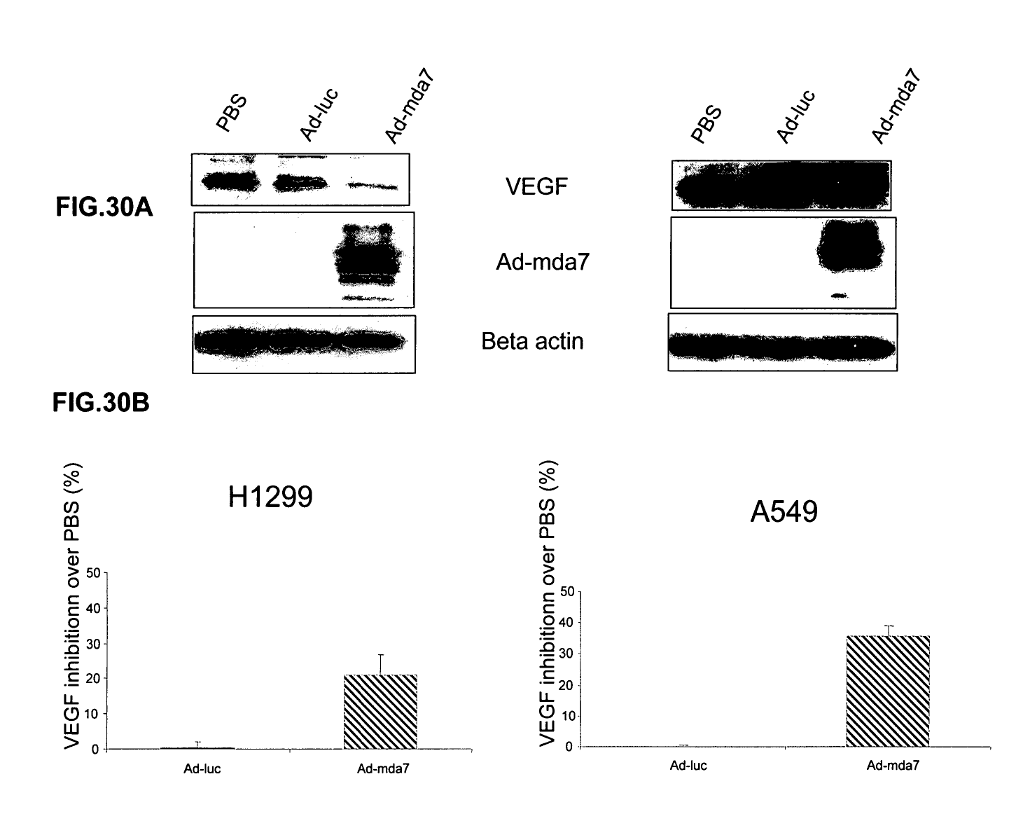

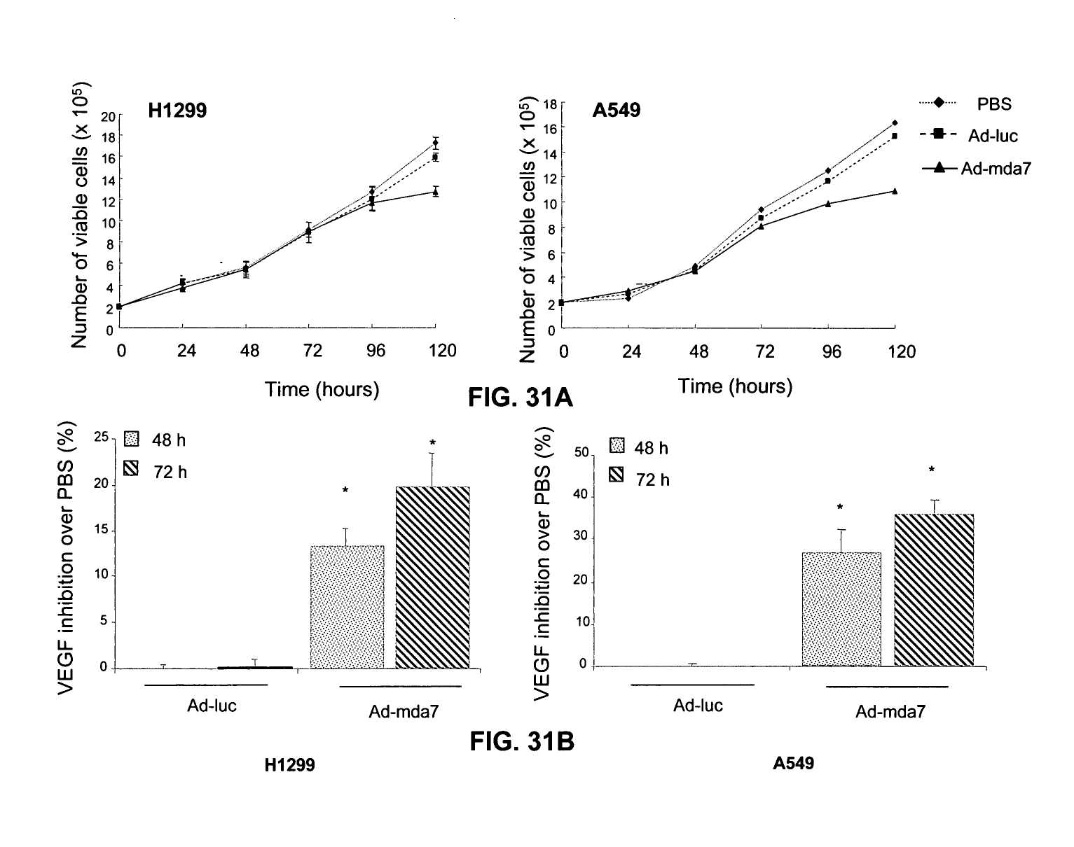

H1299 and A549 were grown in six-well tissue culture plates (2×10⁵) and treated with 1000 VP/cell of Ad-mda7. Cells were treated with PBS and Ad-luc (1000 vp/cell) served as control. Cell extracts and culture supernatant were collected at 48 hours and 72 hours after the treatment. Ad-mda7 markedly decreased VEGF expression in both NSCLC (FIG. 30A) comparing with PBS and Ad-luc. And MDA-7 protein expression was observed to be time dependent. Cell viability assay revealed that 1000 vp/cell of Ad-mda7 did not show any killing effect on both lung cancer cell lines up to 72 hours after the treatment (FIG. 31A-B). Analysis of VEGF in the culture supernatant showed that Ad-mda7 significantly suppressed VEGF in both NSCLC as compared to Ad-luc (FIG. 30B, FIG. 31B). These data suggest that Ad-mda7 inhibits VEGF expression in NSCLC independent of its toxic effect against tumor cells.

2. Ad-mda7 Downregulated Src Activation in NSCLC.

Previouly several studies have shown that c-Src, a non receptor kinase plays a role in regulating VEGF expression and high expression of Src has been reported to be associated with increased VEGF expression (Inoue et al., 2005; Irby and Yeatman, 2000; Bromann et al., 2004). Based upon these reports we examined whether Ad-mda7-mediated VEGF inhibition involved c-Src in NSCLC by Src kinase assay.

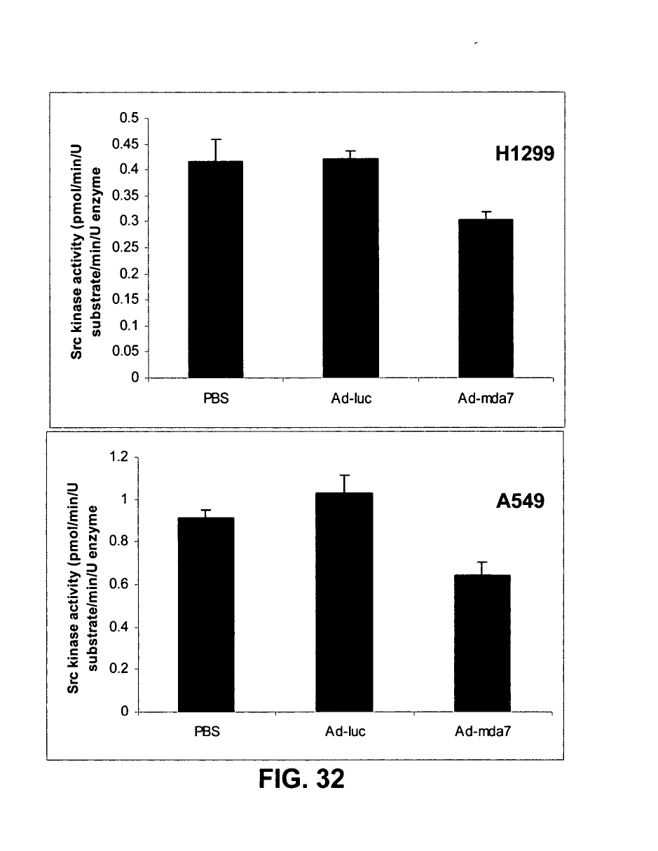

H1299 and A549 cells were seeded in six-well plates and lysates were collected 48 hours after the treatment with PBS, Ad-luc and Ad-mda7. Src activity was analyzed using kinase assay kit. Ad-mda7 significantly decreased Src activity in both NSCLC (FIG. 32). This data suggests that inhibition of Src activity by Ad-mda7 causes downregulation of VEGF in lung cancer cells.

3. MDA-7-mediated VEGF Inhibition in Lung Tumor Cells Affects Endothelial Cell Proliferation and VEGF Receptor Signaling

VEGF has been shown to be a key growth factor for endothelial cell proliferation and survival (Gerber et al., 1998). Therefore inhibition of VEGF should offer an endothelial cell death. To investigate whether decreased VEGF production by tumor cells affected endothelial cell proliferation and signaling, a cell viability assay and Western blot analysis for VEGF receptor signaling on HUVEC was performed.

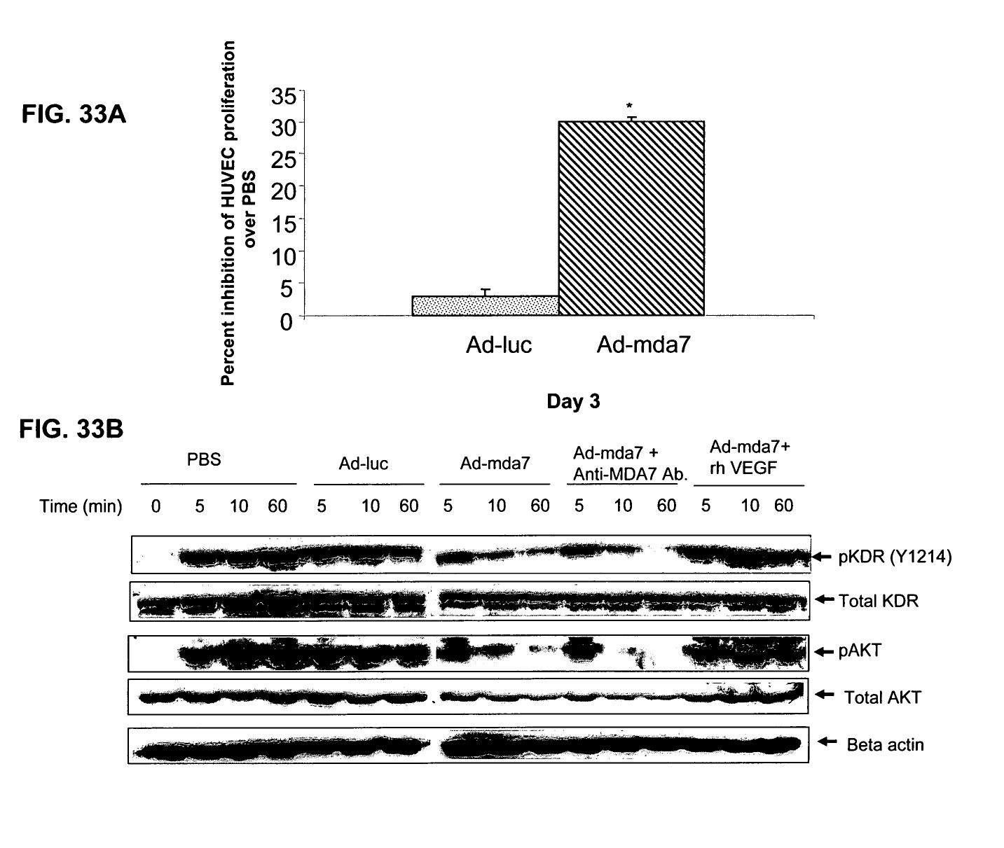

Treatment of HUVECs with conditioned medium collected from Ad-mda7-treated tumor cells showed significant inhibition of cell proliferation (FIG. 33A) as compared to treatment with conditioned medium from Ad-luc-treated tumor cells when data was denoted by percent inhibition over PBS group (FIG. 33A). To further evaluate whether the inhibition of HUVEC proliferation was due to reduced VEGF, serum-starved HUVECs were treated as described above and analyzed for VEGF receptor signaling. In addition, excess volume of anti-MDA7 neutralizing antibody was added to exclude the possibility that secreted form of MDA-7 protein might affect HUVEC proliferaton. VEGFR2 and AKT, downstream target for VEGFR2 signaling pathway, were phosphorylated when HUVECs were treated with conditioned medium from PBS and Ad-luc treated tumor cells. Wherever VEGFR2 and AKT activation were not observed in HUVECs treated with conditioned medium from Ad-mda7-treated cells in the presence or absence of anti-MDA7 neutralizing antibody. Addition of rhVEGF₁₆₅ to conditioned medium from Ad-mda7-treated cells restored VEGFR2 and AKT activation (FIG. 33B).

4. Ad-mda7 did not Show any Killing Effect on HUVECs and Avastin Inhibited HUVEC Cell Proliferation but did not Affect H1299 Cell Proliferation

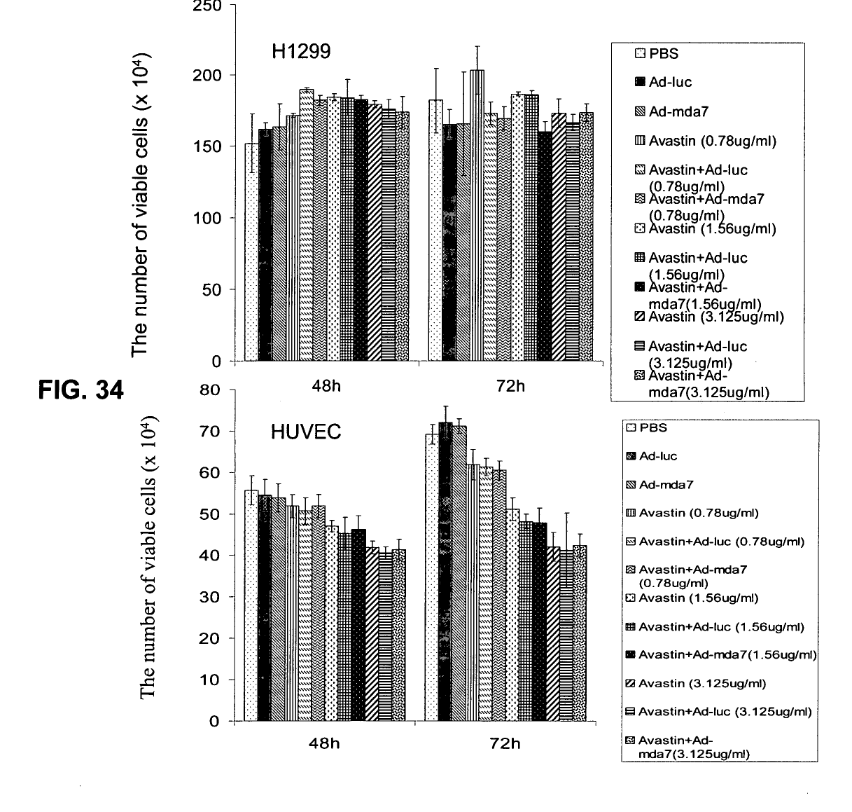

Studies were conducted to investigate whether Ad-mda7 and Bivacizumab combination is effective against lung cancer. To evaluate the toxicity of both Ad-mda7 and Bivacozumab against tumor cells and endothelial cells, a cell viability assay was performed. H1299 cells (2×10⁵/well) and HUVECs (3×10⁵/well) were seeded in six-well plates. The next day, cells were treated with PBS, Ad-luc (1000 vp/cell), Ad-mda7 (1000 vp/cell) and Bivacizumab (0.78 ug/ml, 1.56 ug/ml, 3,125 ug/ml), Ad-luc plus Bivacizumab and Ad-mda7 plus bivacizumab respectively. At 48 hours and 73 hours after the treatment, cells were trypsinized and analyzed for trypan blue cell exclusion assay. Consistent with previous data, 1000 vp/cell of Ad-mda7 did not show any toxicity both on H1299 and HUVECs. Whereas Bivacizumab inhibited HUVEC viability, however, Bevacizumab did not show harmful effect on H1299 (FIG. 34).

5. VEGF Inhibition in Lung Tumor Cells by MDA-7 and Avastin Affects Endothelial Cell Proliferation

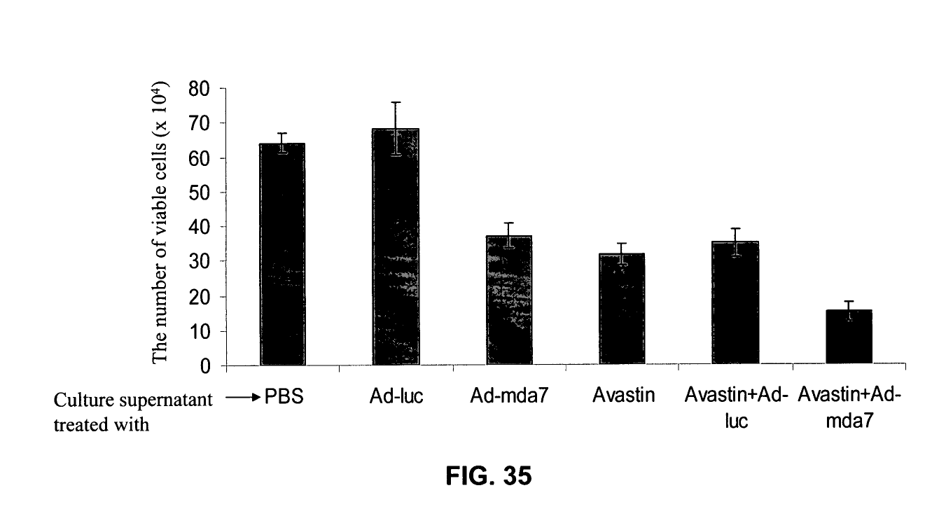

Treatment of HUVECs with conditioned medium collected from Ad-mda7-treated tumor cells showed significant inhibition of cell proliferation (P=<0.05; FIG. 34) as compared to treatment with conditioned medium from Ad-luc-treated tumor cells when data was denoted by percent inhibition over PBS group (FIG. 35).

6. Blockade of VEGFR2 Signaling on HUVEC was Significantly Enhanced when Ad-mda7 was Combined with Bivacizumab

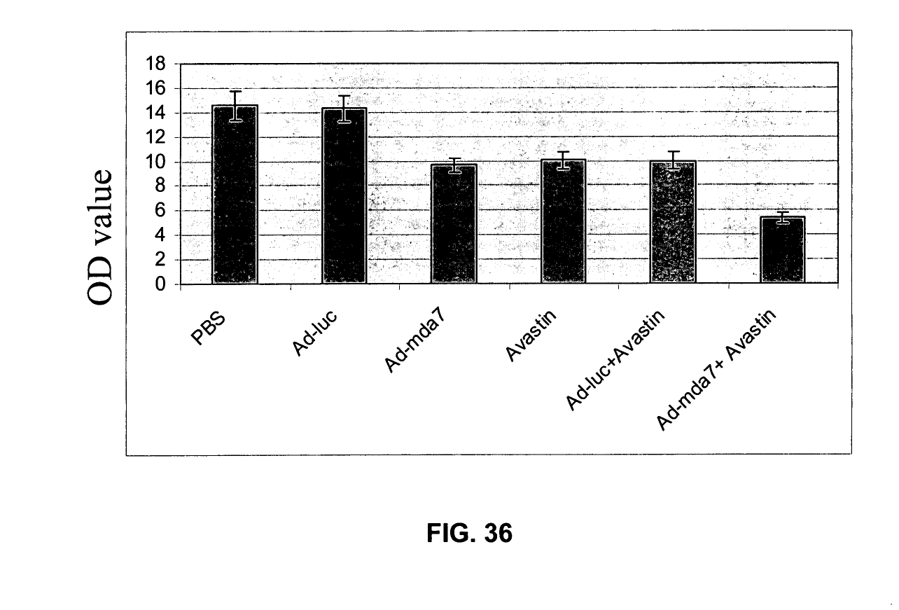

To evaluate whether Ad-mda7 with Bivacizumab enhances suppressive effect of VEGFR2 signaling pathway on HUVECs, the same set of experiment as mentioned above were conducted, except that Ad-mda7 was combined with Bivacizumab. Again, conditioned medium from PBS and Ad-luc treated tumor cells activated VEGFR2 signaling. Less VEGFR2 and AKT activation were observed in HUVECs treated with conditioned medium from Ad-mda7-treated cells and Bevacizumab-treated cells. Furthermore, the inhibitory effect of VEGFR2 and AKT activation on HUVEC by Bivacizumab-treated supernatant were similar to that of Ad-mda7-treated, and Ad-luc plus Bivacizumab-treated supernatant. Activation of VEGFR2 signaling was markedly reduced when HUVECs were treated by culture supernatant treated with Ad-mda7 plus Bivacizumab (FIG. 36).

7. Ad-mda7 plus Bivacizumab Combination Enhances Tumor Growth Suppression In Vivo

To evaluate whether Ad-mda7 plus Bivacizumab combination treatment enhances tumor growth suppression, in vivo experiments were conducted using a xenograft model. Mice treated with Ad-mda7 plus Bevacizumab suppressed tumor growth significantly compared with mice treated with PBS, Ad-luc, Bivacizumab or Ad-luc plus Bevacizumab. Moreover, no adverse effect associated with each agent or combination such as body weight loss, morbidity ro death was observed, suggesting that all treatments were tolerable.

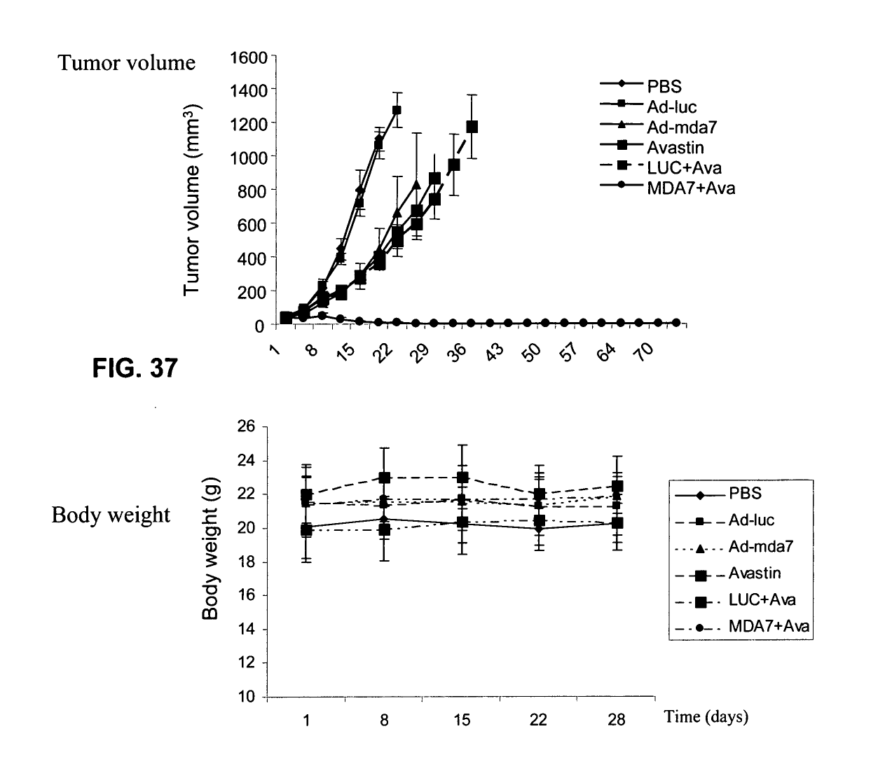

To examine the molecular mechanism of tumor inhibition, animals were euthanized 24 hours after the last treatment and tumor samples collcected and snap frozen or fixed in formalin. Total protein was extracted from snap frozen tumor tissue and were analyzed for VEGF, MDA-7, and Caspase-3 by Western blot analysis. The mda-7 gene were successfully transfected into tumor cells. VEGF in tumor sample was suppressed by Ad-mda7 treatment, while PBS and Ad-luc did not inhibit (FIG. 37). VEGF expression were significantly reduced when tumor was treated by Ad-mda7 plus Bivacizumab. Moreover, cleaved caspase-3 was observed in the tumor treated with Ad-mda7. Interestingly, more caspase-3 cleavage was observed when the tumor was treated by Ad-mda7 plus Bivacizumab.

Immunohistochemical analysis of tumor tissues for MDA-7, VEGF, CD31 and TUNEL showed tumors from mice that were treated with AD-mda7 and Bevacizumab showed a significant reduction in VEGF, CD31 and increased TUNEL positive staining compared to all other treatment groups. Additionally, the observed effects correlated with MDA-7 protein expression.

In summary, Ad-mda7 suppresses VEGF in NSCLC through inhibition of Src kinase activity. Blockade of VEGFR2 signaling on HUVEC was significantly enhanced when Ad-mda7 was combined with Bivacizumab. MDA-7-mediated VEGF inhibition in lung tumor cells affects endothelial cell prpoliferation and VEGF receptor signaling. Ad-mda7 plus Bivacizumab combination enhances tumor growth suppression in vivo. Ad-mda7 plus Bivacizumab combination enhances the inhibitory effect of VEGF and tumor apoptosis in vivo.

### Example 10

### Ad-mda7 Plus TNF-Alpha Treatment Enhances Prostate Tumor Cell Killing

A. Materials and Methods

1. Recombinant Adenoviral Vector

Ad-mda7 and Ad-luciferase (luc) vectors were constructed, purified and supplied by Introgen Therapeutics, Inc, Houston, Tex.

2. Transduction Efficiency

The transduction efficiencies for the prostate cancer cell line LNCaP was determined with an adenoviral vector carrying GFP (Ad-GFP). Cells were seeded in six-well plates and treated with different doses of Ad-GFP (100, 300, 600 and 1200 vp/cell). Ad-GFP treated cells were subsequently not treated or treated with recombinant human TNF-alpha protein (10 ng/ml). Cells were harvested at 24 h after TNF-alpha treatment by trypsinization, washed three times with PBS and resuspended in PBS and subjected to FACS analysis. Cells treated with PBS served as controls.

3. Cell Culture and Reagents.

The prostate cancer cell lines LNCaP and DU145 were cultured as recommended by ATCC.

4. Cell Proliferation Assay.

Tumor cells (LNCaP) were seeded in six-well (5×10⁴) or 96-well plates (2×10³ cells/well) and treated with an adenoviral vector expressing the luciferase (luc) gene (Ad-luc) or Ad-mda7 (1500-2000 viral particles [vp]/cell) alone or in combination with TNF-alpha (5 ng/ml). Cells treated with PBS served as a control. Cells were subjected to cell viability assay at 48 and 72 h after treatment using the XTT method as previously described.

5. Western Blotting

Tumor cells treated with Ad-mda7 (1000-2000 vp/cell), Ad-mda7 plus TNF-alpha (10-20 ng/ml) or Ad-mda7 plus Anti-TNF-alpha antibody (0.5-1 ug/ml) were harvested at 24 h after treatment. Cell lysates were collected and analyzed for MDA-7 protein expression by western blotting.

6. Cell Cycle

Tumor cells (LNCaP) were seeded in six-well plates and treated with Ad-mda7 or Ad-luc (1500 vp/cell), Ad-mda7 plus TNF-alpha (10 ng/ml), Ad-mda7 plus anti-TNF antibody (1 ug/ml), Ad-luc plus TNF-alpha or Ad-luc plus anti-TNF antibody. Cells were harvested at 48 h after treatment and analyzed for the number of cells in the SubG0/G1 phase by flow cytometry. Cells treated with PBS served as controls.

B. Results

1. Ad-mda7 Plus TNF-Alpha Treatment Enhances Prostate Tumor Cell Killing

Prostate tumor (LNCaP) tumor cells were treated with PBS, TNF-alpha, Ad-Luc, Ad-mda7, Ad-luc plus TNF, or Ad-mda7 plus TNF. Viral treatment was at 2000 vp/cell and TNF treatment at 5 ng/ml. At 48 h after treatment cells were visualized under a bright-field microscope. Cells treated with Ad-mda7 plus TNF showed significant inhibition of cell proliferation compared to other treatment groups.

2. Ad-mda7 plus TNF-alpha Treatment Inhibits Tumor Cell Proliferation

Prostate tumor (LNCaP) tumor cells were treated with PBS, TNF-alpha, Ad-Luc, Ad-mda7, Ad-luc plus TNF, or Ad-mda7 plus TNF. Viral treatment was at 1500 vp/cell and TNF treatment at 5 ng/ml. At 48 h and 72 h after treatment cells were subjected to XTT assay to determine cell viability. Cells treated with Ad-mda7 plus TNF showed significant growth inhibition compared to other treatment groups (FIG. 37). The inhibitory effect was synergistic.

3. Ad-mda7 Plus TNF-alpha Treatment Increases Exogenous MDA-7 Protein Expression

Prostate tumor cells (LNCaP and DU145) seeded in six-well plates were treated with Ad-mda7 (1000-2000 vp/cell), Ad-mda7 plus TNF-alpha (10-20 ng/ml), and Ad-mda7 plus anti-TNF antibody (0.5-1 ug/ml). Cells were harvested at 24 h after treatment and analyzed for MDA-7 protein expression by western blotting. LNCaP cells treated with Ad-mda7 (2000 vp/cell) showed MDA-7 expression. However, cells treated with Ad-mda7 plus TNF-alpha (20 ng/ml) showed a marked increase in exogenous MDA-7 protein expression that was inhibited in the presence of anti-TNF antibody (0.5 ug/ml). LNCaP and DU145 cells treated with Ad-mda7 (1500 vp/cell) showed MDA-7 expression. However, cells treated with Ad-mda7 plus TNF-alpha (10 ng/ml) showed a marked increase in exogenous MDA-7 protein expression that was abrogated in the presence of anti-TNF antibody (1.0 ug/ml).

4. TNF-Alpha Increases the Transduction Efficiency

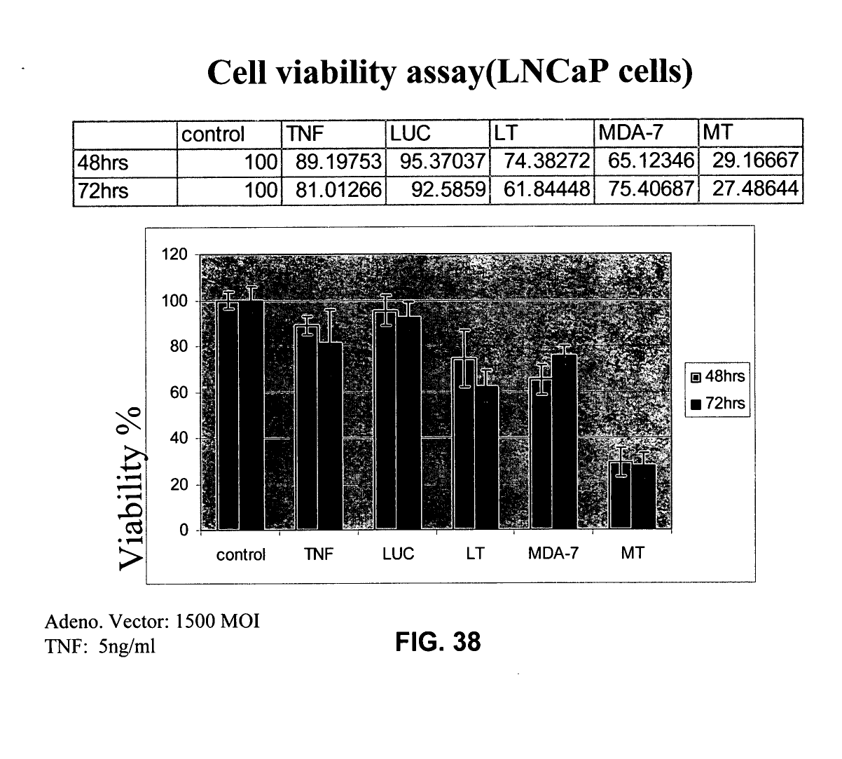

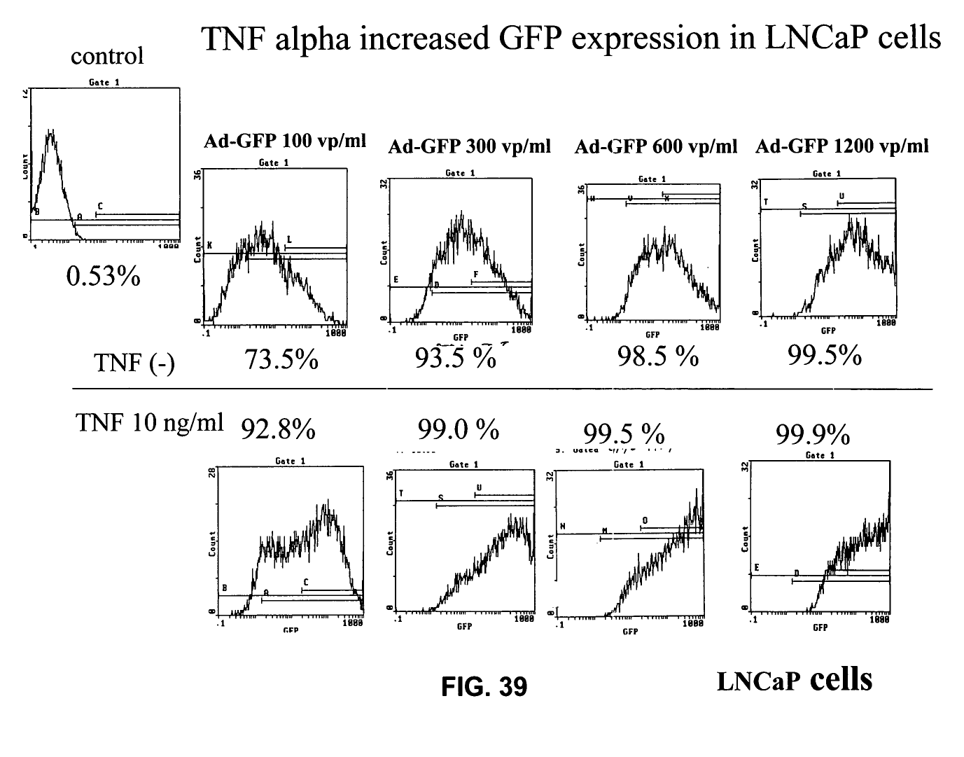

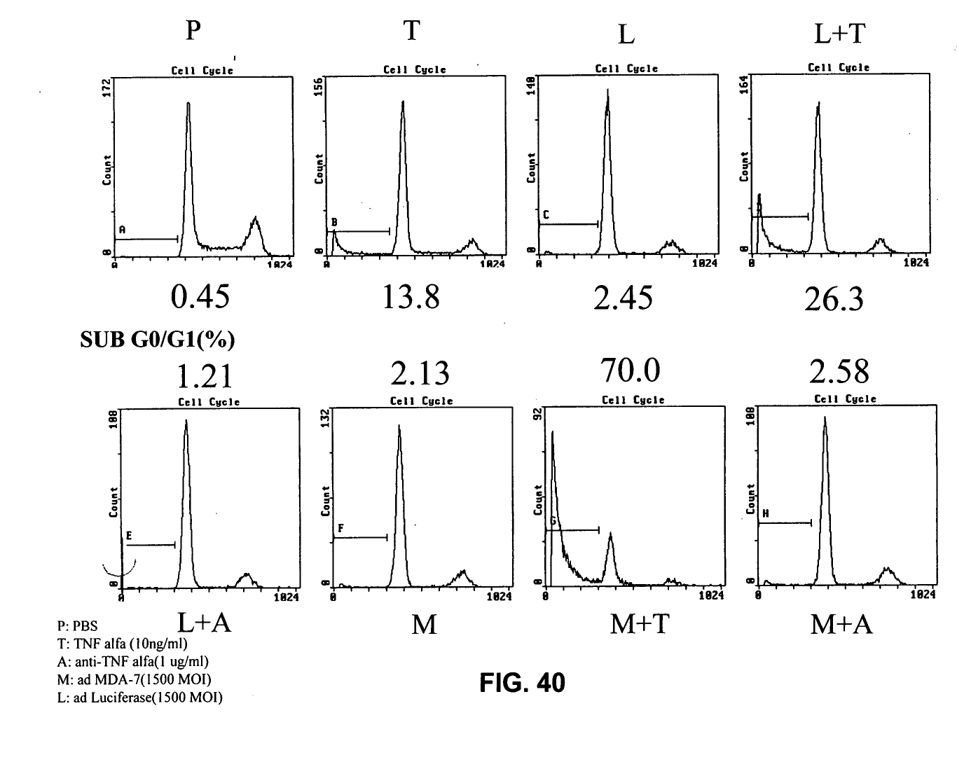

Tumor (LNCaP) cells were treated with Ad-GFP at 100, 300, 600 and 1200 vp/cell in the presence or absence of TNF-alpha (10 ng/ml). Cells receiving no treatment served as control. At 24 h after TNF-alpha treatment cells were harvested, washed with PBS three times, resuspended in 500 ul PBS and subjected to FACS analysis. Cells treated with Ad-GFP alone showed a dose-dependent increase in transduction efficiency starting from 73.5% for 100 vp/cell of Ad-GFP (FIG. 38). However, in the presence of TNF-alpha, the transduction efficiency was increased and was observed to be 92.8% for 100 vp/cell of Ad-GFP. The increase in transduction appeared to be saturated from 300 vp/cell of Ad-GFP in the presence of TNF-alpha.

5. Ad-mda7 Plus TNF-Alpha Treatment Results in Increased Number of Cells in SubG0/G1 Phase

Tumor (LNCaP) cells were treated with PBS, TNF-alpha (10 ng/ml), Ad-Luc (1500 vp/cell), Ad-mda7 (1500 vp/cell), Ad-luc plus TNF, Ad-mda7 plus TNF, Ad-luc plus anti-TNF antibody (1 ug/ml) or Ad-mda7 plus anti-TNF antibody. At 48 h after treatment cells were harvested, washed three times with PBS, resuspended in 500 ul of PBS containing propidium iodide (0.5 ug/ml). Cells were subjected to FACS analysis. A significant number of cells treated with Ad-mda7 plus TNF was observed in the SubG0/G1 phase (70%) indicated apoptotic cells compared to other treatment groups that ranged from 0.45% to 26.3%.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,262,017
U.S. Pat. No. 4,554,101
U.S. Pat. No. 4,797,368
U.S. Pat. No. 4,870,287
U.S. Pat. No. 5,139,941
U.S. Pat. No. 5,179,122
U.S. Pat. No. 5,187,260
U.S. Pat. No. 5,399,363
U.S. Pat. No. 5,466,468
U.S. Pat. No. 5,543,158
U.S. Pat. No. 5,641,515
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,225
U.S. Pat. No. 5,846,233
U.S. Pat. No. 5,846,945
U.S. Pat. No. 6,858,227
U.S. application Ser. No. 09/575,473
U.S. application Ser. No. 09/615,154
U.S. application Ser. No. 10/017,472
U.S. application Ser. No. 10/378,590
U.S. application Ser. No. 10/391,068
U.S. application Ser. No. 10/791,692
U.S. application Ser. No. 10/791,692
U.S. application Ser. No. 60/661,680
U.S. Pub. No. 20030147966
U.S. Pub. No. 20030223938
U.S. Pub. No. 20050143336
Agnew et al., J. Chromatogr. B Biomed. Sci. Appl., 755(1-2):237-243, 2001.
Aksentijevich et al., Hum. Gene Ther., 7(9):1111-1122, 1996.
Alberts et al., J. Cell. Biochem. Supp., (22):18-23, 1995.
Amat et al., Brit. J. Cancer, 88:1339-1345, 2003.
Angel et al., Cell, 49:729, 1987b.
Angel et al., Mol. Cell. Biol., 7:2256, 1987a.
Arap et al., Cancer Res., 55(6):1351-1354, 1995.
Atchison and Perry, Cell, 46:253, 1986.
Atchison and Perry, Cell, 48:121, 1987.
Austin-Ward and Villaseca, Revista Medica de Chile, 126(7):838-845, 1998.
Ausubel et al., In: Current Protocols in Molecular Biology, John, Wiley & Sons, Inc, New York, 1996.
Bajorin et al., J. Clin. Oncol., 6(5):786-792, 1988.
Bakhshi et al., Cell, 41(3):899-906, 1985.
Bakin et al., J. Biol. Chem., 275:36803-36810, 2000.
Banerji et al., Cell, 27(2 Pt 1):299-308, 1981.
Banerji et al., Cell, 33(3):729-740, 1983.
Barber, Death Differ., 8:113-126, 2001.
Basu et al., Mol. Cancer Res., 2:632-642, 2004.
Bateman and Uccellini, J. Pharm. Pharmacol., 36(7):461-464, 1984.
Bedi et al., Cancer Res., 55(9):1811-1816, 1995.
Benoit et al., Biochem. Cell Biol., 82(6):719-727, 2004.
Benoit et al., Oncogene, 23:1631-1635, 2004.
Berkhout et al., Cell, 59:273-282, 1989.
Birringer et al., Br. J. Cancer, 88(12):1948-55, 2003.
Blanar et al., EMBO J., 8:1139, 1989.
Bodine and Ley, EMBO J., 6:2997, 1987.
Bonnadona and Karnofsky, J. Clin. Oncol., 7:1380-1397, 1989.
Bonvini et al., Cancer Res., 61:1671-1677, 2001.
Boshart et al., Cell, 41:521, 1985.
Bosze et al., EMBO J., 5(7):1615-1623, 1986.
Boyd et al., Clin. Cancer Res., 10:1545-1555, 2004.
Braddock et al., Cell, 58:269, 1989.
Bromann et al., Oncogene, 23:7957-7968, 2004.
Bukowski et al., Clin. Cancer Res., 4(10):2337-2347, 1998.
Bulla and Siddiqui, J. Virol., 62:1437, 1986.
Burow et al., Biochem. Biophys. Res. Commun., 271:342-345, 2000.
Caldas et al., Nat. Genet., 8(1):27-32, 1994.
Campbell and Villarreal, Mol. Cell. Biol., 8:1993, 1988.
Campbell et al., Cancer Res., 64(21):7678-7681, 2004.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Campere and Tilghman, Genes and Dev., 3:537, 1989.
Campo et al., Nature, 303:77, 1983.
Caudell et al., J. Immunol., 168(12):6041-6046, 2002.
Celander et al., J. Virology, 62:1314, 1988.
Chada et al., Int. Immunopharmacol., 4(5):649-667, 2004.
Chada et al., Int. Immunopharmacol., 4:649-667, 2004.
Chada et al., Mol. Ther., 10(6):1085-1095, 2004.
Chada et al., Mol. Ther., 11(5):724-733, 2005.
Chada et al., Mol. Ther., 7:S446, 2003.
Chandler et al., Cell, 33:489, 1983.
Chang et al., Mol. Cell. Biol., 9:2153, 1989.
Chatterjee et al., Proc. Natl. Acad. Sci. USA, 86:9114, 1989.
Cheng et al., Cancer Res., 54(21):5547-5551, 1994.
Choi et al., Cell, 53:519, 1988.
Christodoulides et al., Microbiology, 144(Pt 11):3027-3037, 1998.
Clark et al., Hum. Gene Ther., 6(10):1329-1341, 1995.
Cleary and Sklar, Proc. Natl. Acad. Sci. USA, (21):7439-7443, 1985.
Cleary et al., J. Exp. Med., 164(1):315-320, 1986.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Craven et al., Surg. Oncol., 12(1):39-49, 2003.
Cripe et al., EMBO J., 6:3745, 1987.
Culotta and Hamer, Mol. Cell. Biol., 9:1376, 1989.
Dandolo et al., J. Virology, 47:55-64, 1983.
Davidson et al., J. Immunother., 21(5):389-398, 1998.
De Villiers et al., Nature, 312(5991):242-246, 1984.
Denkert et al., Clin. Breast Cancer, 4(6):428-433, 2004.
Deschamps et al., Science, 230:1174-1177, 1985.
Dietary Reference Intakes for Vitamin C, Vitamin E, Selenium, and Carotenoids from the Panel on Dietary Antioxidants and Related Compounds, Institutue of Medicine, Food and Nutrition Board, National Academy Press Washington, D.C., 2000.
Dillman, Cancer Biother. Radiopharm., 14(1):5-10, 1999.
Donze et al., EMBO J., 20:3771-3780, 2001.
DuBois et al., Gastroenterology, (25):773-791, 1996.
Earnest et al., J. Cell Biochem. Suppl., 161:156-166, 1992.
Edbrooke et al., Mol. Cell. Biol., 9:1908, 1989.
Edlund et al., Science, 230:912-916, 1985.
Ekmekcioglu et al., Int. J. Cancer, 94(1):54-59, 2001.
El-Rayes et al., Mol. Cancer Ther., 3:1421-1426, 2004.
Enmon et al., Cancer Res., 63:8393-8399, 2003.
Fariss et al., Cancer Res., 54:3346-3351, 1994.
Felgner et al., Proc. Natl. Acad. Sci. USA, 84(21):7413-7417, 1987.
Feng and Holland, Nature, 334:6178, 1988.
Firak and Subramanian, Mol. Cell. Biol., 6:3667, 1986.
Fisher et al., J. Natl. Cancer Inst., 89(22):1673-1682, 1997.
Flotte et al., Am. J. Respir. Cell Mol Biol., 7(3):349-356, 1992.
Flotte et al., Proc. Natl. Acad. Sci. USA, 90(22):10613-10617, 1993.
Flotte, et al., Gene Ther., 2(1):29-37, 1995.
Foecking and Hofstetter, Gene, 45(1):101-105, 1986.
Fraley et al., Proc. Natl. Acad. Sci. USA, 76:3348-3352, 1979.
Freifelder, In: Physical Biochemistry Applications to Biochemistry and Molecular Biology, 2nd Ed. Wm. Freeman and Co., NY, 1982.
Freshney, In: Animal Cell Culture, A Practical Approach, 2^{nd} Ed., Oxford Press, UK, 1992.
Fry, Breast Cancer Res., 3:304-312, 2001.
Fujita et al., Cell, 49:357, 1987.
Gabizon et al., Cancer Res., 50(19):6371-6378, 1990.
Gaensler et al., Nat. Biotechnol., 17:1188-1192, 1999.
Gann et al., J. Natl. Cancer Inst., 85:1220-1224, 1993.
Garner et al., Cochrane Database Syst Rev., (4):CD003831, 2002.
Gerber et al., J. Biol. Chem., 273:30336-30343, 1998. Celander and Haseltine, J. Virology, 61:269, 1987.
Gewies et al., Cancer Res., 60:2163-2168, 2000.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Giovannucci et al., Ann. Intern. Med., 121:241-246, 1994.
Gloss et al., EMBO J., 6:3735, 1987.
Godbout et al., Mol. Cell. Biol., 8:1169, 1988.
Goding, In: Monoclonal Antibodies: Principles and Practice, 2d ed., Academic Press, Orlando, Fla., pp 60-61, 71-74, 1986.
Goodbourn and Maniatis, Proc. Natl. Acad. Sci. USA, 85:1447, 1988.
Goodbourn et al., Cell, 45:601, 1986.
Greenberg et al., J. Natl. Cancer Inst., 85:912-916, 1993.
Greene et al., Immunology Today, 10:272, 1989
Grosschedl and Baltimore, Cell, 41:885, 1985.
Hanibuchi et al., Int. J. Cancer, 78(4):480-485, 1998.
Hanif et al., Biochemical Pharmacology, (52):237-245, 1996.
Harlow and Lane, In: Antibodies: A Laboratory Manual, Cold Spring Harbor Laboratory Press, NY, 1988.
Haslinger and Karin, Proc. Natl. Acad. Sci. USA, 82:8572, 1985.
Hauber and Cullen, J. Virology, 62:673, 1988.
Hellstrand et al., Acta Oncol., 37(4):347-353, 1998.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hinz and Brune, Wien Klin Wochenschr., 111 (3): 103-12, 1999.
Hirochika et al, J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Howe et al., Cancer Res., 62:5405-5407, 2002.
Howe et al., Endocr. Relat. Cancer, 8:97-114, 2001.
Hsu et al., J. Biol. Chem., 275:11397-11403, 2000.
Hu et al., J. Antibiot. (Tokyo), 57(7):421-428, 2004.
Huang et al., Cell, 27:245, 1981.
Hug et al., Mol. Cell. Biol., 8:3065, 1988.
Hui and Hashimoto, Infect. Immun., 66(11):5329-5336, 1998.
Hussussian et al., Nat. Genet., 8(1):15-21, 1994.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Inoue et al., Mol. Ther., 12:707-715, 2005.
Inouye and Inouye, Nucleic Acids Res., 13:3101-3109, 1985.
Irby and Yeatman, Oncogene, 19:5636-5642, 2000.
Irie and Morton, Proc. Natl. Acad. Sci. USA, 83(22):8694-8698, 1986.
Irie et al., Lancet., 1(8641):786-787, 1989.
Jagus et al., Int. J. Biochem. Cell. Biol., 31:123-138, 1999.
Jakobovits et al., Mol. Cell. Biol., 8:2555, 1988.
Jameel and Siddiqui, Mol. Cell. Biol., 6:710, 1986.
Jaynes et al., Mol Cell. Biol., 8:62, 1988.
Jemal et al, Cancer J. Clin., 52:23-47, 2002.
Jiang et al., Proc. Natl. Acad. Sci. USA, 93(17):9160-9165, 1996.
Jiang et al., Proc. Natl. Acad. Sci. USA, 93:9160-9165, 1996.
Johnson et al., Mol. Cell. Biol., 9:3393, 1989.
Ju et al., Gene Ther., 7(19):1672-1679, 2000.
Kabat et al., In: Sequences of Proteins of Immunological Interest, U.S. Department of Health and Human Services, Washington, D.C., 1987.
Kadesch and Berg, Mol. Cell. Biol., 6:2593, 1986.
Kamal et al., Nature., 425:407-410, 2003.
Kamb et al., Nat. Genet., 8(1):23-26, 1994.
Kaneda et al., Science, 243:375-378, 1989.
Kaplitt et al., Nat Genet., 8(2):148-154, 1994.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamori et al., Cancer Res 58(3): 409-12, 1998.
Kawamoto et al., Mol. Cell. Biol., 8:267, 1988.
Kerr et al., Br. J. Cancer, 26(4):239-257, 1972.
Kiledjian et al., Mol. Cell. Biol., 8:145, 1988.
Kismet et al., Cancer Detect. Prev., 10(6):589-601, 2004.
Klamut et al., Mol. Cell. Biol., 10:193, 1990.
Kline et al., In: Proceeding of the International Conference on Nutrition and Cancer, Prasad and Cole (Eds.), Amsterdam: IOS Press, 37-53, 1998.
Kline et al., J. Mammary Gland Biol. Neoplasia 8:91-102, 2003.
Kline et al., J. Nutr., 131: 161S-163S, 2001.
Knuefermann et al., Oncogene, 22:3205-3212, 2003.
Koch et al., Mol. Cell. Biol., 9:303, 1989.
Koch, et al., Rev. Physiol. Biochem. Pharmacol. 146:55-94, 2003.
Koehne and Dubois, Semin. Oncol., 31:12-21, 2004.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol Cell. Biol., 3:325, 1983.
Kriegler et al., Cell, 38:483, 1984.
Kriegler et al., Cell, 53:45, 1988.
Kuhl et al., Cell, 50:1057, 1987.
Kulp et al., Cancer Res., 64:1444-1451, 2004.
Kunz et al., Nucl. Acids Res., 17:1121, 1989.
Kyte and Doolittle, J. Mol. Biol., 157(1):105-132, 1982.
Ladenheim et al., Gastroenterology, 108:1083-1087, 1995.
LaFace et al., Virology, 162(2):483-486, 1988.
Lanza et al., Arch. Intern. Med., 155:1371-1377, 1995.
Larsen et al., Proc Natl. Acad. Sci. USA., 83:8283, 1986.
Laspia et al., Cell, 59:283, 1989.
Latimer et al, Mol. Cell. Biol., 10:760, 1990.
Laughlin et al., J. Virol., 60(2):515-524, 1986.
Lawson et al., Mol. Cancer Ther., 2:437-444, 2003.
Le Brazadec et al., J. Med. Chem., 47(15):3865-3873, 2004.
Le et al., J. Biol. Chem., 280(3):2092-2104, 2005.
Lebkowski et al., Mol. Cell. Biol., 8(10):3988-3996, 1988.
Lee et al., Nature, 294:228, 1981.
Lee et al., Nucleic Acids Res., 12:4191-206, 1984.
Leng et al., Hepatology, 38:756-768, 2003.
Levinson et al., Nature, 295:79, 1982.
Lin et al., Mol. Cell. Biol., 10:850, 1990.
Liu et al., Am. J. Clin. Oncol., 26:S103-109, 2003.
Liu et al., Cancer Res., 55(14):3117-3122, 1995.
Lupulescu, Cancer Detect. Prev., 20(6):634-637, 1996.
Luria et al, EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Samow, Nature, 353:90-94, 1991.
Majors and Vannus, Proc. Natl. Acad. Sci. USA, 80:5866, 1983.
Malafa and Neitzel, J. Surg. Res., 93:163-170, 2000.
Malafa et al., Surgery, 131:85-91, 2002.
Maloney and Workman, Expert Opin. Biol Ther., 2:3-24, 2002.
Mandler et al., Bioconjug. Chem., 13(4):786-791, 2002.
Mandler et al., Cancer Res., 64(4):1460-1467, 2004.
Mann et al., Cell, 33:153-159, 1983.
Marsters et al., Recent Prog. Horm. Res., 54:225-234, 1999.
Marte and Downward, Trends Biochem Sci., 22:355-358, 1997.
McCarty et al., J. Virol, 65(6):2936-2945, 1991.
McKenzie et al., Surgery, 136:437-442, 2004.
McLaughlin et al., J. Virol., 62(6):1963-1973, 1988.
McNeall et al., Gene, 76:81, 1989.
Mhashilkar et al., Mol. Med., 7(4):271-282, 2001.
Mhashilkar et al., Mol. Ther., 8:207-219, 2003.
Miksicek et al., Cell, 46:203, 1986.
Mitchell et al, Ann. NY Acad. Sci., 690:153-166, 1993.
Mitchell et al., J. Clin. Oncol., 8(5):856-869, 1990.
Mordacq and Linzer, Genes and Dev., 3:760, 1989.
Moreau et al., Nuc. Acids Res., 9:6047, 1981.
Mori et al., Cancer Res., 54(13):3396-3397, 1994.
Morton et al., Arch. Surg., 127:392-399, 1992.
Muesing et al., Cell, 48:691, 1987.
Muscat et al., Cancer, 74:1847-1854, 1994.
Muzyczka, Curr. Topics Microbiol. Immunol., 158:97-129, 1992.
Narisawa et al., Cancer Res., 41(5):1954-1957, 1981.
Neckers, Trends Mol. Med., 8(4 Suppl):S55-S56, 2002.
Neuzil et al., Br. J. Cancer, 84:87-89, 2000.
Neuzil et al., FASEB J., 15:403-415, 2001.
Ng et al., Nuc. Acids Res., 17:601, 1989.
Nicholson and Anderson, Cell Signal, 14(5):381-395, 2002.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nishikawa et al., Mol. Ther., 9:818-828, 2004.
Nishikawa et al., Oncogene, 23:7125-7131, 2004.
Nobri et al., Nature (London), 368:753-756, 1995.
Ohi et al., Gene, 89(2):279-282, 1990.
Okamoto et al., Proc. Natl. Acad. Sci. USA, 91(23):11045-11049, 1994.
Ondek et al., EMBO J., 6:1017, 1987.
Orlow et al., Cancer Res, 54(11):2848-2851, 1994.
Omitz et al., Mol. Cell. Biol., 7:3466, 1987.
Palmiter et al., Nature, 300:611, 1982.
Paskind et al., Virology, 67:242-248, 1975.
Pataer et al., Cancer Res., 62:2239-2243, 2002.
Patel et al., Chem. Biol., 11(12):1625-1633, 2004.
PCT Appln. WO 00/05356
PCT Appln. WO 00/26368
PCT Appln. WO 98/07408
PCT Appln. WO 98/28425
Pech et al., Mol. Cell. Biol., 9:396, 1989.
Pegram et al., J. Natl. Cancer Inst., 96:739-749, 2004.
Pelletier and Sonenberg, Nature, 334(6180):320-325, 1988.
Perez-Stable and Constantini, Mol. Cell. Biol., 10: 1116, 1990.
Pestka et al., Annu. Rev. Immunol., 22:929-979, 2004.
Peto et al., Lancet, 355:1822, 2000.
Piazza et al., Cancer Res., (55):311 3116, 1995.
Piazza et al., Cancer Res., (57):2452-2459, 1997b.
Piazza et al., Cancer Res., (57):2909-2915, 1997a.
Picard and Schaffner, Nature, 307:83, 1984.
Pietras et al., Oncogene, 17(17):2235-2249, 1998.
Pinkert et al., Genes and Dev., 1:268, 1987.
Pisani et al., Int. J. Cancer, 83:18-29; 870-873, 1999.
Pollard and Luckert, Cancer Res., 49:6471-6473, 1989.
Ponta et al., Proc. Natl. Acad. Sci. USA, 82:1020, 1985.
Porton et al., Mol. Cell Biol., 10:1076, 1990.
Prasad and Edwards-Prasad, J. Am. Coll. Nutr., 11:487-500, 1992.
Prasad and Edwards-Prasad., Cancer Res., 42:550-554, 1982.
Qin et al., Proc. Natl. Acad. Sci. USA, 95(24):14411-14416, 1998.
Queen and Baltimore, Cell, 35:741, 1983.
Quinn et al., Mol. Cell. Biol., 9:4713, 1989.
Rahmani et al., Cancer Res., 63:8420-8427, 2003.
Ramesh et al., Cancer Res., 63:5105-5113, 2003.
Ramesh et al., Mol. Ther., 3:337-350, 2001.
Rao et al., Cancer Res., 55(7):1464-1472, 1995.
Ravindranath and Morton, Intern. Rev. Immunol., 7: 303-329, 1991.
Reddy et al., Cancer Res., (50):2562-2568, 1990.
Reddy et al., Cancer Res., 47:5340-5346, 1987.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Resendez Jr. et al., Mol. Cell. Biol., 8:4579, 1988.
Ripe et al., Mol. Cell. Biol., 9:2224, 1989.
Ritland and Gendler, Carcinogenesis, 20(1): 51-58, 1999.
Rittling et al., Nuc. Acids Res., 17:1619, 1989.
Rosen et al., Cell, 41:813, 1988.
Rosenberg et al., Ann. Surg. 210(4):474-548, 1989.
Rosenberg et al., Nature Med., 4:321-327, 1998.
Ross et al., Oncologist, 8(4):307-25, 2003.
Saeki et al., Gene Ther., 7:2051-2057, 2000.
Saeki et al., Oncogene, 21:4558-4566, 2002.
Sakai et al., Genes and Dev., 2:1144, 1988.
Sambrook et al., In: Molecular cloning, Cold Spring Harbor Laboratory Press, Cold Spring Harbor, N.Y., 2001.
Samulski et al., J. Virol., 63:3822-3828, 1989.
Sarkar et al., Biotechniques Suppl., 30-39, 2002.
Satake et al., J. Virology, 62:970, 1988.
Sausville et al., Curr. Cancer Drug Targets, 3:377-383, 2003.
Schaefer et al., Cell Immunol., 214:110-122, 2001.
Schaffner et al., J. Mol. Biol., 201:81, 1988.
Schulte and Neckers, Cancer Chemother. Pharmacol., 42(4):273-279, 1998.
Schwartz and Shklar, J. Oral Maxillofac. Surg., 50:367-373, 1992.
Searle et al., Mol. Cell. Biol., 5:1480, 1985.
Serrano et al., Nature, 366:704-707, 1993.
Serrano et al., Science, 267(5195):249-252, 1995.
Sharp and Marciniak, Cell, 59:229, 1989.
Shaul and Ben-Levy, EMBO J., 6:1913, 1987.
Shelling and Smith, Gene Therapy, 1: 165-169, 1994.
Sherman et al., Mol. Cell. Biol., 9:50, 1989.
Simstein et al., Exp. Biol. Med., 228(9):995-1003, 2003.
Singh and Lippman, Oncology (Huntingt), 12(12): 1787-800, 1998.
Singh and Reddy, Annals. NY Acad. Sci., (768):205-209, 1995.
Singh et al., Carcinogenesis, (15):1317-1323, 1994.
Sleigh and Lockett, J. EMBO, 4:3831, 1985.
Smith et al., Cancer Chemother. Pharmacol., 54(6):475-486, 2004.
Smyth-Templeton et al., DNA Cell Biol., 21(12):857-867, 1997.
Solit et al., Cancer Res., 63:213921-213944, 2003.
Solodin et al., Biochemistry, 34(41):13537-13544, 1995.
Soo et al., J. Cell. Biochem., 74(1):1-10, 1999.
Spalholz et al., Cell, 42:183, 1985.
Spandau and Lee, J. Virology, 62:427, 1988.
Spandidos and Wilkie, EMBO J., 2:1193, 1983.
Steinbach et al., N. Engl. J. Med., 342:1946-1952, 2000.
Stephens and Hentschel, Biochem. J., 248:1, 1987.
Stuart et al., Nature, 317:828, 1985.
Su et al., Cancer Res., 58, 2339-2342, 1998.
Su et al., Proc. Natl. Acad. Sci. USA, 95:14400-14405, 1998.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tantivejkul et al., Breast Cancer Res. Treat., 79:301-312, 2003.
Tavernier et al, Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Taylor et al., Science, 285:107-110, 1999.
Temin, In: Gene Transfer, Kucherlapati (Ed.), NY, Plenum Press, 149-188, 1986.
Templeton et al., Nat. Biotechnol., 15:647-652, 1997.
Thierry et al., Proc. Natl. Acad. Sci. USA, 92(21):9742-9746, 1995.
Thiesen et al., J. Virology, 62:614, 1988.
Thompson et al., J. Natl. Cancer Inst., (87):125-1260, 1995.
Thun et al., N. Engl. J. Med., 325:1593-1596, 1991.
Tian et al., Bioorg. Med. Chem., 12(20):5317-5329, 2004.
Traber et al., Am. J. Clin. Nutr., 48, 1988.
Traber, Biofactors, 10(2-3):115-120, 1999.
Tratschin et al., Mol. Cell. Biol., 4:2072-2081, 1984.
Tratschin et al., Mol. Cell. Biol., 5:3258-3260, 1985.
Treisman, Cell, 42:889, 1985.
Tronche et al., Mol. Biol. Med., 7:173, 1990.
Trudel and Constantini, Genes and Dev., 6:954, 1987.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tyndell et al., Nuc. Acids. Res., 9:6231, 1981.
Vane, and Botting, Sem. in Arthritis and Rheumatism, (26):2-10, 1997.
Vannice and Levinson, J. Virology, 62:1305, 1988.
Vasseur et al., Proc Natl. Acad. Sci. USA, 77:1068, 1980.
Vorburger et al., Oncogene, 21:6278-6288, 2002.
Walsh et al., J. Clin. Invest, 94:1440-1448, 1994.
Walsh et al., J. Clin. Invest., 94(4):1440-1448, 1994.
Walter and Johnson, Annu. Rev. Cell Biol. 10:87-119, 1994.
Wang and Calame, Cell, 47:241, 1986.
Wang et al., J. Biol. Chem., 277(9):7341-7347, 2002.
Weber et al., Cell, 36:983, 1984.
Weber et al., Clin. Cancer Res., 8:863-869, 2002.
Wechter et al., Cancer Res., 57:4316-4324, 1997.
Wei et al., Gene Ther., 1(4):261-268, 1994.
Weinberger et al. Mol. Cell. Biol., 8:988, 1984.
Whitesell et al., Proc. Natl. Acad. Sci. USA, 91(18):8324-8328, 1994.
Williams, Sci STKE, 89:RE2, 2001.
Winde et al., Cancer Lett., 134(2): 201-7, 1998.
Winer et al., In: Cancer: Principles and Practice of Oncology, Devita et al., (Eds.), Lippincott-Raven, 6^{th} Ed., 1651-1716, PA, 2000.
Winoto and Baltimore, Cell, 59:649, 1989.
Wong et al., Gene, 10:87-94, 1980.
Wu et al., World J. Gastroenterol., 7:60-65, 2001.
Yang and Huang, Gene Therapy, 4 (9):950-960, 1997.
Yang et al., J. Virol., 68(8):4847-4856, 1994.
Yoder et al., Blood, 82(Suppl.):347A, 1994.
You et al., Cell Growth Differ., 12:471-480, 2001.
You et al., Mol. Carcinogenesis, 33:228-236, 2002.
Yu et al., Cancer Res., 61:6569-6576, 2001.
Yutzey et al. Mol. Cell. Biol., 9:1397, 1989.
Zhang et al., J. Biol. Chem., 275:24436-24443, 2000.
Zhou et al., Exp. Hematol., 21 (7):928-933, 1993.
Zhou et al., J. Exp. Med., 179(6):1867-1875, 1994.
Zhu et al., Science, 261(5118):209-211, 1993.

1. A method for treating cancer in a patient comprising providing MDA-7 and a COX-2 inhibitor to the patient.

2. The method of claim 1, wherein the MDA-7 is provided to the patient by administering to the patient a composition comprising a nucleic acid having a sequence encoding MDA-7 polypeptide, wherein the MDA-7 polypeptide is expressed in the patient.

3. (canceled)

4. The method of claim 2, wherein the nucleic acid is in a viral vector.

5.-6. (canceled)

7. The method of claim 4, wherein the vector is an adenovirus vector.

8.-11. (canceled)

12. The method of claim 1, wherein the MDA-7 is provided to the patient by administering to the patient a composition comprising purified MDA-7 protein.

13.-19. (canceled)

20. The method of claim 1, wherein the COX-2 inhibitor is celecoxib, rofecoxib, valdecoxib, lumiracoxib, or etoricoxib, or a combination thereof.

21.-32. (canceled)

33. A method for treating breast cancer in a patient comprising administering to the patient of i) an adenovirus vector comprising a nucleic acid sequence encoding MDA-7, wherein the nucleic acid sequence is under the control of a promoter capable of being expressed in the patient; and, ii) a COX-2 inhibitor.

34. The method of claim 33, wherein the patient is HER-2/neu negative.

35. The method of claim 33, further comprising resecting all or part of one or more tumors from the patient.

36.-43. (canceled)

44. The method of claim 33, wherein the COX-2 inhibitor is celecoxib, rofecoxib, valdecoxib, lumiracoxib, or etoricoxib, or a combination thereof.

45.-49. (canceled)

50. A method for radiosensitizing cancer cells comprising providing to the cells an amount of MDA-7 and a COX-2 inhibitor.

51. The method of claim 50, further comprising subjecting the cancer cells to radiation.

52.-59. (canceled)

60. A method for treating cancer in a patient comprising providing MDA-7 and a Hsp90 inhibitor to the patient.

61. The method of claim 60, wherein the MDA-7 is provided to the patient by administering to the patient a composition comprising a nucleic acid having a sequence encoding MDA-7 polypeptide, wherein the MDA-7 polypeptide is expressed in the patient.

62. (canceled)

63. The method of claim 61, wherein the nucleic acid is in a viral vector.

64.-65. (canceled)

66. The method of claim 63, wherein the vector is an adenovirus vector.

67.-70. (canceled)

71. The method of claim 60, wherein the MDA-7 is provided to the patient by administering to the patient a composition comprising purified MDA-7 protein.

72.-78. (canceled)

79. The method of claim, 60 wherein the Hsp90 inhibitor is geldanamycin, or a derivative or analog thereof, or a combination thereof.

80. The method of claim 79, wherein the Hsp90 inhibitor is a geldanamycin analog.

81.-90. (canceled)

91. A method for treating lung cancer in a patient comprising administering to the patient i) an adenovirus vector comprising a nucleic acid sequence encoding MDA-7, wherein the nucleic acid sequence is under the control of a promoter capable of being expressed in the patient; and, ii) a Hsp90 inhibitor.

92.-100. (canceled)

101. The method of claim 91, wherein the Hsp90 inhibitor is geldanamycin, or a derivative or analog thereof, or a combination thereof.

102.-113. (canceled)

114. A method for treating cancer in a patient comprising providing to the patient MDA-7 and an MDA-7 conjunctive agent.

115. The method of claim 114, wherein the conjunctive agent is a COX-2 inhibitor.

116.-117. (canceled)

118. A method for treating cancer in a patient comprising providing MDA-7 and a vitamin E compound to the patient.

119. The method of claim 118, wherein the MDA-7 is provided to the patient by administering to the patient a composition comprising a nucleic acid having a sequence encoding MDA-7 polypeptide, wherein the MDA-7 polypeptide is expressed in the patient.

120. (canceled)

121. The method of claim 119, wherein the nucleic acid is in a viral vector.

122.-123. (canceled)

124. The method of claim 121, wherein the vector is an adenovirus vector.

125.-128. (canceled)

129. The method of claim 118, wherein the MDA-7 is provided to the patient by administering to the patient a composition comprising purified MDA-7 protein.

130. (canceled)

131. The method of claim 118, wherein the patient is provided with between about 5 and about 500 mg of the vitamin E compound for each treatment.

132.-133. (canceled)

134. The method of claim 118, wherein the vitamin E compound is provided to the patient by administering to the patient a composition comprising a tocopherol or an esterified form thereof.

135.-137. (canceled)

138. The method of claim 118, wherein the patient is provided with a composition comprising the vitamin E compound, or an esterified form thereof, and either i) purified MDA-7 protein or ii) a nucleic acid having a sequence encoding MDA-7.

139.-150. (canceled)

151. A method for treating cancer in a patient comprising administering to the patient i) an adenovirus vector comprising a nucleic acid sequence encoding MDA-7, wherein the nucleic acid sequence is under the control of a promoter capable of being expressed in the patient; and, ii) alpha-tocopherol or an esterified form of alpha-tocopherol.

152.-180. (canceled)

181. A method for treating cancer in a patient comprising providing of MDA-7 and a tumor necrosis factor (TNF) to the patient.

182.-183. (canceled)

184. The method of claim 181, wherein the MDA-7 is provided to the patient by administering to the patient a composition comprising a nucleic acid having a sequence encoding MDA-7 polypeptide, wherein the MDA-7 polypeptide is expressed in the patient.

185. (canceled)

186. The method of claim 184, wherein the nucleic acid is in a viral vector.

187.-188. (canceled)

189. The method of claim 186, wherein the vector is an adenovirus vector.

190.-193. (canceled)

194. The method of claim 181, wherein the MDA-7 is provided to the patient by administering to the patient a composition comprising purified MDA-7 protein.

195. (canceled)

196. The method of claim 181, wherein the TNF is provided to the patient by administering to the patient a composition comprising a nucleic acid having a sequence encoding a TNF, wherein the TNF is expressed in the patient.

197.-205. (canceled)

206. The method of claim 196, wherein the TNF is provided to the patient by administering to the patient a composition comprising purified TNF.

207.-217. (canceled)

218. The method of claim 181, further comprising subjecting the patient to radiotherapy and/or chemotherapy.

219.-221. (canceled)

222. The method of claim 181, further comprising resecting all or part of a tumor from the patient.

223.-240. (canceled)

241. A method for treating cancer in a patient comprising providing to the patient MDA-7 and a vascular endothelial growth factor (VEGF) inhibitor.

242. The method of claim 241 wherein the inhibitor specifically binds VEGF or a VEGF receptor.

243. The method of claim 242, wherein the VEGF inhibitor is a DNA, an RNA, an oligonucleotide, a ribozyme, a protein, a polypeptide, a peptide, or a small molecule.

244. The method of claim 242, wherein the VEGF inhibitor is an antibody.

245. The method of claim 244, wherein the antibody is a monoclonal antibody.

246.-247. (canceled)

248. The method of claim 245, wherein the monoclonal antibody is bevacizumab.

249.-253. (canceled)

254. The method of claim 241, wherein the MDA-7 is provided to the patient by administering to the patient a composition comprising a nucleic acid having a sequence encoding MDA-7 polypeptide, wherein the MDA-7 polypeptide is expressed in the patient.

255. (canceled)

256. The method of claim 254, wherein the nucleic acid is in a viral vector.

257.-258. (canceled)

259. The method of claim 256, wherein the vector is an adenovirus vector.

260.-263. (canceled)

264. The method of claim 241, wherein the MDA-7 is provided to the patient by administering to the patient a composition comprising purified MDA-7 protein.

265.-287. (canceled)

288. The method of claim 241, wherein the patient is provided with at least the following:
a) purified MDA-7 protein or a nucleic acid having a sequence encoding MDA-7; and
b) a monoclonal antibody that specifically binds VEGF.

289. The method of claim 288, wherein the monoclonal antibody is bevacizumab.

290.-299. (canceled)

300. The method of claim 241, further comprising resecting all or part of a tumor from the patient.

301.-320. (canceled)

321. A pharmaceutical composition comprising:
a) purified and active MDA-7 protein or a nucleic acid having a sequence encoding MDA-7 polypeptide; and
b) a COX-2 inhibitor, an Hsp90 inhibitor, a vitamin E compound, purified and active TNF or a nucleic acid encoding TNF, or purified and active VEGF inhibitor or a nucleic acid encoding the VEGF inhibitor, or a prodrug thereof.

322. The pharmaceutical composition of claim 321, wherein the nucleic acid having a sequence encoding MDA-7 polypeptide is in an adenovirus vector.


SEQUENCE LISTING (28 sequences)

SEQ ID NO: 1 - DNA, 718 bp, Homo sapiens
acaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - DNA, 3634 bp, Homo sapiens
gaattccgggtgatttcactcccggctgtccaggcttgtcctgctaccccacccagcctttcctgaggcctcaagcctgccaccaagcccccagctccttctccccgcaggacccaaacacaggcctcaggactcaacacagcttttccctccaacccgttttctctccctcaacggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagaggtgagtgcctggccagccttcatccactctcccacccaaggggaaatgagagacgcaagagagggagagagatgggatgggtgaaagatgtgcgctgatagggagggatgagagagaaaaaaacatggagaaagacggggatgcagaaagagatgtggcaagagatggggaagagagagagagaaagatggagagacaggatgtctggcacatggaaggtgctcactaagtgtgtatggagtgaatgaatgaatgaatgaatgaacaagcagatatataaataagatatggagacagatgtggggtgtgagaagagagatgggggaagaaacaagtgatatgaataaagatggtgagacagaaagagcgggaaatatgacagctaaggagagagatgggggagataaggagagaagaagatagggtgtctggcacacagaagacactcagggaaagagctgttgaatgctggaaggtgaatacacagatgaatggagagagaaaaccagacacctcagggctaagagcgcaggccagacaggcagccagctgttcctcctttaagggtgactccctcgatgttaaccattctccttctccccaacagttccccagggacctctctctaatcagccctctggcccaggcagtcagtaagtgtctccaaacctctttcctaattctgggtttgggtttgggggtagggttagtaccggtatggaagcagtgggggaaatttaaagttttggtcttgggggaggatggatggaggtgaaagtaggggggtattttctaggaagtttaagggtctcagctttttcttttctctctcctcttcaggatcatcttctcgaaccccgagtgacaagcctgtagcccatgttgtaggtaagagctctgaggatgtgtcttggaacttggagggctaggatttggggattgaagcccggctgatggtaggcagaacttggagacaatgtgagaaggactcgctgagctcaagggaagggtggaggaacagcacaggccttagtgggatactcagaacgtcatggccaggtgggatgtgggatgacagacagagaggacaggaaccggatgtggggtgggcagagctcgagggccaggatgtggagagtgaaccgacatggccacactgactctcctctccctctctccctccctccagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaacatggtctccttcttggaattaattctgcatctgcctcttcttgtgggtgggaagaagctccctaagtcctctctccacaggctttaagatccctcggacccagtcccatccttagactcctagggccctggagaccctacataaacaaagcccaacagaatattccccatcccccaggaaacaagagcctgaacctaattacctctccctcagggcatgggaatttccaactctgggaattc

SEQ ID NO: 4 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 5 - DNA, 2140 bp, Homo sapiens
ccgacctagaacccgcccgctgcctgccacgctgccactgccgcttcctctataaagggacctgagcgtccgggcccaggggctccgcacagcaggtgaggctctcctgccccatctccttgggctgcccgtgcttcgtgctttggactaccgccccgcagtgtcctgccctctgcctgggcctcggtccctcctgcacctgctgcctggatccccggcctgcctgggcctgggccttggtgggtttggttttggtttccttctctgtctctgactctccatctgtcagtctcattgtctctgtcacacattctctgtttctgccatgattcctctctgttcccttcctgtctctctctgtctccctctgctcaccttggggtttctctgactgcatcttgtccccttctctgtcgatctctctctcgggggtcggggggtgctgtctcccagggcgggaggtctgtcttccgccgcgtgccccgccccgctcactgtctctctctctctctctctttctctgcaggttctccccatgacaccacctgaacgtctcttcctcccaagggtgtgtggcaccaccctacacctcctccttctggggctgctgctggttctgctgcctggggcccaggtgaggcagcaggagaatgggggctgctggggtggctcagccaaaccttgagccctagagcccccctcaactctgttctcccctaggggctccctggtgttggcctcacaccttcagctgcccagactgcccgtcagcaccccaagatgcatcttgcccacagcaccctcaaacctgctgctcacctcattggtaaacatccacctgacctcccagacatgtccccaccagctctcctcctacccctgcctcaggaacccaagcatccacccctctcccccaacttcccccacgctaaaaaaaacagagggagcccactcctatgcctccccctgccatcccccaggaactcagttgttcagtgcccacttcctcagggattgagacctctgatccagacccctgatctcccacccccatcccctatggctcttcctaggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccccctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggaccacacctccctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctgaaatgctgtctgcttgtgtgtgtgtg

SEQ ID NO: 6 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKAPSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 7 - DNA, 3410 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 8 - protein, 327 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 9 - DNA, 3542 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 10 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 11 - DNA, 3614 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 12 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 13 - DNA, 3665 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 14 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 15 - DNA, 1172 bp, Homo sapiens
gcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaaaaaaaaaaaaaaaaaaaaaaaaaaaaaaaaaaaaaaaaaa

SEQ ID NO: 16 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 17 - DNA, 2076 bp, Homo sapiens
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 18 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 19 - DNA, 2128 bp, Homo sapiens
caagacttctctgcattttctgccaaaatctgtgtcagatttaagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 20 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 21 - DNA, 1758 bp, Homo sapiens
ctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaaaaaaaaaaaaaaaaaaaaaaaaaaaaa

SEQ ID NO: 22 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 23 - protein, 11 aa, Rattus norvegicus
KTSQNIFENLA

SEQ ID NO: 24 - protein, 7 aa, Rattus norvegicus
NASPLQA

SEQ ID NO: 25 - protein, 8 aa, Rattus norvegicus
HQYYSGYT

SEQ ID NO: 26 - protein, 10 aa, Rattus norvegicus
GFTFSDYHMA

SEQ ID NO: 27 - protein, 17 aa, Rattus norvegicus
SITLDATYTYYRDSVRG

SEQ ID NO: 28 - protein, 10 aa, Rattus norvegicus
HRGFSVWLDY